SEQUENCE LISTING (25 sequences)

SEQ ID NO: 1 - DNA, 22 bp, Artificial sequence
  Chemically synthesized
cctggcattcctgatactcttc

SEQ ID NO: 2 - DNA, 22 bp, Artificial sequence
  Chemically synthesized
attctctgatttcagctctgcc

SEQ ID NO: 3 - DNA, 21 bp, Artificial sequence
  Chemically synthesized
tgctaggtaacaagcgaatgg

SEQ ID NO: 4 - DNA, 21 bp, Artificial sequence
  Chemically synthesized
caacaacaacaagggaaatgg

SEQ ID NO: 5 - protein, 249 aa, Mus musculus
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 6 - DNA, 842 bp, Mus musculus
cgcctcccaggcgctcggctgtgtcaagatgaagctgaacatctccttccccgccaccggctgtcagaagctcatcgaggtggatgacgagcgcaagctccgcaccttctatgagaagcgcatggccacggaagtagccgctgatgctcttggtgaagagtggaagggttatgtggtccggatcagcggtgggaatgacaagcaaggttttcccatgaagcaaggtgttctgacccatggcagagtgcgcctgctgttgagtaaggggcattcctgttacaggccaaggagaactggagagaggaagcgcaagtctgttcgtggatgcattgtggacgctaatctcagtgttctcaacttggtcattgtaaagaaaggagagaaggatattcctggactgacagacactactgtgcctcgtcggttgggacctaaaagggctagtagaatccgcaagctttttaatctctccaaagaagatgatgtccgccagtatgttgtcaggaagcccttaaacaaagaaggtaagaagcccaggaccaaagcacccaagattcagcgacttgttactcctcgtgtcctgcaacacaaacgccgacgtattgctctgaagaagcaacgcactaagaagaacaaggaggaggctgcagaatacgctaaacttttggccaagagaatgaaggaagccaaagaaaagcgccaggaacagattgccaagagacgtaggctgtcctcactgagagcttctacttctaagtctgagtccagtcaaaaatgagtctttaagagcaacaaataaataatgaccttgaatctttaaaaaaaaaaaaaaaaaaaaaaaa

SEQ ID NO: 7 - protein, 249 aa, Rattus norvegicus; identical to SEQ ID NO: 5

SEQ ID NO: 8 - DNA, 741 bp, Rattus novegicus
gtcggctgtgtcaagatgaagctgaatatctccttccctgccactggctgtcagaaactcatagaagtggatgacgaacgcaagcttcgtacgttctatgagaagcgcatggccacagaaaatgacaaacaaggttttcccatgaagcaaggcgttttgacccatggcagagtgcgcctgcttttgagtaaggggcattcttgttatagacctaggagaactggagagaggaagcgcaagtctgtccgaggatgcattgtggatgccaacctgagtgttctcaacttggttattgtaaaaaaaggagagaaggatattccaggactgacagataccactgtgcctcgtcggttgggacctaaaagagctagtagaatccgaaagctttttaatctctccaaagaagatgatgtccgccagtatgttgttagaaagcccttaaacaaagaaggtaagaagcccaggaccaaagcgcccaagattcagcgtcttgttactccccgtgtcctgcaacacaaacgccgacgtattgctctgaagaagcaacgcactaagaaaaacaaggaggaggctgcagaatatgctaaacttttggccaagagaatgaaggaagccaaagagaagcgccaggaacagattgccaagagacgtaggctgtcttcgctgagagcttctacttctaaatctgagtccagtcaaaaataagtctttaaagagtaacaaataaataatgagaccttg

SEQ ID NO: 9 - protein, 249 aa, Pan troglodytes; identical to SEQ ID NO: 5

SEQ ID NO: 10 - DNA, 895 bp, Pan troglodytes
tcgcgagaactgaaagcgcctatgtgacctgcgctaagcggaagttggcccttttttccgtggcgcctcggaggcgttcagctgcttcaagatgaagctgaacatctccttcccagccactggctgccaaaaactcattgaagtggacgatgaacgcaaacttcgtactttttatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccaaagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactcggatctctacaaaaaaaag

SEQ ID NO: 11 - protein, 249 aa, Homo sapiens [sequence omitted]

SEQ ID NO: 12 - DNA, 834 bp, Homo sapiens
ctcggaggcgttcagctgcttcaagatgaagctgaacatctccttcccagccactggctgccagaaactcattgaagtggacgatgaacgcaaacttcgtactttctatgagaagcgtatggccacagaagttgctgctgacgctctgggtgaagaatggaagggttatgtggtccgaatcagtggtgggaacgacaaacaaggtttccccatgaagcagggtgtcttgacccatggccgtgtccgcctgctactgagtaaggggcattcctgttacagaccaaggagaactggagaaagaaagagaaaatcagttcgtggttgcattgtggatgcaaatctgagcgttctcaacttggttattgtaaaaaaaggagagaaggatattcctggactgactgatactacagtgcctcgccgcctgggccccaaaagagctagcagaatccgcaaacttttcaatctctctaaagaagatgatgtccgccagtatgttgtaagaaagcccttaaataaagaaggtaagaaacctaggaccagagcacccaagattcagcgtcttgttactccacgtgtcctgcagcacaaacggcggcgtattgctctgaagaagcagcgtaccaagaaaaataaagaagaggctgcagaatatgctaaacttttggccaagagaatgaaggaggctaaggagaagcgccaggaacaaattgcgaagagacgcagactttcctctctgcgagcttctacttctaagtctgaatccagtcagaaataagattttttgagtaacaaataaataagatcagactctgaaaaaaaaaaaaaaaaaaaaaa

SEQ ID NO: 13 - protein, 249 aa, Bos taurus
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKDGKKPRTKAPKIQRLVTPRVLQHKRRRIALKKQRTKKNKEEAAEYAKLLAKRMKEAKEKRQEQIAKRRRLSSLRASTSKSESSQK

SEQ ID NO: 14 - DNA, 817 bp, Bos taurus
gcgcctcggaggctgtcggccgcttcagaatgaagctgaacatctctttcccggccactggctgccagaagctcattgaagtggacgatgaacgaaaacttcgtaccttctacgagaagcgtatggccacagaagttgctgctgacgctctgggtgaagaatggaagggttatgtggtccgaatcagtggcgggaacgataagcagggtttccccatgaagcagggtgtcttgacccatggcagagttcgcctgctactgagtaaggggcattcctgttacagaccaaggaggactggagagagaaagcgcaaatctgtacggggttgcattgtggatgccaatctgagtgttctcaatttggtcatcgtgaaaaaaggggaaaaggatattcctggactcactgatactacagtgcctcgtcgcctgggtcccaaaagagccagcagaatccgcaaacttttcaatctctctaaagaagatgatgtccgccaatatgttgtgcgaaagcccctaaacaaagacggtaagaaacctaggactaaagcacccaagattcagcgtctcgtgactccacgagttctgcagcacaaacgccggcgtattgctctgaagaaacagcgtactaagaaaaataaagaagaggctgcagaatatgctaaacttttggccaagagaatgaaggaggccaaagaaaaacggcaggaacagattgccaagagacggaggctgtcctctctgagagcttctacttctaagtctgagtccagtcaaaaatgagatgttctaagagtaacaaataaataagatcagacatc

SEQ ID NO: 15 - protein, 249 aa, Gallus gallus
MKLNISFPATGCQKLIEVDDERNVRTFYEKRMATEVAADSLGEEWKGYVVRISGGNDKQGFPMKQGVLTHGRVRLLLSKGHSCYRPRRTGERKRKSVRGCIVDANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTQKNKEEAADYAKLLAKRMKEAKEKRQEQIAKRRRLSSLRASTSKSESSQK

SEQ ID NO: 16 - DNA, 894 bp, Gallus gallus
ccggcgcagttcggcgaggatgaagctcaacatctctttcccagccactggctgccagaagcttattgaagtggatgatgagcgcaacgtgagaacattctatgagaagcgaatggccacggaggttgcggctgattctcttggcgaggagtggaagggctatgttgtccggatcagtggtggcaatgataaacaaggcttccccatgaagcagggtgtccttactcatggacgtgtccgccttctgctcagcaaaggccactcctgctaccgccccaggagaactggagagagaaaacgcaagtctgttcggggttgcattgttgacgccaacttgagtgttctgaacttggtcattgtgaaaaagggtgagaaggatattcctgggctgacagacacaactgtgcctcgtcgtcttggtcccaagagagctagcaggatccgcaagctgttcaatctctctaaggaagatgatgttcgccagtatgttgtgaggaaacctctgaataaagagggcaagaaacccaggaccaaggctcctaagatccagcgactagtgactcctcgtgttctgcaacataagcgcagacgtattgccctgaagaagcagcgcactcagaagaacaaggaggaggcagcagattacgcgaagctcttggcaaagagaatgaaggaggccaaggaaaaacgccaggagcagattgcgaagagacgcaggctttcttcattgagagcttctacatctaaatctgagtcaagtcagaagtaaagatgtacatgatactgaaaataaaacccttttgtggttaaattttactgtgagacttccagtgaatatatttcctggctatgtcttaaaataaatggtagtccagactaaaaaaaaaaaaaaaa

SEQ ID NO: 17 - protein, 249 aa, Oryctolagus cuniculus
MKLNISFPATGCQKLIEVDDERKLRTFYEKRMATEVAADALGEEWKGYVVRISGGNDKQGFPMKQGVLTHGRVRLLLSKGHSCYRPRRTGERKRKSVRGCIVNANLSVLNLVIVKKGEKDIPGLTDTTVPRRLGPKRASRIRKLFNLSKEDDVRQYVVRKPLNKEGKKPRTKAPKIQRLVTPRVLQHKRRRIALKKQRTKKNKEEAAEYAKFLAKRMKEAKEKRQEQIAKRCRLSSLRASTSKSESSQK

SEQ ID NO: 18 - DNA, 840 bp, Oryctolagus cuniculus
gcgcctccgagccggtcagctgcttcaaaatgaagctgaatatctccttcccagccactggctgccagaaactcatcgaagtggacgatgaacgtaaacttcgtactttctatgagaagcgtatggccacagaagttgctgccgatgctctgggtgaagaatggaagggttatgtggtccggatcagtggtgggaatgataaacaaggttttcccatgaagcaaggtgtcttgacccatgggcgggtccgcctgctgctgagtaaggggcattcctgttacagaccaaggagaactggagaaagaaagcgcaaatcagttcggggctgcattgtcaatgccaatttgagtgttctcaacttggttattgtaaaaaaaggagagaaagatattcctggattgactgataccacggtgcctcgtcgcctgggtcctaaaagagccagcagaattcgtaaacttttcaatctttctaaagaagatgatgtacgccagtatgttgtaagaaagcccttaaacaaagaaggtaagaaacctaggaccaaagcacccaagattcagcgtctggttactccacgtgtcctgcaacacaaacgccggcgaattgctctgaagaaacagcgtactaagaagaacaaggaggaggctgcagaatatgctaaattcttggccaagagaatgaaggaggccaaagaaaaacgccaggaacaaattgccaagagatgtaggctgtcttctctgagagcgtctacttctaaatctgagtccagtcaaaaataaggtttaatgacaacaaataaataagattgtgtttcagatctcctttaaaaaaaataataat

SEQ ID NO: 19 - protein, 249 aa, Xenopus tropicalis
MKLNISFPATGCQKLIEVEDERKLRTFYEKRMATEVAADPLGDEWKGYVVRISGGNDKQGFPMKQGVLTHGRVRLLLSKGHSCYRPRRTGERKRKSVRGCIVDANLSVLNLVIVRKGEKDIPGLTDNTVPRRLGPKRASRIRKLFNLSKEDDVRQYVVRKPLAKEGKKPRTKAPKIQRLVTPRVLQHKRRRIALKKQRTQKNKEEASEYAKLLAKRTKEAKEKRQEQIAKRRRLSSLRASTSKSESSQK

SEQ ID NO: 20 - DNA, 846 bp, Xenopus tropicalis
gggggatctaagacagactggttgttggccatgaagcttaacatctccttcccagccactggctgccaaaagctcatcgaagtggaggatgagcgcaagctgcgtaccttctatgagaagcgcatggctacagaggttgctgcagatcccttgggtgatgagtggaagggatatgtcgttcgcatcagcggtggaaatgataagcaaggctttcccatgaaacagggagtgctaactcatggccgtgttcgtcttctgttgagcaagggtcattcctgttatcgccccaggaggactggtgaacgcaagcgcaagtctgttcgtgggtgtattgtggatgctaacctgagtgtcctgaacttggttattgttaggaaaggcgagaaggatattcctggacttacagacaacactgttcctcgtcgcctgggtcccaaaagagccagcagaatccgcaaactgttcaacttgtcaaaagaagatgatgtgcgtcaatatgtagtgaggaagcctctggctaaggaggggaagaagcccaggaccaaggcccctaaaatccagcgtctagtgaccccgagagttctgcagcacaagcgcagacgtattgctttgaagaagcagcgcactcagaagaataaggaagaggcatcagagtatgctaaacttctggctaagagaacaaaggaagccaaggaaaaacgccaggagcaaattgccaagaggcgcagactgtcttctttgagagcctccacatccaaatctgaatcgagtcagaaataaaactccatcatgtaaaaataaatacattttgttgtaaacttaaaaaaaaaaaaaaaaaaaaaaaaa

SEQ ID NO: 21 - protein, 249 aa, Xenopus laevis
MKLNISFPATGCQKLIEVEDERKLRTFYEKRMATEVAADPLGDEWKGYVVRISGGNDKQGFPMKQGVLTHGRVRLLLSKGHSCYRPRRTGERKRKSVRGCIVDANLSVLNLVIVRKGEKDIPGLTDNTVPRRLGPKRASRIRKLFNLSKEDDVRQYVVRKPLAKEGKKPRTKAPKIQRLVTPRVLQHKRRRIALKKQRTQKNKEEASEYAKLLAKRSKEAKEKRQEQIAKRRRLSSLRASTSKSESSQK

SEQ ID NO: 22 - DNA, 861 bp, Xenopus laevis
gctctttccggcgggggatctaagctagtctggttgttggccatgaagcttaatatctcgttcccagccactggctgccaaaagctcattgaagtggaggatgagcgcaagctgcgtactttctatgagaagcgcatggccacagaggtcgccgcagatcccttgggtgatgagtggaagggatatgttgttcgcatcagcggtggaaacgataagcaaggcttccccatgaaacagggagtcctaactcatggtcgtgttcgtcttctattaagcaagggtcattcctgctatcgccccaggagaactggtgaacgcaagcgcaaatctgtacgtggatgtattgtggatgctaacctcagtgtcctgaacttggttattgttaggaaaggtgaaaaggatattcctggcctgacagacaacactgttcctcgtcgcctgggtcccaaaagagccagcagaatccgcaaactattcaacttgtccaaagaagatgatgtgcgtcagtatgtagtgagaaagcctctggctaaggaagggaaaaagcccaggaccaaggcccctaaaatccagcgtctagtgacccccagagttctacagcataagcgcagacgtattgctttgaagaagcagcgtactcaaaagaataaggaagaggcttcagaatatgccaaacttctggctaagagatcaaaggaagccaaggaaaaacgccaggagcagatcgcaaagaggcgtagactgtcttctttgagagcctccacatccaaatctgaatccagtcagaaataaagcttcatcatgtaaaaataaatacattttgttgtaaacaaaaaaaaaaaaaaaaaaaaaaaaaaaaaa

SEQ ID NO: 23 - protein, 249 aa, Danio rerio
MKLNISFPATGCQKLIEVDDERKLRIFYEKRMATEVAADSLGDEWKGYVVRISGGNDKQGFPMKQGVLTHGRVRLLLSKGHSCYRPRRTGERKRKSVRGCIVDANLSVLNLVIVRKGEKDIPGLTDSTVPRRLGPKRASRIRKLFNLSKEDDVRQYVVRRPLTKEGKKPRTKAPKIQRLVTPRVLQHKRRRIALKRQRTLKNKEAAAEYTKLLAKRMKEAKEKRQEQIAKRRRLSSLRASTSKSESSQK

SEQ ID NO: 24 - DNA, 842 bp, Danio rerio
ctccaagcgagaaagtcctccatcatgaagctcaatatctcgttccccgccaccggctgccaaaagctgatagaagttgacgatgaacgcaagctgagaatcttctacgagaagcgcatggccacagaggtggctgcagactctctgggtgacgagtggaagggctacgttgtgcgcatcagcggaggcaatgacaaacagggcttccccatgaagcagggtgtgctgacccatggacgtgtgcgtctcctcctcagcaagggtcactcttgttaccgtcctcgccgtactggtgagcgcaaacgcaagtctgtccgcggctgcatcgtcgacgccaacctgagtgttctcaacttggtcattgtcaggaagggtgagaaggatattcctgggctgactgatagcactgtccctcgccgtctgggacccaagagggctagcaggatccgcaagctcttcaacctgtccaaagaggacgatgtcaggcagtatgtggtccggagacctctcactaaagaaggcaagaagcccaggactaaagcccctaagattcagcgtctggttacaccccgtgtgctgcagcacaagcgcagacgcatcgctctcaagaggcagcgcacactgaagaacaaggaggcagcagcagaatacaccaaactgctggccaagaggatgaaggaggccaaggagaaacgtcaagaacagattgctaagagacgccgtctttcctctctgagagcctccacatccaagtcagagtcaagccagaagtgagacatgtacctcacaaataaaacatgattttttgaaacattctaaaaaaaaaaaaaaaaaaaaaaaaa

SEQ ID NO: 25 - protein, 23 aa, Artificial Sequence
  Chemically synthesized
QIAKRRRLXXLRAXTSKSESSQK

(19) United States
(12) Patent Application Publication
(10) Pub. No.: US 2015/0141274 A1
(43) Pub. Date: May 21, 2015

(54) Methods and Compositions for Activity Dependent Transcriptome Profiling

(71) Applicant: The Rockefeller University, New York, NY (US)

(72) Inventors: Jeffrey M. Friedman, New York, NY (US); Zachary A. Knight, New York, NY (US); Keith Tan, New York, NY (US); Kivan Birsoy, New York, NY (US)

(21) Appl. No.: 14/399,057

(22) PCT Filed: May 9, 2013
(86) PCT No.: PCT/US13/40305

§ 371 Date: Nov. 5, 2014

Related U.S. Application Data
(60) Provisional application No. 61/645,035, filed on May 9, 2012.

(51) Int. Cl. C12N 15/10; C12Q 1/68; C07K 14/00

(52) U.S. Cl. CPC C12N 15/1041; C12N 15/1006; C07K 14/001; C12Q 1/6809
USPC 506/9; 536/25.41; 435/6.19; 435/6.13; 435/287.2; 530/387.9; 435/331; 530/326; 435/6.11

(57) ABSTRACT

Disclosed herein are methods, compositions, and kits for isolating actively translated mRNA from heterogeneous cell populations. Also disclosed herein are methods, compositions, and kits for identifying cell types that respond to stimuli in heterogeneous cell populations.

### CROSS-REFERENCE TO RELATED APPLICATIONS

This application claims priority to U.S. Provisional Patent Application No. 61/645,035, filed May 9, 2012, the contents of which are incorporated by reference herein in its entirety.

### BACKGROUND OF THE INVENTION

Cellular heterogeneity poses a challenge for those seeking to characterize the modulation of gene expression in complex tissues in response to various stimuli because only a subpopulation of the cells in such tissue may be activated or effected by such stimuli. The enormous heterogeneity of a tissue such as the nervous system (thousands of neuronal cell types, with non-neuronal cells outnumbering neuronal cells by an order of magnitude) can be a barrier to the identification and analysis of gene transcripts in a subpopulation of activated cell types. Cellular subtypes in such tissues can be highly heterogeneous and often intermixed. Gene expression studies on isolated cells have been limited by stresses introduced during cellular isolation procedures, the adaptations which occur upon the loss of tissue-intrinsic signals that control cellular physiology in vivo, and the technical challenges associated with reproducible mRNA purification from fixed tissue. There is a need in the art for methods of isolating and characterizing mRNAs whose translation is modulated by one or more stimuli without the need for cell isolation.

### SUMMARY OF THE INVENTION

Disclosed herein are methods of isolating actively translated mRNA from a first subpopulation of cells, the method comprising: (a) contacting a lysate or fraction of a heterogeneous population of cells with a reagent, the heterogeneous population of cells comprising the first subpopulation of cells and a second subpopulation of cells; (b) allowing the reagent to selectively bind to a protein comprising one or more posttranslational modifications, the protein being in a ribosome bound to the actively translated mRNA, (i) wherein the first and the second subpopulation of cells comprise more than one of the protein, (ii) wherein a greater percentage of the protein comprises at least one of the one or more posttranslational modifications in the first subpopulation of cells than in the second subpopulation of cells; and (c) isolating the actively translated mRNA from the lysate or fraction of the heterogeneous population of cells, thereby isolating actively translated mRNA from the first subpopulation of cells. In some embodiments, the isolating step comprises isolating the ribosome bound to the reagent and the actively translated mRNA. Some embodiments further comprise identifying the actively translated mRNA. Some embodiments further comprise determining an amount of the actively translated mRNA. In some embodiments, the amount of the actively translated mRNA is normalized based on the amount of the mRNA in the lysate or fraction prior to contacting the lysate or fraction with the reagent.

In some embodiments, the reagent binds to the protein at one or more sites of the one or more posttranslational modifications. In some embodiments, the one or more posttranslational modifications comprise myristoylation, palmitoylation, isoprenylation, glypiation, acylation, alkylation, amidation, butyrylation, gamma-carboxylation, glycosylation, malonylation, hydroxylation, iodination, oxidation, phosphorylation, adenylylation, proprionylation, pyroglutamate formation, nitrosylation, succinylation, sulfation, glycation, SUMOylation, ubiquitination, Neddylation, or a combination thereof. In some embodiments, at least one of the one or more posttranslational modifications is phosphorylation.

In some embodiments, the reagent comprises an antibody or fragment thereof, aptamer, or other ligand. In some embodiments, the reagent comprises a polyclonal antibody or fragment thereof. In some embodiments, the reagent comprises a monoclonal antibody or fragment thereof. In some embodiments, the reagent is a phospho-S6 240/244 antibody or fragment thereof. In some embodiments, the reagent is a phospho-S6 235/236 antibody or fragment thereof. In some embodiments, the reagent is a phospho-S6 244 antibody or fragment thereof.

In some embodiments, the reagent can specifically bind to the protein at two or more sites. In some embodiments, the two or more sites can comprise at least one of the one or more posttranslational modifications.

Some embodiment further comprise a peptide that decreases a binding affinity of the reagent for the protein at one or more of the two or more sites. Some embodiments comprise a peptide that increases the specificity of the reagent for at least one of the two or more sites. In some embodiments, the peptide comprises at least one of the one or more posttranslational modifications. In some embodiments, the peptide has at least about 60%, 70%, 80%, 90%, 95%, 99%, or 100% identity to SEQ ID NO:25.

In some embodiments, the protein is a ribosomal protein. In some embodiments, the protein is a large ribosomal subunit protein. In some embodiments, the protein is a small ribosomal subunit protein. In some embodiments, the protein is ribosomal protein S6. In some embodiments, the protein is ribosomal protein S6 and the one or more posttranslational modifications comprise phosphorylation at serine 235, serine 236, serine 240, serine 244, serine 247, or a combination thereof. In some embodiments, the protein is ribosomal protein S6 and at least one of the one or more posttranslational modifications is phosphorylation at serine 244. In some embodiments, the ribosomal protein S6 is a mouse protein. In some embodiments, at least one of the one or more posttranslational modifications occurs in response to a stimulus.

In some embodiments, the stimulus is an environmental stimulus, a dietary or metabolic stimulus, a drug or active agent, or a toxin. In some embodiments, the stimulus is an atypical antipsychotic. In some embodiments, the stimulus is amisulpride, aripiprazole, asenapine, blonanserin, clotiapine, clozapine, iloperidone, lurasidone, mosapramine, olanzepine, paliperidone, perospirone, quetiapine, remoxipride, risperidone, sertindole, sulpiride, ziprasidone, zotepine, bifeprunox, pimavanserin, vabicaserin, or a combination thereof.

In some embodiments, the heterogeneous population of cells comprises prokaryotic cells, eukaryotic cells, or a combination thereof. In some embodiments, the heterogeneous population of cells comprises mammalian cells. In some embodiments, the heterogeneous population of cells comprises mouse cells.

In some embodiments, the lysate or fraction is derived from all or a portion of a cell culture. In some embodiments, the lysate or fraction is derived from a tissue sample. In some embodiments, the lysate or fraction is derived from all or a portion of an organ. In some embodiments, the lysate or fraction is derived from all or a portion of a brain, stomach, intestine, lung, or a combination thereof.

Also disclosed herein are methods for identifying mRNA whose translation is modulated in response to a stimulus, the method comprising: (a) contacting a lysate or fraction of a heterogeneous population of cells with a reagent, (i) wherein the stimulus has been applied to a source of the heterogeneous population of cells, (ii) wherein the heterogeneous population of cells comprises a protein comprising one or more posttranslational modifications, and (iii) wherein at least one of the one or more posttranslational modifications occurs in response to the stimulus; (b) allowing the reagent to selectively bind to the protein comprising the one or more posttranslational modifications, the protein being in a ribosome bound to the mRNA; (c) isolating the ribosome bound to the reagent and the mRNA; (d) determining an identity and an amount of the mRNA in the isolated ribosome; (e) determining an identity and an amount of the mRNA in a control sample; and (f) comparing, for mRNA of common identity, the amount of the mRNA in the isolated ribosome to the amount of the mRNA in the control sample, thereby identifying mRNA whose translation is modulated in response to the stimulus.

Also disclosed herein are methods for identifying cell types that are activated in response to a stimulus, the methods comprising: (a) contacting a lysate or fraction of a heterogeneous population of cells with a reagent, (i) wherein the stimulus has been applied to a source of the heterogeneous population of cells, (ii) wherein the heterogeneous population of cells comprises a protein comprising one or more posttranslational modifications, and (iii) wherein at least one of the one or more posttranslational modifications occurs in response to the stimulus; (b) allowing the reagent to selectively bind to the protein comprising the one or more posttranslational modifications, the protein being in a ribosome bound to the mRNA; (c) isolating the ribosome bound to the reagent and the mRNA; (d) determining an identity and an amount of the mRNA in the isolated ribosome; (e) determining an identity and an amount of the mRNA in a control sample; (f) comparing, for mRNA of common identity, the amount of the mRNA in the isolated ribosome to the amount of the mRNA in the control sample to identify a profile of two or more mRNA whose translation is modulated in response to the stimulus; and (g) correlating the profile of two or more mRNA to genetic markers associated with one or more cell types in the heterogeneous population of cells, thereby identifying cell types that are activated in response to the stimulus. In some embodiments, prior to the comparing step, the amount of the mRNA in the isolated ribosome is normalized based on the amount of the mRNA in the lysate or fraction prior to contacting the lysate or fraction with the reagent. In some embodiments, the mRNA in the control sample is isolated using the reagent prior to the determining step. In some embodiments, prior to the comparing step, the amount of the mRNA in the control sample is normalized based on the amount of the mRNA in the control sample prior to isolation of the mRNA with the reagent. In some embodiments, wherein the mRNA in the control sample is isolated using a second reagent prior to the determining step. In some embodiments, the second reagent selectively binds to a second protein, the second protein being in a ribosome bound to the mRNA in the control sample. In some embodiments, the second protein is ribosomal protein L7 or ribosomal protein L26. In some embodiments, prior to the comparing step, the amount of the mRNA in the control sample is normalized based on the amount of the mRNA in the control sample prior to isolating the mRNA with the second reagent. In some embodiments, the control sample is a lysate or fraction of a corresponding heterogeneous population of cells from a source that has not been exposed to the stimulus. In some embodiments, the control sample is a lysate or fraction of a corresponding heterogeneous population of cells from a source that has been exposed to a different stimulus. In some embodiments, the source is an organism or cell culture. In some embodiments, the source is a mammal. In some embodiments, the source is a mouse. In some embodiments, the reagent binds to the protein at one or more sites of the one or more posttranslational modifications. In some embodiments, the one or more posttranslational modifications comprise myristoylation, palmitoylation, isoprenylation, glypiation, acylation, alkylation, amidation, butyrylation, gamma-carboxylation, glycosylation, malonylation, hydroxylation, iodination, oxidation, phosphorylation, adenylylation, proprionylation, pyroglutamate formation, nitrosylation, succinylation, sulfation, glycation, SUMOylation, ubiquitination, Neddylation, or a combination thereof. In some embodiments, at least one of the one or more posttranslational modifications is phosphorylation. In some embodiments, the reagent comprises an antibody or fragment thereof, aptamer, or other ligand. In some embodiments, the reagent comprises a polyclonal antibody or fragment thereof. In some embodiments, the reagent comprises a monoclonal antibody or fragment thereof. In some embodiments, the reagent is a phospho-S6 240/244 antibody or fragment thereof. In some embodiments, the reagent is a phospho-S6 235/236 antibody or fragment thereof. In some embodiments, the reagent is a phospho-S6 244 antibody or fragment thereof. In some embodiments, the reagent can specifically bind to the protein at two or more sites. In some embodiments, the two or more sites can comprise at least one of the one or more posttranslational modifications. Some embodiments, further comprise a peptide that decreases a binding affinity of the reagent for the protein at one or more of the two or more sites. In some embodiments, the peptide comprises at least one of the one or more posttranslational modifications. In some embodiments, the peptide has at least about 60%, 70%, 80%, 90%, 95%, 99%, or 100% identity to SEQ ID NO:25. Some embodiments further comprise a peptide that increases the specificity of the reagent for at least one of the two or more sites. In some embodiments, the peptide comprises at least one of the one or more posttranslational modifications. In some embodiments, the peptide has at least about 60%, 70%, 80%, 90%, 95%, 99%, or 100% identity to SEQ ID NO:25. In some embodiments, the protein is a ribosomal protein. In some embodiments, the protein is a large ribosomal subunit protein. In some embodiments, the protein is a small ribosomal subunit protein. In some embodiments, the protein is ribosomal protein S6. In some embodiments, the protein is ribosomal protein S6 and the one or more posttranslational modifications comprise phosphorylation on serine 235, serine 236, serine 240, serine 244, serine 247, or a combination thereof. In some embodiments, the protein is ribosomal protein S6 and at least one of the one or more posttranslational modifications is phosphorylation on serine 244. In some embodiments, the ribosomal protein S6 is a mouse protein. In some embodiments, the stimulus is an environmental stimulus, a dietary or metabolic stimulus, a drug or active agent, or a toxin. In some embodiments, the stimulus is an atypical antipsychotic. In some embodiments, the stimulus is amisulpride, aripiprazole, asenapine, blonanserin, clotiapine, clozapine, iloperidone, lurasidone, mosapramine, olanzepine, paliperidone, perospirone, quetiapine, remoxipride, risperidone, sertindole, sulpiride, ziprasidone, zotepine, bifeprunox, pimavanserin, vabicaserin, or a combination thereof. In some embodiments, the heterogeneous population of cells comprises prokaryotic cells, eukaryotic cells, or a combination thereof. In some embodiments, the heterogeneous population of cells comprises mammalian cells. In some embodiments, the heterogeneous population of cells comprises mouse cells. In some embodiments, the lysate or fraction is derived from all or a portion of a cell culture. In some embodiments, the lysate or fraction is derived from a tissue sample. In some embodiments, the lysate or fraction is derived from all or a portion of an organ. In some embodiments, the lysate or fraction is derived from all or a portion of a brain, stomach, intestine, lung, or a combination thereof.

Also disclosed herein are methods of isolating actively translated mRNA from activated cells, the methods comprising: (a) contacting a lysate or fraction of a heterogeneous population of cells with a reagent, the heterogeneous population of cells comprising activated cells and unactivated cells; (b) allowing the reagent to selectively bind to phosphorylated ribosomal protein S6, the phosphorylated ribosomal protein S6 being in a ribosome bound to the actively translated mRNA, (i) wherein the activated cells and unactivated cells comprise more than one of the ribosomal protein S6, and wherein a greater percentage of the ribosomal protein S6 is phosphorylated in the activated cells than in the unactivated cells; and (c) isolating the actively translated mRNA from the lysate or fraction of the heterogeneous population of cells, thereby isolating actively translated mRNA from activated cells. In some embodiments, the isolating step comprises isolating the ribosome bound to the reagent and the actively translated mRNA. Some embodiments further comprise identifying the actively translated mRNA. Some embodiments further comprise determining an amount of the actively translated mRNA. In some embodiments, the amount of the actively translated mRNA is normalized based on the amount of the mRNA in the lysate or fraction prior to contacting the lysate or fraction with the reagent. In some embodiments, the reagent binds to the phosphorylated ribosomal protein S6 at a site that is phosphorylated. In some embodiments, the reagent comprises an antibody or fragment thereof, aptamer, or other ligand. In some embodiments, the reagent comprises a polyclonal antibody or fragment thereof. In some embodiments, the reagent comprises a monoclonal antibody or fragment thereof. In some embodiments, the reagent is a phospho-S6 240/244 antibody or fragment thereof. In some embodiments, the reagent is a phospho-S6 235/236 antibody or fragment thereof. In some embodiments, the reagent is a phospho-S6 244 antibody or fragment thereof. In some embodiments, the reagent can specifically bind to the phosphorylated ribosomal protein S6 at two or more sites. In some embodiments, the two or more sites can be phosphorylated. Some embodiments further comprise a peptide that decreases a binding affinity of the reagent for the phosphorylated ribosomal protein S6 at one or more of the two or more sites. Some embodiments further comprise a peptide that increases the specificity of the reagent for at least one of the two or more sites. In some embodiments, the peptide is phosphorylated. In some embodiments, the peptide has at least about 60%, 70%, 80%, 90%, 95%, 99%, or 100% identity to SEQ ID NO:25. In some embodiments, the phosphorylated ribosomal protein S6 is phosphorylated at serine 235, serine 236, serine 240, serine 244, serine 247, or a combination thereof. In some embodiments, the phosphorylated ribosomal protein S6 is phosphorylated at serine 244. In some embodiments, the ribosomal protein S6 is a mouse protein. In some embodiments, the phosphorylated ribosomal protein S6 is phosphorylated in response to a stimulus. In some embodiments, the stimulus is an environmental stimulus, a dietary or metabolic stimulus, a drug or active agent, or a toxin. In some embodiments, the stimulus is an atypical antipsychotic. In some embodiments, the stimulus is amisulpride, aripiprazole, asenapine, blonanserin, clotiapine, clozapine, iloperidone, lurasidone, mosapramine, olanzepine, paliperidone, perospirone, quetiapine, remoxipride, risperidone, sertindole, sulpiride, ziprasidone, zotepine, bifeprunox, pimavanserin, vabicaserin, or a combination thereof. In some embodiments, the heterogeneous population of cells comprises prokaryotic cells, eukaryotic cells, or a combination thereof. In some embodiments, the heterogeneous population of cells comprises mammalian cells. In some embodiments, the heterogeneous population of cells comprises mouse cells. In some embodiments, the lysate or fraction is derived from all or a portion of a cell culture. In some embodiments, the lysate or fraction is derived from a tissue sample. In some embodiments, the lysate or fraction is derived from all or a portion of an organ. In some embodiments, the lysate or fraction is derived from all or a portion of a brain, stomach, intestine, lung, or a combination thereof.

Also disclosed herein are systems for isolating actively translated mRNA from a first subpopulation of cells, the systems comprising: (a) a lysate or fraction of a heterogeneous population of cells wherein the heterogeneous population of cells comprises a first subpopulation of cells and a second subpopulation of cells; (b) a reagent that selectively binds to a protein comprising one or more posttranslational modifications, the protein being in a ribosome bound to the actively translated mRNA, (i) wherein the first and the second subpopulation of cells comprise more than one of the protein, and (ii) wherein a greater percentage of the protein comprises at least one of the one or more posttranslational modifications in the first subpopulation of cells than in the second subpopulation of cells; and (c) a container configured to house the lysate or fraction and the reagent. In some embodiments, the reagent binds to the protein at one or more sites of the one or more posttranslational modifications. In some embodiments, the one or more posttranslational modifications comprise myristoylation, palmitoylation, isoprenylation, glypiation, acylation, alkylation, amidation, butyrylation, gamma-carboxylation, glycosylation, malonylation, hydroxylation, iodination, oxidation, phosphorylation, adenylylation, proprionylation, pyroglutamate formation, nitrosylation, succinylation, sulfation, glycation, SUMOylation, ubiquitination, Neddylation, or a combination thereof. In some embodiments, at least one of the one or more posttranslational modifications is phosphorylation. In some embodiments, the reagent comprises an antibody or fragment thereof, aptamer, or other ligand. In some embodiments, the reagent comprises a polyclonal antibody or fragment thereof. In some embodiments, the reagent comprises a monoclonal antibody or fragment thereof. In some embodiments, the reagent is a phospho-S6 240/244 antibody or fragment thereof. In some embodiments, the reagent is a phospho-S6 235/236 antibody or fragment thereof. In some embodiments, the reagent is a phospho-S6 244 antibody or fragment thereof. In some embodiments, the reagent can specifically bind to the protein at two or more sites. In some embodiments, the two or more sites can comprise at least one of the one or more posttranslational modifications. Some embodiments further comprise a peptide that decreases a binding affinity of the reagent for the protein at one or more of the two or more sites. Some embodiments further comprise a peptide that increases the specificity of the reagent for at least one of the two or more sites. In some embodiments, the peptide comprises at least one of the one or more posttranslational modifications. In some embodiments, the peptide has at least about 60%, 70%, 80%, 90%, 95%, 99%, or 100% identity to SEQ ID NO:25. In some embodiments, the protein is a ribosomal protein. In some embodiments, the protein is a large ribosomal subunit protein. In some embodiments, the protein is a small ribosomal subunit protein. In some embodiments, the protein is ribosomal protein S6. In some embodiments, the protein is ribosomal protein S6 and the one or more posttranslational modifications comprise phosphorylation on serine 235, serine 236, serine 240, serine 244, serine 247, or a combination thereof. In some embodiments, the protein is ribosomal protein S6 and at least one of the one or more posttranslational modifications is phosphorylation on serine 244. In some embodiments, the ribosomal protein S6 is a mouse protein. In some embodiments, at least one of the one or more posttranslational modifications occurs in response to a stimulus. In some embodiments, the stimulus is an environmental stimulus, a dietary or metabolic stimulus, a drug or active agent, or a toxin. In some embodiments, the stimulus is an atypical antipsychotic. In some embodiments, the stimulus is amisulpride, aripiprazole, asenapine, blonanserin, clotiapine, clozapine, iloperidone, lurasidone, mosapramine, olanzepine, paliperidone, perospirone, quetiapine, remoxipride, risperidone, sertindole, sulpiride, ziprasidone, zotepine, bifeprunox, pimavanserin, vabicaserin, or a combination thereof. In some embodiments, the heterogeneous population of cells comprises prokaryotic cells, eukaryotic cells, or a combination thereof. In some embodiments, the heterogeneous population of cells comprises mammalian cells. In some embodiments, the heterogeneous population of cells comprises mouse cells. In some embodiments, the lysate or fraction is derived from all or a portion of a cell culture. In some embodiments, the lysate or fraction is derived from a tissue sample. In some embodiments, the lysate or fraction is derived from all or a portion of an organ. In some embodiments, the lysate or fraction is derived from all or a portion of a brain, stomach, intestine, lung, or a combination thereof.

Also disclosed herein are kits for isolating actively translated mRNA from activated cells in a heterogeneous population of cells, the kits comprising: (a) an antibody or fragment thereof that binds to a single epitope of a phosphorylated S6 protein, (b) instructions for use. Also disclosed herein are kits for isolating actively translated mRNA from activated cells in a heterogeneous population of cells, the kits comprising: (a) an antibody or fragment thereof that binds to a phosphorylated S6 protein at two or more epitopes; (b) a peptide that decreases the binding affinity of the antibody or fragment thereof to one or more epitopes on the phosphorylated S6 protein; (c) instructions for use. In some embodiments, the antibody or fragment thereof is a polyclonal antibody or fragment thereof. In some embodiments, the antibody or fragment thereof is a monoclonal antibody or fragment thereof. In some embodiments, the antibody or fragment thereof is a phospho-S6 244 antibody or fragment thereof. In some embodiments, the antibody or fragment thereof is a phospho-S6 240/244 antibody or fragment thereof. In some embodiments, the antibody or fragment thereof is a phospho-S6 235/236 antibody or fragment thereof. In some embodiments, the peptide is phosphorylated. In some embodiments, the peptide has at least about 60%, 70%, 80%, 90%, 95%, 99%, or 100% identity to SEQ ID NO:25.

Also provided herein are antibodies or fragments thereof that bind to a single epitope of a phosphorylated S6 protein. In some embodiments, the antibody or fragment thereof is a monoclonal antibody or fragment thereof. In some embodiments, the antibody or fragment thereof is a polyclonal antibody or fragment thereof. In some embodiments, the single epitope comprises phosphorylated serine 244. In some embodiments, the antibody or fragment thereof does not bind to S6 protein that is not phosphorylated at serine 244.

Also provided herein are hybridomas that express a monoclonal antibody or fragment thereof that binds to a single epitope of a phosphorylated S6 protein. In some embodiments, the single epitope comprises phosphorylated serine 244.

Also provided herein are peptides that decrease the binding affinity between one or more epitopes of a phosphorylated S6 protein and an antibody or fragment thereof that binds to two or more epitopes of the phosphorylated S6 protein. In some embodiments, the peptide is phosphorylated. In some embodiments, the peptide has at least about 60%, 70%, 80%, 90%, 95%, 99%, or 100% identity to SEQ ID NO:25.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 A-E illustrates molecular anatomic profiling by pS6 capture. (A) Schematic of the approach. Cells with active mTOR signaling (grey) have ribosomes containing phosphorylated S6, and these ribosomes are captured by magnetic beads containing anti-pS6 antibodies. (B) Immunostaining for pS6 (left panels) and c-fos (middle panels) from the hippocampus of mice induced to have seizures by treatment with kainate. (C) Western blot for ribosomal proteins from wild-type or S6^{S5A} MEFs that were serum starved and restimulated with FBS plus insulin. The whole cell lysate is shown at left and the pS6 240/244 immunoprecipitate is shown at right. (D) Bioanalyzer traces of RNA associated with pS6 immunoprecipitates from wild-type and S6^{S5A} MEFs. The peaks for 18S and 28S ribosomal RNA are labeled. (E) Quantification of the RNA associated with pS6 immunoprecipitates from wild-type and S6^{S5A} MEFs (* p=0.0003).

FIG. 2 A-C illustrates the validation of pS6 235/236 immunoprecipitation. (A) Western blot for ribosomal proteins from NIH3T3 cells that were serum starved and either restimulated with FBS plus insulin or treated with rapamycin. The whole cell lysate is shown at left and the pS6 235/236 immunoprecipitate is shown at right. (B) Quantification of the RNA associated with pS6 immunoprecipitates from NIH3T3 cells restimulated with FBS plus insulin or treated with rapamycin. (C) Bioanalyzer traces of RNA associated with pS6 immunoprecipitates from the two conditions. The peaks for 18S and 28S ribosomal RNA are labeled.

FIG. 3 A-E illustrates mTORC1 activation in MCH (melanin concentration hormone) neurons. (A) GFP immunofluorescence in a brain slice from MCH^{GFP}MCH^{Cre}Tsc1^{fl/fl} mice. LH=lateral hypothalamus. Scale bar=500 Pm. (B) GFP and pS6 240/244 immunofluorescence in the LH from MCH^{GFP}MCH^{Cre}Tsc1^{fl/fl} (bottom) and MCH^{GFP}Tsc1^{fl/fl} (top) mice. (C) Quantification of soma volume in GFP positive neurons from MCH^{GFP}MCH^{Cre}Tsc1^{fl/fl} (left) and MCH^{GFP}Tsc1^{fl/fl} (right). ** p<0.0001. (D) Three dimensional reconstruction of the soma and proximal process from an MCH neuron from MCH^{GFP}MCH^{Cre}Tsc1^{fl/fl} (bottom) and MCH^{GFP}Tsc1^{fl/fl} (top) mice. Scale bar=10 Pm. (E) Quantification of fold-enrichment (IP/Input) in pS6 240/244 immunoprecipitates for mRNA for a panel neuropeptide markers. * p<0.01.

FIG. 4 A-F illustrates mTORC1 activation in hypothalamic VIP (vasoactive intestinal peptide) neurons. (A) Immunofluorescence for pS6 in the SCN of wild-type mice at baseline. (B) Scatterplot of mRNA abundance for each gene in the pS6 240/244 immunoprecipitate (IP) versus the total hypothalamic RNA (input). Selected highly enriched or depleted genes are labeled. (C) Fold-enrichment by microarray for a panel of 20 neuropeptides that mark well-characterized populations of hypothalamic neurons. (D) Fold-enrichment by Taqman for VIP mRNA in immunoprecipitates from hypothalamus in the light and dark and from the ventral cortex. (E). Percentage of VIP cells positive for pS6 in the hypothalamus in the light and dark and from the ventral cortex. (F) Immunofluoresence for pS6 and fluorescence in situ hybridization for VIP in the hypothalamus in the light (top) and dark (middle) and from the ventral cortex (bottom). *** p<0.001, **** p<0.0001.

FIG. 5 A-B illustrates a comparison between pS 240/241 and pS6 235/236 immunoprecipitations and Taqman validation. (A) Fold-enrichment by microarray for a panel of hypothalamic neuropeptides using antibodies against either pS6 235/236 or pS6 240/244. (B) Validation by Taqman of the fold-enrichment values determined by the microarray for key genes enriched or depleted in pS6 240/244 immunoprecipitates.

FIG. 6 A-D illustrates total ribosome immunoprecipitation. (A) NIH3T3 cells were serum starved for 4 h and either restimulated with 20% FBS+100 nM insulin for 30 min or treated with rapamycin for 30 min. Lysates were immunoprecipitated using a combination of antibodies against ribosomal proteins L7 and L26, and the input (left) or immunoprecipitate (right) was blotted for pS6 235/236 and total ribosomal proteins. (B) Bioanalyzer data of immunoprecipitates from panel A. (C) Fold enrichment of mRNA isolated from hypothalamic homogenates by immunoprecipitation with either pS6 240/244 (black bars) or total ribosomal antibodies (white bars) for a panel of biomarkers. (D) Fold enrichment of mRNA isolated from homogenates prepared mice that were fed or fasted overnight and immunoprecipitated with either pS6 240/244 (black bars) or total ribosomal antibodies (white bars).

FIG. 7 illustrates the relative enrichment of transcripts in pS6 immunoprecipitates from light verses dark.

FIG. 8 A-J illustrates activation of mTORC1 by fasting and leptin deficiency. (A) Distribution of fold-enrichment of genome in fasted mice relative fed controls. Agrp and Npy are the two most enriched genes. (B) Fold-enrichment of the 200 transcripts that show the greatest overall increase in hypothalamic expression in response to fasting. (C) Immunofluorescence for pS6 and AgRP-GFP in the arcuate nucleus of fasted and fed mice. (D) Quantification of the distribution of pS6 staining intensity in Agrp neurons in fasted and fed mice. (E) Mean pS6 staining intensity in Agrp neurons from fasted and fed mice. (F) Immunofluorescence for pS6 and Pomc-GFP in the arcuate nucleus of fasted and fed mice. (G) Quantification of the distribution of pS6 staining intensity in Pomc neurons in fasted and fed mice. (H) Mean pS6 staining intensity in Pomc neurons from fasted and fed mice. (I) Comparison of the fold-enrichment in pS6 immunoprecipitates for fasted or ob/ob mice versus fed controls for a panel of 20 hypothalamic neuropeptides. (J) Immunofluorescence for pS6 combined with fluorescence in situ hybridization for Npy in the arcuate nucleus of fed and ob/ob mice.

FIG. 9 A-G illustrates activation of mTORC1 by osmotic stimulation. (A) Immunofluorescence for pS6 235/236 in a brain section from mice challenged with a salt injection. Scale bar=500 Pm. (B) Distribution of fold-enrichment for genome in pS6 240/244 immunoprecipitates from osmotically challenged animals versus controls. Several highly enriched genes are labelled. (C) Co-localization of immunofluorescence for pS6 240/244 and Avp in the PVN in osmotically challenged animals and controls. Scale bar=50 Pm. (D) Quantification of the distribution of pS6 240/244 staining in Avp neurons from salt challenged animals and controls. (E) Co-localization of immunofluorescence for pS6 240/244 and FosB in the PVN in salt challenged animals and controls. Scale bar=50 Pm. (F) Quantification of percentage of pS6 positive cells in the PVN and SON of salt challenged animals that are also FosB positive. (G) Quantification of percentage of FosB positive cells in the PVN and SON of salt challenged animals that are also pS6 positive.

FIG. 10 A-B illustrates induction of pS6 in NPY neurons by fasting. (A) Immunofluorescence for pS6 235/236 in NPY-GFP labelled neurons from fed and fasted mice. (B) Quantification of distribution of pS6 intensities in NPY-GFP neurons. * p<0.01.

FIG. 11 A-B illustrates osmotic stimulation. (A) Immunofluorescence for phosphorylation of 4E-BP1 (T37/46) in the SON of mice subjected to osmotic stimulation or controls. (B) Immunofluorescence for pS6 240/244 in oxytocin neurons in the PVN from control mice and mice subjected to osmotic stimulation.

FIG. 12 A-E illustrates mTORC1 activity in oligodendrocytes. (A) Taqman for oligodendrocyte markers for pS6 immunoprecipitates from the hypothalamus and cortex. (B) Fold enrichment in immunoprecipitates from the hypothalamus at baseline for a panel of markers for neurons, oligodendrocytes, and astrocytes. *** p<0.001. (C) Three-probe imaging of neurons (“Neuron”), oligodendrocytes (“Oligo”), and pS6 (“pS6”). (D) Quantification of the density of total S6 staining (intensity/volume) for oligodendrocytes (0) and neurons (N) in four representative anatomic fields. Each data point is a cell. The mean and standard error of the S6 density are shown. The ratio of these means (neurons divided by oligodendrocytes) is calculated at the bottom. (E) Quantification of the density of pS6 240/244 staining, as described for panel D, for 8 anatomic fields that are representative of low and high pS6 regions. p<0.0001 for all comparisons within a field between the mean total S6 or mean pS6 density of oligodendrocytes versus neurons.

FIG. 13 A-I illustrates mTORC1 signaling in reticulocytes. (A) Simplified schematic of red blood cell development highlighting the loss of nucleus in reticulocytes and loss of RNA in mature red blood cells. (B) Fold-enrichment measured by Taqman for hba-a1 and hbb-b1 mRNA in pS6 240/244 immunoprecipitates from the hypothalamus in the light and dark and from the cortex. (C) Abundance of mRNA for actin, pomc, hba-a1, and hbb-b1 in hypothalamic homogenates prepared with and without prior saline perfusion. RNA quantified by Taqman and normalized to rpl23. (D) Comparison of pS6 levels in brain homogenates and RBC lysates by western blot. (E) Quantification of relative stoichiometry of pS6 in brain homogenates, RBC lysates, and RBC lysates from iron-deficient mice. (F) Mean cell volume of reticulocytes and mature RBC from mice on a standard or low iron diet. (G) Percentage of cells scored as low hemoglobin by automated counting. (H) Comparison of pS6 levels in RBC lysates from mice on a standard or low iron diet by western blotting. (J) Comparison of pS6 levels in K562 cells treated with the iron chelator DFO or DFS. * p<0.05, ** p<0.01, *** p<0.001 by two-tailed t-test.

FIG. 14 A-B illustrates a validation of ammonium chloride lysis. (A) The number of red blood cells, reticulocytes and white blood cells per mL of tail blood from normal mice. Note that the number of reticulocytes in unfractionated tail blood exceeds the number of white blood cells by ˜30:1. Also, mature red blood cells do not contain ribosomes. (B) Selective lysis of reticulocytes and mature red blood cells by ammonium chloride.

FIG. 15 illustrates a crystal structure of a ribosomal protein S6 in a ribosome subunit.

FIGS. 16 (A and B) illustrates phosphorylated ribosome profiling. A. Schematic of the approach. Activated neurons are shown in red. B. Immunostained brain slices showing co-localization of c-fos and pS6 in response to a variety of stimuli. The anatomical region magnified is indicated by the gray box below.

FIG. 17 (A-F) illustrates co-localization of pS6 and c-fos in response to a series of stimuli. A&B. Resident Intruder. C. Clozapine. D. Osmotic Stimulation. E. Olanzapine. F. Ghrelin.

FIG. 18 illustrates co-localization of pS6 and c-fos in the SCN following light stimulation (45 min) at the end of the dark phase. Inset region is shown in the third row.

FIG. 19 (A-E) illustrates selective capture of phosphorylated ribosomes. A. Western blot for ribosomal proteins from wild-type or S6^{S5A} MEFs. The whole cell lysate is shown at left and the pS6 240/244 immunoprecipitate is shown at right. B. Quantification of RNA associated with pS6 immunoprecipitates from wild-type and S6^{S5A} MEFs. C. Bioanalyzer analysis of immunoprecipitated RNA from wild-type and S6^{S5A} MEFs. The peaks for 18S and 28S ribosomal RNA are labeled. D. Co-localization of MCH and pS6 in the lateral hypothalamus of Tsc1^{fl/fl} and MCH^{Cre} Tsc1^{fl/fl}. E. Enrichment of cell-type specific genes in pS6 immunoprecipitates from Tsc1^{fl/fl} and MCH^{Cre} Tsc1^{fl/fl} mice. Data determined by Taqman and normalized to rpL27.

FIG. 20 (A-B) illustrates microarray scatter plots of RNA in pS6 240/244 immunoprecipitate versus total RNA from A. Hepa1-6 cells and B. NIH3T3 cells.

FIG. 21 (A-C) illustrates enhanced selectivity via synthetic antibodies that target pS6 244. A. Schematic of S6 phosphorylation sites, their recognition by commercially available phosphospecific antibodies, and the 3P peptide used to alter antibody specificity. B. Fold-enrichment for MCH neuron specific markers in immunoprecipitates using a pS6 240/244 polyclonal antibody with and without prior addition of the 3P peptide. C. Adjacent sections from the hypothalamus of a wild-type mouse stained with a pS6 240/244 antibody in the presence (bottom) or absense (top) of the 3P peptide.

FIG. 22 (A-G) illustrates identification of neurons activated by salt challenge. A. Hypothalamic staining for pS6 244 from mice given an injection of vehicle (PBS) or 2M NaCl. Scale bar=200 μm B. Differential enrichment of cell-type specific genes in pS6 immunoprecipitates. Data are expressed as the ratio of fold-enrichment (IP/input) for salt-treated animals divided by the fold-enrichment (IP/input) for controls and plotted on a log-scale. Key genes are labeled. C. Co-localization between Avp, Oxt, and Crh with pS6 in salt-treated and control animals. Crh neurons were analyzed as two separate populations in the rostral and caudal PVN. D. Quantification by confocal imaging of pS6 intensity within individual Avp, Oxt, and Crh neurons from salt-treated and control animals. E. Co-localization between FosB, Cxcl1 and pS6 in salt-treated and control animals. F. Percentage of FosB positive cells in the PVN and SON that are also pS6 positive. G. Percentage of pS6 positive cells in the PVN and SON that are also FosB positive.

FIG. 23 (A-D) A Immunohistochemical co-localization of Sim1 and pS6 in the hypothalamus of salt-challenged animals. B. Analysis of RNA by Illumina microarray in pS6 immunoprecipitates of osmotically stimulated animals relative to controls. Key genes are labeled. Note that this earlier experiment was not performed using the 3P peptide, which is reflected in the lower fold-enrichment values. This data is shown to illustrate that the rank-order of the most highly enriched genes is the same as observed by Taqman. C. RNA-seq analysis of RNA in pS6 immunoprecipitates of osmotically stimulated animals relative to controls. Key genes are labeled, indicating that the most highly enriched genes show extensive overlap with the most highly enriched genes measured by Taqman. D. Fold enrichment of each gene in total ribosome immunoprecipitates of osmotically stimulated animals relative to controls. 225 probes from the Taqman array (Table 4) are shown and plotted on the same scale as FIG. 22b. No genes show >2-fold enrichment.

FIG. 24 (A-F) illustrates identification of neurons activated by fasting. A. Hypothalamic staining for pS6 244 from fasted and fed mice. Scale bar=100 μm B. Relative enrichment of cell-type specific genes in pS6 immunoprecipitates from fasted and fed animals. Data are expressed as the ratio of fold-enrichment (IP/input) for fasted animals divided by the fold-enrichment (IP/input) for fed controls and plotted on a log-scale. Key genes are labeled. C. Co-localization between AgRP and pS6 in fed and fasted mice. (Right). Quantification of pS6 intensity in AgRP neurons. D. Co-localization between POMC and pS6 in fed and fasted mice. (Right) Quantification of pS6 intensity in POMC neurons. E. Co-localization between GAL and pS6 in fed and fasted mice in the MPA and DMH. F. Co-localization between GAL and c-fos in fed and fasted mice in the MPA and DMH.

FIG. 25 (A-H) illustrates pS6 immunostaining of consecutive hypothalamic sections from mice that were fasted or fed ad libitum and sacrificed at the end of the dark phase. Key regions that show enhanced pS6 in response to fasting are labeled. A. is the most Rostral section. H. is the most Caudal section.

FIG. 26 Top. Co-localization between galanin and GAD67-GFP in the DMH. Bottom: Absense of co-localization between galanin and ObRb-GFP in the DMH.

FIG. 27 (A-J) illustrates identification of neurons activated by ghrelin and scheduled feeding. A. Hypothalamic staining for pS6 in response to ghrelin (IP injection, 1 h) or scheduled feeding (2 h following food presentation). Scale bar=100 μm B. Time course of pS6 staining in the DMH in mice acclimated to a protocol of scheduled feeding between circadian time (CT) 4-7. Mice were either fed (top) or not fed (bottom) on the day of the experiment. Scale bar=50 μm C. Quantification of the number of pS6 positive cells in the DMH (left) and Arc (right) in mice on a scheduled feeding protocol. Black indicates mice that were fed on the day of the experiment; gray indicates mice that were not fed. D. Differential enrichment of cell-type specific transcripts in pS6 IPs from mice that were given ghrelin (y-axis) or subjected to scheduled feeding (x-axis). Data are expressed as the ratio of fold-enrichment (IP/input) from ghrelin or scheduled feeding animals relative to the fold-enrichment of their controls and plotted on a log-scale. Key genes are labeled. E. Expression of Pdyn in the hypothalamus and its co-localization with pS6 in subjected to scheduled feeding and sacrificed at CT6. Note the overlap between Pdyn and pS6 in the DMH but not the Arc. F. Quantification of co-localization between Pdyn and pS6 in various hypothalamic nuclei of mice fed ad libitum or subjected to scheduled feeding and sacrificed at CT6. G. Co-localization between Pdyn and pS6 in the DMH of ad libtum and scheduled feeding. Scale bar 50 m. H. Food intake (top) and change in body weight (bottom) of mice given an intraperitoneal injection of the KOR antagonist JDTic (gray) or vehicle (black). Mice were switched from ad libitium to scheduled feeding on day 0. I. Food intake (top) and change in body weight (bottom) for mice given an intraperitoneal injection of the KOR antagonist JDTic (gray) or vehicle (black) and maintained on an ad libitum diet. J. Food intake (top) and change in body weight (bottom) of mice given an intracerebroventricular injection of the KOR antagonist norbinaltorphimine or vehicle (black). Mice were switched from ad libitium to scheduled feeding on day 0.

FIG. 28 (A-C) A. Co-localization between NPY and pS6 in ad libitum, ghrelin-treated, and scheduled feeding mice. Scale bar=100 μm B. Co-localization between Pdyn and c-fos at CT6 in mice subjected to scheduled feeding. C. Mice were preacclimated to a scheduled feeding protocol and then given an injection of JDTic (5 μL at 1 mg/mL) into the lateral ventricle on Day 0 and food intake was recorded. Note that unlike experiments in FIG. 27, mice in this experiment were acclimated to the scheduled feeding protocol for two weeks prior to drug injection. Therefore the decline in food intake on Day 0 was transient and reflected the effect of surgery not a change in feeding protocol.

FIG. 29 illustrates in situ hydribidization data from the Allen Brain Atlas for Gpr50, Gsbs, Pdyn, and Npvf.

### DETAILED DESCRIPTION OF THE INVENTION

### Translational Profiling and Molecular Phenotyping

The present disclosure provides for methods, compositions, and kits useful in translational profiling and molecular phenotyping of subpopulations of heterogeneous tissues and cell populations. The methods disclosed herein can be used to identify mRNA whose translation is modulated by a stimulus. The stimulus can be an environmental stimulus. The stimulus can be a metabolic or dietary stimulus. The stimulus can be a drug, therapeutic agent, or other active agent. The stimulus can be a toxin and/or a carcinogen.

The methods, compositions, and kits disclosed herein can be used to identify one or more cell types in a subpopulation of cells within heterogeneous tissues and/or cell populations that are responding to a stimulus. A cell, cell type, or tissue responding to a stimulus can be termed an activated cell, cell type, or tissue. A cell, cell type, or tissue responding to a stimulus (e.g., an activated cell) can have altered activity in one or more signaling pathways. A cell, cell type, or tissue responding to a stimulus (e.g., an activated cell) can have a greater percentage of one or more proteins that are posttranslationally modified. The one or more proteins can be ribosomal proteins.

Translational profiling can be the profiling, identification, quantitation, or isolation of actively translated mRNAs. Such profiling can be a measure of the nascent proteome. Translational profiling can allow for the identification of mRNAs being actively translated or otherwise associated with the cellular translational machinery. Translational profiling, according to the methods disclosed herein, can allow for the identification of mRNAs whose translation is modulated by a stimulus. Molecular phenotyping can be the molecular and/or gene expression description of organs, tissues, or cell types; for example, organs, tissues, or cells that are responding to a stimulus.

The present disclosure provides for methods and compositions to practice translating ribosome affinity purification (TRAP) profiling methodology. These profiling methods can be utilized to further distinguish morphologically, anatomically, developmentally, or otherwise indistinguishable, cells into cellular subtypes, further defining cell populations and sub-populations. In some cases, these otherwise indistinguishable cells are intermixed. In other cases, these cells are spatially separated. In some cases, these cells are cells of the central or peripheral nervous system, for example neurons or glia. In some cases, these cells can be distinguishable by their translational profiles and molecular phenotypes. In other cases these are cells outside the nervous system.

The methods provided herein allow for isolation of mRNAs associated with ribosomes or polysomes (clusters of ribosomes) from subpopulations of cells responding to a stimulus, allowing for translational profiling and molecular phenotyping of the cell, tissue, or organism response to the stimulus. In some embodiments, the mRNA is targeted by a reagent that specifically binds to a protein associated with a ribosome (e.g., a ribosomal protein). In some embodiments, the protein associated with the ribosome (e.g., a ribosomal protein) is posttranslationally modified in response to the stimulus. In some embodiments, the reagent specifically binds to the posttranslationally modified protein. In some embodiments, the reagent specifically binds to the posttranslationally modified protein at one or more sites of posttranslational modification. In some embodiments, the reagent has a decreased affinity or substantially no affinity for the protein without the posttranslational modification. Specific or selective binding can be defined as binding that is not competed away by addition of non-specific proteins (e.g., bovine serum albumen (BSA)).

The methods described herein in allow for identifying actively translated mRNA in any cell subtype of interest. The methods disclosed herein allow for identifying mRNA whose translation is modulated by any stimulus of interest. The methods disclosed herein can involve the isolation or purification of intact ribosomes or polysomes. In some embodiments, the purification of ribosomes or polysomes is by affinity or immunoaffinity purification.

### Ribosomal Proteins

Disclosed herein are methods, compositions, and kits for isolating ribosomes, ribosomal complexes, or polysomes and their associated mRNA. As used herein, the term ribosome is meant to encompass ribosomal complexes and polysomes. The ribosome can be actively translating the mRNA. An mRNA associated with a ribosome or actively translating ribosome can be referred to herein as an actively translated mRNA.

A ribosome can be a large ribonucleoprotein particle comprising both protein and RNA components. Ribosomes can vary in size and structure between the three domains of life: bacteria, archaea, and eukaryotes. Ribosomes can be described as having two subunits: a large subunit and a small subunit. Ribosomes, ribosome subunits, ribosomal RNAs, and ribosomal proteins can be identified according to a Svedberg (S) unit, which can be a measure of the rate of sedimentation in centrifugation. In bacteria, ribosomes subunits can be referred to as the 30S subunit and the 50S subunit; assembled, bacterial ribosomes can be referred to as 70S ribosomes. The small or 30S subunit of a bacterial ribosome can comprise a 16S RNA molecule bound to about 21 proteins; the large or 50S subunit of a bacterial ribosome can comprise a 5S RNA molecule, a 23S RNA molecule, and 31 proteins. Eukaryotic ribosomes can comprise a 40S subunit and a 60S subunit; assembled, a Eukaryotic ribosome can be referred to as an 80S ribosome. The small or 40S subunit of a Eukaryotic ribosome can comprise an 18S RNA molecule and 33 proteins; the large or 60S subunit can comprise 5S RNA, a 28S RNA, a 5.8S RNA and about 49 proteins.

There are many families of ribosomal proteins. The naming convention for ribosomal protein families can be a letter, either L or S, which can identify whether the protein is associated with the large or small ribosomal subunit; followed by a number, which can identify the ribosomal protein according to rate of sedimentation in centrifugation in Svedberg units. Individual ribosomal proteins can have the same name as the ribosomal protein family to which they belong; however, many ribosomal proteins have alternative names as well. A ribosomal protein can be a member of the S1p, S2p, S3p, S4p, S5p, S6p, S7p, S8p, S9p, S10p, S11p, S12p, S13p, S14p, S15p, S16p, S17p, S18p, S19p, S20p, S21p, S22p, S3ae, S4e, S6e, S7e, S8e, S10e, S12e, S17e, S19e, S21e, S24e, S25e, S26e, S27ae, S27e, S28e, S30e, S31e, L1p, L2p, L3p, L4p/L4e, L5p, L6p, L9p, L10p, L11p, L12p, L13p, L14p, L15p, L16p, L17p, L18p, L19p, L20p, L21p, L22p, L23p, L24p, L25p, L27p, L28p, L29p, L30p, L31p, L32p, L33p, L34p, L35p, L36p, L6e, L7ae, L10e, L13e, L14e, L15e, L18ae, L18e, L19e, L21e, L22e, L24e, L27e, L28e, L29e, L30e, L31e, L32e, L34e, L35ae, L36e, L37ae, L37e, L38e, L39e, L40e, L41e, L44e, or LXa ribosomal protein family.

Ribosomes and their associated mRNA can be isolated from a lysate or fraction of a heterogeneous population of cells using a reagent that specifically binds to a protein associated with the ribosome. The protein can be a ribosomal protein. The ribosomal protein can be a large ribosomal subunit protein; for example, the ribosomal protein can be a L1p, L2p, L3p, L4p/L4e, L5p, L6p, L9p, L10p, L11p, L12p, L13p, L14p, L15p, L16p, L17p, L18p, L19p, L20p, L21p, L22p, L23p, L24p, L25p, L27p, L28p, L29p, L30p, L31p, L32p, L33p, L34p, L35p, L36p, L6e, L7ae, L10e, L13e, L14e, L15e, L18ae, L18e, L19e, L21e, L22e, L24e, L27e, L28e, L29e, L30e, L31e, L32e, L34e, L35ae, L36e, L37ae, L37e, L38e, L39e, L40e, L41e, L44e, or LXa ribosomal family protein. The ribosomal protein can be a small ribosomal subunit protein; for example, the ribosomal protein can be a S1p, S2p, S3p, 54p, S5p, S6p, S7p, S8p, S9p, S10p, S11p, S12p, S13p, S14p, S15p, S16p, S17p, S18p, S19p, S20p, S21p, S22p, S3ae, S4e, S6e, S7e, S8e, S10e, S12e, S17e, S19e, S21e, S24e, S25e, S26e, S27ae, S27e, S28e, S30e, S31e ribosomal family protein. Exemplary ribosomal proteins for use in the methods disclosed herein are provided in Table 1, but are not limited to those listed. In an exemplary embodiment, the ribosomal protein is S6. The ribosomal protein can be incorporated into a ribosomal complex, ribosome, or polysome. The ribosomal complex, ribosome, or polysome can be associated with mRNA. In some embodiments, the ribosomal protein does not bind mRNA directly.

TABLE 1
Ribosomal Proteins

| | | | | | | |
|---|---|---|---|---|---|---|
| A52 | L11 | L23a | L35a | LP2 (Large P2) | S11 | S24 |
| Ke-3 | L12 | L24 | L36 | LP1 (Large P1) | S12 | S25 |
| L3 | L13 | L26 | L36a | S2 | S13 | S26 |
| L3L (L3- | L13a | L27 | L37 | S3 | S14 | S27 |
| like) | | | | | | |
| L4 | L14 | L27a | L37a | S3a | S15 | S27a |
| L5 | L15 | L28 | L38 | S4 | S15a | S28 |
| L6 | L17 | L29 | L39 | S5 | S16 | S29 |
| L7 | L18 | L30 | L41 | S6 | S17 | S30 |
| L7a | L18a | L31 | L44 | S7 | S18 | S23 |
| L8 | L19 | L32 | LAMR1 | S8 | S19 | RPLP1 |
| L9 | L21 | L32-3a | LLRep3 | S9 | S20 | |
| | | (3a) | | | | |
| L10 | L22 | L34 | LP0 | S10 | S21 | |
| | | | (Large P0) | | | |

| | | | |
|---|---|---|---|
| L10a | L23 | L35 | Region containing hypothetical |
| | | | protein FLJ23544 |

The protein can be ribosomal protein S6, which can also be referred to as S6, RPS6, Phosphoprotein NP33, or 40S Ribosomal Protein S6. Encoding nucleotide and peptide sequences for exemplary ribosomal protein S6 proteins that can be used in the methods and compositions disclosed herein can include, but are not limited to, those found in Table 2.

TABLE 2
Ribosomal Protein S6 Sequences

| | |
|---|---|
| SEQ ID NO: 5 | MKLNISFPATGCQKLIEVDDERKLRTFYEKRMATEVAA |
| GenBank: AAH92050.1 | DALGEEWKGYVVRISGGNDKQGFPMKQGVLTHGRVR |
| Mus musculus | LLLSKGHSCYRPRRTGERKRKSVRGCIVDANLSVLNLVI |
| Ribosomal Protein S6 | VKKGEKDIPGLTDTTVPRRLGPKRASRIRKLFNLSKEDD |
| Peptide Sequence | VRQYVVRKPLNKEGKKPRTKAPKIQRLVTPRVLQHKR |
| | RRIALKKQRTKKNKEEAAEYAKLLAKRMKEAKEKRQE |
| | QIAKRRRLSSLRASTSKSESSQK |
| SEQ ID NO: 6 | cgcctcccaggcgctcggctgtgtcaagatgaagctgaacatctccttccccgccaccg |
| GenBank: BC092050.1 | gctgtcagaagctcatcgaggtggatgacgagcgcaagctccgcaccttctatgagaa |
| Mus musculus | gcgcatggccacggaagtagccgctgatgctcttggtgaagagtggaagggttatgtg |
| Ribosomal Protein S6 | gtccggatcagcggtgggaatgacaagcaaggttttcccatgaagcaaggtgttctgac |
| Nucleotide Sequence | ccatggcagagtgcgcctgctgttgagtaaggggcattcctgttacaggccaaggaga |
| mRNA | actggagagaggaagcgcaagtctgttcgtggatgcattgtggacgctaatctcagtgtt |
| | ctcaacttggtcattgtaaagaaaggagagaaggatattcctggactgacagacactact |
| | gtgcctcgtcggttgggacctaaaagggctagtagaatccgcaagctttttaatctctcca |
| | aagaagatgatgtccgccagtatgttgtcaggaagcccttaaacaaagaaggtaagaag |
| | cccaggaccaaagcacccaagattcagcgacttgttactcctcgtgtcctgcaacacaa |
| | acgccgacgtattgctctgaagaagcaacgcactaagaagaacaaggaggaggctgc |
| | agaatacgctaaacttttggccaagagaatgaaggaagccaaagaaaagcgccagga |
| | acagattgccaagagacgtaggctgtcctcactgagagcttctacttctaagtctgagtcc |
| | agtcaaaaatgagtctttaagagcaacaaataaataatgaccttgaatctttaaaaaaaaa |
| | aaaaaaaaaaaaaaa |
| SEQ ID NO: 7 | MKLNISFPATGCQKLIEVDDERKLRTFYEKRMATEVAA |
| GenBank: AAA42079.1 | DALGEEWKGYVVRISGGNDKQGFPMKQGVLTHGRVR |
| Rattus norvegicus | LLLSKGHSCYRPRRTGERKRKSVRGCIVDANLSVLNLVI |
| Ribosomal Protein S6 | VKKGEKDIPGLTDTTVPRRLGPKRASRIRKLFNLSKEDD |
| Peptide Sequence | VRQYVVRKPLNKEGKKPRTKAPKIQRLVTPRVLQHKR |
| | RRIALKKQRTKKNKEEAAEYAKLLAKRMKEAKEKRQE |
| | QIAKRRRLSSLRASTSKSESSQK |
| SEQ ID NO: 8 | gtcggctgtgtcaagatgaagctgaatatctccttccctgccactggctgtcagaaactca |
| GenBank: M29358.1 | tagaagtggatgacgaacgcaagcttcgtacgttctatgagaagcgcatggccacaga |
| Rattus novegicus | aaatgacaaacaaggttttcccatgaagcaaggcgttttgacccatggcagagtgcgcc |
| Ribosomal Protein S6 | tgcttttgagtaaggggcattcttgttatagacctaggagaactggagagaggaagcgca |
| Nucleotide Sequence | agtctgtccgaggatgcattgtggatgccaacctgagtgttctcaacttggttattgtaaaa |
| mRNA | aaaggagagaaggatattccaggactgacagataccactgtgcctcgtcggttgggac |
| | ctaaaagagctagtagaatccgaaagctttttaatctctccaaagaagatgatgtccgcca |
| | gtatgttgttagaaagcccttaaacaaagaaggtaagaagcccaggaccaaagcgccc |
| | aagattcagcgtcttgttactccccgtgtcctgcaacacaaacgccgacgtattgctctga |
| | agaagcaacgcactaagaaaaacaaggaggaggctgcagaatatgctaaacttttggc |
| | caagagaatgaaggaagccaaagagaagcgccaggaacagattgccaagagacgta |
| | ggctgtcttcgctgagagcttctacttctaaatctgagtccagtcaaaaataagtctttaaa |
| | gagtaacaaataaataatgagaccttg |
| SEQ ID NO: 9 | MKLNISFPATGCQKLIEVDDERKLRTFYEKRMATEVAA |
| NCBI: XP_003339215.1 | DALGEEWKGYVVRISGGNDKQGFPMKQGVLTHGRVR |
| Pan troglodytes | LLLSKGHSCYRPRRTGERKRKSVRGCIVDANLSVLNLVI |
| 40S Ribosomal Protein | VKKGEKDIPGLTDTTVPRRLGPKRASRIRKLFNLSKEDD |
| S6 | VRQYVVRKPLNKEGKKPRTKAPKIQRLVTPRVLQHKR |
| Peptide Sequence | RRIALKKQRTKKNKEEAAEYAKLLAKRMKEAKEKRQE |
| | QIAKRRRLSSLRASTSKSESSQK |
| SEQ ID NO: 10 | tcgcgagaactgaaagcgcctatgtgacctgcgctaagcggaagttggcccttttttccg |
| NCBI: | tggcgcctcggaggcgttcagctgcttcaagatgaagctgaacatctccttcccagcca |
| XM_003339167.1 | ctggctgccaaaaactcattgaagtggacgatgaacgcaaacttcgtactttttatgagaa |
| Pan troglodytes | gcgtatggccacagaagttgctgctgacgctctgggtgaagaatggaagggttatgtgg |
| 40S Ribosomal Protein | tccgaatcagtggtgggaacgacaaacaaggtttccccatgaagcagggtgtcttgacc |
| S6 | catggccgtgtccgcctgctactgagtaaggggcattcctgttacagaccaaggagaac |
| Nucleotide Sequence | tggagaaagaaagagaaaatcagttcgtggttgcattgtggatgcaaatctgagcgttct |
| mRNA | caacttggttattgtaaaaaaaggagagaaggatattcctggactgactgatactacagtg |
| | cctcgccgcctgggccccaaaagagctagcagaatccgcaaacttttcaatctctctaaa |
| | gaagatgatgtccgccagtatgttgtaagaaagcccttaaataaagaaggtaagaaacct |
| | aggaccaaagcacccaagattcagcgtcttgttactccacgtgtcctgcagcacaaacg |
| | gcggcgtattgctctgaagaagcagcgtaccaagaaaaataaagaagaggctgcaga |
| | atatgctaaacttttggccaagagaatgaaggaggctaaggagaagcgccaggaacaa |
| | attgcgaagagacgcagactttcctctctgcgagcttctacttctaagtctgaatccagtca |
| | gaaataagattttttgagtaacaaataaataagatcagactcggatctctacaaaaaaaag |
| SEQ ID NO: 11 | MKLNISFPATGCQKLIEVDDERKLRTFYEKRMATEVAA |
| GenBank: AAH13296.1 | DALGEEWKGYVVRISGGNDKQGFPMKQGVLTHGRVR |
| Homo sapiens | LLLSKGHSCYRPRRTGERKRKSVRGCIVDANLSVLNLVI |
| Ribosomal Protein S6 | VKKGEKDIPGLTDTTVPRRLGPKRASRIRKLFNLSKEDD |
| Peptide Sequence | VRQYVVRKPLNKEGKKPRTRAPKIQRLVTPRVLQHKRR |
| | RIALKKQRTKKNKEEAAEYAKLLAKRMKEAKEKRQEQ |
| | IAKRRRLSSLRASTSKSESSQK |
| SEQ ID NO: 12 | ctcggaggcgttcagctgcttcaagatgaagctgaacatctccttcccagccactggctg |
| GenBank: BC013296.2 | ccagaaactcattgaagtggacgatgaacgcaaacttcgtactttctatgagaagcgtat |
| Homo sapiens | ggccacagaagttgctgctgacgctctgggtgaagaatggaagggttatgtggtccgaa |
| Nucleotide Sequence | tcagtggtgggaacgacaaacaaggtttccccatgaagcagggtgtcttgacccatggc |
| mRNA | cgtgtccgcctgctactgagtaaggggcattcctgttacagaccaaggagaactggaga |
| | aagaaagagaaaatcagttcgtggttgcattgtggatgcaaatctgagcgttctcaacttg |
| | gttattgtaaaaaaaggagagaaggatattcctggactgactgatactacagtgcctcgc |
| | cgcctgggccccaaaagagctagcagaatccgcaaacttttcaatctctctaaagaagat |
| | gatgtccgccagtatgttgtaagaaagcccttaaataaagaaggtaagaaacctaggac |
| | cagagcacccaagattcagcgtcttgttactccacgtgtcctgcagcacaaacggcggc |
| | gtattgctctgaagaagcagcgtaccaagaaaaataaagaagaggctgcagaatatgct |
| | aaacttttggccaagagaatgaaggaggctaaggagaagcgccaggaacaaattgcg |
| | aagagacgcagactttcctctctgcgagcttctacttctaagtctgaatccagtcagaaat |
| | aagattttttgagtaacaaataaataagatcagactctgaaaaaaaaaaaaaaaaaaaaa |
| | a |
| SEQ ID NO: 13 | MKLNISFPATGCQKLIEVDDERKLRTFYEKRMATEVAA |
| GenBank: AAX09042.1 | DALGEEWKGYVVRISGGNDKQGFPMKQGVLTHGRVR |
| Bos taurus | LLLSKGHSCYRPRRTGERKRKSVRGCIVDANLSVLNLVI |
| Ribosomal Protein S6 | VKKGEKDIPGLTDTTVPRRLGPKRASRIRKLFNLSKEDD |
| Peptide Sequence | VRQYVVRKPLNKDGKKPRTKAPKIQRLVTPRVLQHKR |
| | RRIALKKQRTKKNKEEAAEYAKLLAKRMKEAKEKRQE |
| | QIAKRRRLSSLRASTSKSESSQK |
| SEQ ID NO: 14 | gcgcctcggaggctgtcggccgcttcagaatgaagctgaacatctctttcccggccact |
| GenBank: BT021025.1 | ggctgccagaagctcattgaagtggacgatgaacgaaaacttcgtaccttctacgagaa |
| Bos Taurus | gcgtatggccacagaagttgctgctgacgctctgggtgaagaatggaagggttatgtgg |
| Ribosomal Protein S6 | tccgaatcagtggcgggaacgataagcagggtttccccatgaagcagggtgtcttgacc |
| Nucleotide Sequence | catggcagagttcgcctgctactgagtaaggggcattcctgttacagaccaaggaggac |
| mRNA | tggagagagaaagcgcaaatctgtacggggttgcattgtggatgccaatctgagtgttct |
| | caatttggtcatcgtgaaaaaaggggaaaaggatattcctggactcactgatactacagt |
| | gcctcgtcgcctgggtcccaaaagagccagcagaatccgcaaacttttcaatctctctaa |
| | agaagatgatgtccgccaatatgttgtgcgaaagcccctaaacaaagacggtaagaaa |
| | cctaggactaaagcacccaagattcagcgtctcgtgactccacgagttctgcagcacaa |
| | acgccggcgtattgctctgaagaaacagcgtactaagaaaaataaagaagaggctgca |
| | gaatatgctaaacttttggccaagagaatgaaggaggccaaagaaaaacggcaggaa |
| | cagattgccaagagacggaggctgtcctctctgagagcttctacttctaagtctgagtcca |
| | gtcaaaaatgagatgttctaagagtaacaaataaataagatcagacatc |
| SEQ ID NO: 15 | MKLNISFPATGCQKLIEVDDERNVRTFYEKRMATEVAA |
| GenBank: CAA57493.1 | DSLGEEWKGYVVRISGGNDKQGFPMKQGVLTHGRVRL |
| Gallus gallus | LLSKGHSCYRPRRTGERKRKSVRGCIVDANLSVLNLVI |
| Ribosomal Protein S6 | VKKGEKDIPGLTDTTVPRRLGPKRASRIRKLFNLSKEDD |
| Peptide Sequence | VRQYVVRKPLNKEGKKPRTKAPKIQRLVTPRVLQHKR |
| | RRIALKKQRTQKNKEEAADYAKLLAKRMKEAKEKRQE |
| | QIAKRRRLSSLRASTSKSESSQK |
| SEQ ID NO: 16 | ccggcgcagttcggcgaggatgaagctcaacatctctttcccagccactggctgccaga |
| GenBank: X81968.1 | agcttattgaagtggatgatgagcgcaacgtgagaacattctatgagaagcgaatggcc |
| Gallus gallus | acggaggttgcggctgattctcttggcgaggagtggaagggctatgttgtccggatcag |
| Ribosomal Protein S6 | tggtggcaatgataaacaaggcttccccatgaagcagggtgtccttactcatggacgtgt |
| Nucleotide Sequence | ccgccttctgctcagcaaaggccactcctgctaccgccccaggagaactggagagaga |
| mRNA | aaacgcaagtctgttcggggttgcattgttgacgccaacttgagtgttctgaacttggtcat |
| | tgtgaaaaagggtgagaaggatattcctgggctgacagacacaactgtgcctcgtcgtc |
| | ttggtcccaagagagctagcaggatccgcaagctgttcaatctctctaaggaagatgatg |
| | ttcgccagtatgttgtgaggaaacctctgaataaagagggcaagaaacccaggaccaa |
| | ggctcctaagatccagcgactagtgactcctcgtgttctgcaacataagcgcagacgtat |
| | tgccctgaagaagcagcgcactcagaagaacaaggaggaggcagcagattacgcga |
| | agctcttggcaaagagaatgaaggaggccaaggaaaaacgccaggagcagattgcg |
| | aagagacgcaggctttcttcattgagagcttctacatctaaatctgagtcaagtcagaagt |
| | aaagatgtacatgatactgaaaataaaacccttttgtggttaaattttactgtgagacttcca |
| | gtgaatatatttcctggctatgtcttaaaataaatggtagtccagactaaaaaaaaaaaaaa |
| | aa |
| SEQ ID NO: 17 | MKLNISFPATGCQKLIEVDDERKLRTFYEKRMATEVAA |
| NCBI: XP_002710947.1 | DALGEEWKGYVVRISGGNDKQGFPMKQGVLTHGRVR |
| Oryctolagus cuniculus | LLLSKGHSCYRPRRTGERKRKSVRGCIVNANLSVLNLVI |
| Ribosomal Protein S6- | VKKGEKDIPGLTDTTVPRRLGPKRASRIRKLFNLSKEDD |
| like | VRQYVVRKPLNKEGKKPRTKAPKIQRLVTPRVLQHKR |
| Peptide Sequence | RRIALKKQRTKKNKEEAAEYAKFLAKRMKEAKEKRQE |
| | QIAKRCRLSSLRASTSKSESSQK |
| SEQ ID NO: 18 | gcgcctccgagccggtcagctgcttcaaaatgaagctgaatatctccttcccagccactg |
| NCBI: | gctgccagaaactcatcgaagtggacgatgaacgtaaacttcgtactttctatgagaagc |
| XM_002710901.1 | gtatggccacagaagttgctgccgatgctctgggtgaagaatggaagggttatgtggtc |
| Oryctolagus cuniculus | cggatcagtggtgggaatgataaacaaggttttcccatgaagcaaggtgtcttgacccat |
| Ribosomal Protein S6- | gggcgggtccgcctgctgctgagtaaggggcattcctgttacagaccaaggagaactg |
| like | gagaaagaaagcgcaaatcagttcggggctgcattgtcaatgccaatttgagtgttctca |
| Nucleotide Sequence | acttggttattgtaaaaaaaggagagaaagatattcctggattgactgataccacggtgcc |
| mRNA | tcgtcgcctgggtcctaaaagagccagcagaattcgtaaacttttcaatctttctaaagaa |
| | gatgatgtacgccagtatgttgtaagaaagcccttaaacaaagaaggtaagaaacctag |
| | gaccaaagcacccaagattcagcgtctggttactccacgtgtcctgcaacacaaacgcc |
| | ggcgaattgctctgaagaaacagcgtactaagaagaacaaggaggaggctgcagaat |
| | atgctaaattcttggccaagagaatgaaggaggccaaagaaaaacgccaggaacaaat |
| | tgccaagagatgtaggctgtcttctctgagagcgtctacttctaaatctgagtccagtcaa |
| | aaataaggtttaatgacaacaaataaataagattgtgtttcagatctcctttaaaaaaaataa |
| | taat |
| SEQ ID NO: 19 | MKLNISFPATGCQKLIEVEDERKLRTFYEKRMATEVAA |
| NCBI: NP_989152.1 | DPLGDEWKGYVVRISGGNDKQGFPMKQGVLTHGRVR |
| Xenopus tropicalis | LLLSKGHSCYRPRRTGERKRKSVRGCIVDANLSVLNLVI |
| Ribosomal Protein S6 | VRKGEKDIPGLTDNTVPRRLGPKRASRIRKLFNLSKEDD |
| Peptide Sequence | VRQYVVRKPLAKEGKKPRTKAPKIQRLVTPRVLQHKR |
| | RRIALKKQRTQKNKEEASEYAKLLAKRTKEAKEKRQE |
| | QIAKRRRLSSLRASTSKSESSQK |
| SEQ ID NO: 20 | gggggatctaagacagactggttgttggccatgaagcttaacatctccttcccagccact |
| NCBI: NM_203821.1 | ggctgccaaaagctcatcgaagtggaggatgagcgcaagctgcgtaccttctatgaga |
| Xenopus tropicalis | agcgcatggctacagaggttgctgcagatcccttgggtgatgagtggaagggatatgtc |
| Ribosomal Protein S6 | gttcgcatcagcggtggaaatgataagcaaggctttcccatgaaacagggagtgctaac |
| Nucleotide Sequence | tcatggccgtgttcgtcttctgttgagcaagggtcattcctgttatcgccccaggaggact |
| mRNA | ggtgaacgcaagcgcaagtctgttcgtgggtgtattgtggatgctaacctgagtgtcctg |
| | aacttggttattgttaggaaaggcgagaaggatattcctggacttacagacaacactgttc |
| | ctcgtcgcctgggtcccaaaagagccagcagaatccgcaaactgttcaacttgtcaaaa |
| | gaagatgatgtgcgtcaatatgtagtgaggaagcctctggctaaggaggggaagaagc |
| | ccaggaccaaggcccctaaaatccagcgtctagtgaccccgagagttctgcagcacaa |
| | gcgcagacgtattgctttgaagaagcagcgcactcagaagaataaggaagaggcatca |
| | gagtatgctaaacttctggctaagagaacaaaggaagccaaggaaaaacgccaggag |
| | caaattgccaagaggcgcagactgtcttctttgagagcctccacatccaaatctgaatcg |
| | agtcagaaataaaactccatcatgtaaaaataaatacattttgttgtaaacttaaaaaaaaa |
| | aaaaaaaaaaaaaaaa |
| SEQ ID NO: 21 | MKLNISFPATGCQKLIEVEDERKLRTFYEKRMATEVAA |
| NCBI: NP_001080589.1 | DPLGDEWKGYVVRISGGNDKQGFPMKQGVLTHGRVR |
| Xenopus laevis | LLLSKGHSCYRPRRTGERKRKSVRGCIVDANLSVLNLVI |
| 40S Ribosomal Protein | VRKGEKDIPGLTDNTVPRRLGPKRASRIRKLFNLSKEDD |
| S6 | VRQYVVRKPLAKEGKKPRTKAPKIQRLVTPRVLQHKR |
| Peptide Sequence | RRIALKKQRTQKNKEEASEYAKLLAKRSKEAKEKRQE |
| | QIAKRRRLSSLRASTSKSESSQK |
| SEQ ID NO: 22 | gctctttccggcgggggatctaagctagtctggttgttggccatgaagcttaatatctcgtt |
| NCBI: | cccagccactggctgccaaaagctcattgaagtggaggatgagcgcaagctgcgtact |
| NM_001087120.1 | ttctatgagaagcgcatggccacagaggtcgccgcagatcccttgggtgatgagtgga |
| Xenopus laevis | agggatatgttgttcgcatcagcggtggaaacgataagcaaggcttccccatgaaacag |
| 40S Ribosomal Protein | ggagtcctaactcatggtcgtgttcgtcttctattaagcaagggtcattcctgctatcgccc |
| S6 | caggagaactggtgaacgcaagcgcaaatctgtacgtggatgtattgtggatgctaacc |
| Nucleotide Sequence | tcagtgtcctgaacttggttattgttaggaaaggtgaaaaggatattcctggcctgacaga |
| mRNA | caacactgttcctcgtcgcctgggtcccaaaagagccagcagaatccgcaaactattca |
| | acttgtccaaagaagatgatgtgcgtcagtatgtagtgagaaagcctctggctaaggaa |
| | gggaaaaagcccaggaccaaggcccctaaaatccagcgtctagtgacccccagagtt |
| | ctacagcataagcgcagacgtattgctttgaagaagcagcgtactcaaaagaataagga |
| | agaggcttcagaatatgccaaacttctggctaagagatcaaaggaagccaaggaaaaa |
| | cgccaggagcagatcgcaaagaggcgtagactgtcttctttgagagcctccacatccaa |
| | atctgaatccagtcagaaataaagcttcatcatgtaaaaataaatacattttgttgtaaacaa |
| | aaaaaaaaaaaaaaaaaaaaaaaaaaaa |
| SEQ ID NO: 23 | MKLNISFPATGCQKLIEVDDERKLRIFYEKRMATEVAA |
| NCBI: NP_001003728.1 | DSLGDEWKGYVVRISGGNDKQGFPMKQGVLTHGRVR |
| Danio rerio | LLLSKGHSCYRPRRTGERKRKSVRGCIVDANLSVLNLVI |
| 40S Ribosomal Protein | VRKGEKDIPGLTDSTVPRRLGPKRASRIRKLFNLSKEDD |
| S6 | VRQYVVRRPLTKEGKKPRTKAPKIQRLVTPRVLQHKRR |
| Peptide Sequence | RIALKRQRTLKNKEAAAEYTKLLAKRMKEAKEKRQEQ |
| | IAKRRRLSSLRASTSKSESSQK |
| SEQ ID NO: 24 | ctccaagcgagaaagtcctccatcatgaagctcaatatctcgttccccgccaccggctgc |
| NCBI: | caaaagctgatagaagttgacgatgaacgcaagctgagaatcttctacgagaagcgcat |
| NM_001003728.1 | ggccacagaggtggctgcagactctctgggtgacgagtggaagggctacgttgtgcgc |
| Danio rerio | atcagcggaggcaatgacaaacagggcttccccatgaagcagggtgtgctgacccatg |
| 40S Ribosomal Protein | gacgtgtgcgtctcctcctcagcaagggtcactcttgttaccgtcctcgccgtactggtga |
| S6 | gcgcaaacgcaagtctgtccgcggctgcatcgtcgacgccaacctgagtgttctcaact |
| Nucleotide Sequence | tggtcattgtcaggaagggtgagaaggatattcctgggctgactgatagcactgtccctc |
| mRNA | gccgtctgggacccaagagggctagcaggatccgcaagctcttcaacctgtccaaaga |
| | ggacgatgtcaggcagtatgtggtccggagacctctcactaaagaaggcaagaagccc |
| | aggactaaagcccctaagattcagcgtctggttacaccccgtgtgctgcagcacaagcg |
| | cagacgcatcgctctcaagaggcagcgcacactgaagaacaaggaggcagcagcag |
| | aatacaccaaactgctggccaagaggatgaaggaggccaaggagaaacgtcaagaa |
| | cagattgctaagagacgccgtctttcctctctgagagcctccacatccaagtcagagtca |
| | agccagaagtgagacatgtacctcacaaataaaacatgattttttgaaacattctaaaaaa |
| | aaaaaaaaaaaaaaaaaaa |

### Posttranslationally Modified Ribosomal Proteins

In one aspect, disclosed herein are methods, compositions, and kits for isolating ribosomes and associated mRNA using a reagent that selectively binds to a protein (e.g., a ribosomal protein) comprising a posttranslational modification. In one aspect, the protein is posttranslationally modified in response to a stimulus. In another aspect, a greater percentage of the protein is posttranslationally modified in activated cells. The posttranslational modification can be a transient or reversible modification; for example, the percentage of the protein that is posttranslationally modified can be reduced upon removal of the stimulus. The reduction can occur in a time-frame that is measured in days, hours, minutes, or seconds. The protein can comprise a posttranslational modification at one or more sites; for example, 1, 2, 3, 4, 5 6, 7, 8, 9, 10, 11, 12, 13, 14, 15, 16, 17, 18, 19, 20, or more sites.

A posttranslational modification can be the addition of a hydrophobic group; for example, the posttranslational modification can be myristoylation, palmitoylation, isoprenylation, farnesylation, or geranylgeranylation. A posttranslational modification can be the addition of a chemical group; for example, the posttranslational modification can be acylation, acetylation, formylation, alkylation, methylation, amidation, butyrlation, gamma-carboxylation, glycosylation, malonylation, hydroxylation, iodination, oxidation, phosphorylation, adenylylation, proprionylation, pyroglutamate formation, nitrosylation, succinylation, sulfation, or glycation. A posttranslational modification can be the addition of other proteins or peptides; for example, the posttranslational modification can be SUMOylation, ubiquitination, Neddylation, or Pupylation.

In one aspect, ribosomes and associated mRNA are isolated using a reagent that selectively binds to a protein comprising a posttranslational modification. In one embodiment, the protein can be posttranslationally modified at one or more sites; for example, 1, 2, 3, 4, 5 6, 7, 8, 9, 10, 11, 12, 13, 14, 15, 16, 17, 18, 19, 20, or more sites. In one embodiment, the posttranslational modification is phosphorylation. In one embodiment, the protein is a ribosomal protein. In one embodiment, the protein is a ribosomal protein S6. In one embodiment, the ribosomal protein S6 is phosphorylated at one or more sites; for example, 1, 2, 3, 4, or 5 sites. In one embodiment, the ribosomal protein S6 is phosphorylated at serine 235, serine 236, serine 240, serine 244, serine 247, or a combination thereof. In one embodiment, the ribosomal protein S6 is phosphorylated at serine 244. In one embodiment, the ribosomal protein S6 is a mouse protein.

Isolation of Ribosomes, Polysomes, mRNA

Isolation of Ribosomes

Various methods exist to isolate ribosomes and/or polysomes (ribosomal clusters bound to mRNA), from cells, cultured cells and tissues (see, e.g., Bommer et al., 1997, Isolation and characterization of eukaryotic polysomes, in Subcellular Fractionation, Graham and Rickwood (eds.), IRL Press, Oxford, pp. 280-285; incorporated herein by reference in its entirety). Polysomes can be interchangeably referred to as polyribosomes, ribosomal complexes or ribosomal clusters. In some embodiments, the isolated polysomes (ribosomal-mRNA complexes) contain functional ribosomes, capable of supporting translation, association with mRNA, and/or association with translation factors.

In certain embodiments, the isolation method employed has one or more of the following aspects:
a. Maintenance of ribosomal subunits on mRNA during isolation: translation arresting compounds, such as emetine or cycloheximide can be added to arrest translation, whereby reducing or preventing dissociation of mRNA from the ribosome. In some embodiments, isolation is achieved without crosslinking and crosslinking reagents;
b. Inhibition of endogenous RNAase activity: RNAase inhibitors can be added to buffers to maintain the integrity of the mRNA;
c. Isolation of Polysomes: After tissue or cell homogenization, total polysomes are isolated by preparing a post-mitochondrial supernatant in the presence of at least a high concentration salt buffer, for example about 100-150 mM KCl; and
d. Solubilization of rough ER-bound Polysomes under non-denaturing conditions: Detergent can also be added to release membrane-associated polysomes or ribosomes from endoplasmic reticulum membranes; total polysomes or ribosomes can be collected by centrifugation through, for example, a sucrose cushion.

In other embodiments, variations of the above-described general method are used to isolate membrane-associated polysomes or ribosomes from a total pool of polysomes or ribosomes. This can allow for further enrichment of mRNA encoding secreted or transmembrane proteins. Various methods may be used to isolate membrane-associated polysomes from cultured cells and tissue, e.g., methods that employ differential centrifugation (Hall C, Lim L. Developmental changes in the composition of polyadenylated RNA isolated from free and membrane-bound polyribosomes of the rat forebrain, analyzed by translation in vitro. Biochem J. 1981 Apr. 15; 196(1):327-36), rate-zonal centrifugation (Rademacher and Steele, 1986, Isolation of undegraded free and membrane-bound polysomal mRNA from rat brain, J. Neurochem. 47(3):953-957), isopycnic centrifugation (Mechler, 1987, Isolation of messenger RNA from membrane-bound polysomes, Methods Enzymol. 152: 241-248), and differential extraction (Bommer et al., 1997, Isolation and characterization of eukaryotic polysomes, in Subcellular Fractionation, Graham and Rickwood (eds.), IRL Press, Oxford, pp. 280-285; incorporated herein by reference in its entirety) to isolate the membrane-associated polysomes (Heintz US publication 20050009028 incorporated in its entirety).

Reagents for Use in Isolating Ribosomes/Polysomes

Affinity methods can be used to isolate or purify tagged proteins using methods well known in the art including but not limited to including chromatography, solid phase chromatography precipitation, matrices, immunoprecipitation, co-immunoprecipitation, etc.

In an aspect, a reagent is provided that can selectively bind to a protein in a ribosome or polysome bound to an mRNA. In one embodiment, the reagent selectively binds to the protein whether or not the protein comprises a posttranslational modification. In another embodiment, the reagent selectively binds to the protein comprising a posttranslational modification. In some embodiments, the reagent selectively binds to the protein comprising a posttranslational modification at one or more sites of posttranslational modification. In some embodiments, the reagent has lower or substantially no affinity for the protein that does not comprise a posttranslational modification. The reagent can be an antibody, an aptamer, or other affinity reagent. In one embodiment, the reagent is a polyclonal antibody. In another embodiment, the reagent is a monoclonal antibody. In one embodiment, the protein is phosphorylated ribosomal protein S6 and the reagent is a phospho-S6 240/244 antibody. In another embodiment, the protein is phosphorylated ribosomal protein S6 and the reagent is a phospho-S6 235/236 antibody. In another embodiment, the protein is ribosomal protein S6 and the reagent is an anti-total rpS6 antibody. In another embodiment, the protein is ribosomal protein L26 and the reagent is an anti-rpL26 antibody. In another embodiment, the protein is ribosomal protein L7 and the reagent is an anti-rpL7 antibody.

In some embodiments, the ribosomes are bound to a reagent or affinity reagent that is bound, covalently or non-covalently, to a solid surface, such as a bead, a resin, or a chromatography resin, e.g., agarose, sepharose, and the like. In other embodiments, other methods are used with or in place of affinity purification. In other embodiments, specific polysomes can be isolated utilizing optical sorting, fluorescence-based sorting or magnetic-based sorting methods and devices.

In certain embodiments, polysomes or ribosomes are not isolated from the post-mitochondrial supernatant or even from a cell or tissue lysate before being subject to affinity purification.

### Blocking Peptides/Blocking Reagents

In some embodiments, a reagent that selectively binds to a protein in a ribosome bound to mRNA can bind to the protein at two or more sites. In some embodiments, the two or more sites can comprise a posttranslational modification. In some embodiments, a blocking reagent or blocking peptide is used to decrease a binding affinity of the reagent for one or more sites. In some embodiments, a blocking reagent or a blocking peptide is used to increase the specificity of the reagent for at least one of the two or more sites. The blocking peptide can comprise a posttranslational modification. In one embodiment, the posttranslational modification on the blocking peptide is the same as, or mimics, a posttranslational modification on the protein.

A binding affinity of a reagent for one or more sites on the protein can be between about 1 and 100,000 times lower when a blocking peptide or blocking reagent is used; for example, the affinity can be about 1-100000, 1-50000, 1-10000, 1-5000, 1-1000, 1-500, 1-250, 1-100, 1-10, 10-100000, 10-50000, 10-10000, 10-5000, 10-1000, 10-500, 10-250, 10-100, 100-100000, 100-50000, 100-10000, 100-5000, 100-1000, 100-500, 100-250, 250-100000, 250-50000, 250-10000, 250-5000, 250-1000, 250-500, 500-100000, 500-50000, 500-10000, 500-5000, 500-1000, 1000-100000, 1000-50000, 1000-10000, 1000-5000, 5000-100000, 5000-50000, 5000-10000, 10000-100000, 10000-50000, 50000-100000, 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2750, 3000, 3250, 3500, 3750, 4000, 4250, 4500, 4750, 5000, 5500, 6000, 6500, 7000, 7500, 8000, 8500, 9000, 9500, 10000, 11000, 12000, 13000, 14000, 15000, 16000, 17000, 18000, 19000, 20000, 21000, 22000, 23000, 24000, 25000, 27500, 30000, 32500, 35000, 37500, 40000, 42500, 45000, 47500, 50000, 55000, 60000, 65000, 70000, 75000, 80000, 85000, 90000, 95000, or 100000 times lower when the blocking reagent or peptide is used.

Disclosed herein are methods, compositions, and kits for isolating ribosomes or polysomes and associated mRNA (e.g., actively translated mRNA) using a reagent that selectively binds to phosphorylated ribosomal protein S6. The reagent can be a monoclonal antibody. The reagent can be a polyclonal antibody. In some embodiments, the reagent can bind to two or more sites on the phosphorylated ribosomal protein S6. In one embodiment, the reagent is an anti-pS6 240/244 antibody. In another embodiment, the reagent is an anti-pS6 235/236 antibody. In some embodiments, a blocking peptide is used to decrease an affinity of the reagent for one or more sites on the phosphorylated ribosomal protein S6. In one embodiment, the blocking peptide has a sequence that is about 40%, 50%, 60%, 70%, 80%, 90%, 95%, 99%, or 100% identical to a fragment of a peptide sequence disclosed in Table 2. The fragment can be between about 5 amino acids and about 100 amino acids long; for example, about 5-100, 5-50, 5-25, 5-10, 10-100, 10-50, 10-25, 25-100, 25-50, 50-100, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids long. In another embodiment, the blocking peptide has a sequence that is about 40%, 50%, 60%, 70%, 80%, 90%, 95%, 99%, or 100% identical to SEQ ID NO: 25. The blocking peptide can be phosphorylated on one or more residues.

TABLE 3
Blocking Peptide Sequence

| | |
|---|---|
| SEQ ID | QIAKRRRLpSpSLRApSTSKSESSQK |
| NO: 25 | |

pS = phosphoserine

### Lysate or Fractions of Heterogeneous Cell Populations

Disclosed herein are methods, systems, compositions and kits for isolating mRNA from a lysate or fraction of a heterogeneous population of cells. The heterogeneous population of cells can comprise bacterial or eukaryotic cells. The heterogeneous population of cells can comprise mammalian cells. In one embodiment, the heterogeneous population of cells comprises mouse cells.

A lysate or fraction from which mRNA can be isolated can be derived from any source of cells. In one embodiment, the lysate or fraction is derived from a cell culture. In another embodiment, the lysate or fraction is derived from all or a portion of an organism. In another embodiment, the lysate or fraction is derived from a tissue sample of an organism. In another embodiment, the lysate or fraction is derived from all or a portion of an organ. In another embodiment, the lysate or fraction can be derived from all or a portion of a heart, a salivary gland, an esophagus, a stomach, a liver, a gallbladder, a pancrease, a small intestine, a large intestine, a colon, a rectum, an anus, a hypothalamus, a pituitary gland, a pineal gland, a thyroid, an adrenal gland, a kidney, a bladder, a lymph node, skin, a muscle, a brain, a spinal cord, an ovary, a testicle, a prostate, a penis, a lung, bone marrow, or a combination thereof.

Isolation of mRNA from Ribosomes

Once the ribosome has been isolated, the associated mRNA can be isolated using chemical, mechanical or other methods well known in the art. For example, isolation of mRNA can be accomplished by addition of EDTA to buffers, which can disrupts polysomes and allows isolation of bound mRNA for analysis (Schutz, et al. (1977), Nucl. Acids Res. 4:71-84; Kraus and Rosenberg (1982), Proc. Natl. Acad. Sci. USA 79:4015-4019). In addition, isolated polysomes (attached or detached from isolation matrix) can be directly inputted into RNA isolation procedures using reagents such as Tri-reagent (Sigma) or Triazol (Sigma). In some embodiments, poly A⁺ mRNA is preferentially isolated by virtue of its hybridization of oligo dT cellulose. Methods of mRNA isolation are described, for example, in Sambrook et al., 2001, Molecular Cloning, A Laboratory Manual, Third Edition, Cold Spring Harbor Laboratory Press, N.Y.; and Ausubel et al., 1989, Current Protocols in Molecular Biology, Green Publishing Associates and Wiley Interscience, N.Y., both of which are hereby incorporated by reference in their entireties.

Analyses of mRNA Species

The embodiments described herein provide for translation profiling and molecular phenotyping of a subpopulation of cells in a heterogeneous population of cells. The subpopulation of cells can comprise cells responding to a stimulus (e.g., activated cells). mRNA isolated by any of the methods disclosed herein can be analyzed by any method known in the art. In one aspect, a translational profile of activated cells can be analyzed by isolating the mRNA and constructing cDNA libraries or by labeling the RNA for gene expression analysis, for example by disposing the mRNA on a microarray. Embodiments can utilize techniques described in US2005/0009028, which is herein incorporated in its entirety.

In one aspect, mRNA isolated from activated cells can be used to produce a cDNA library. Such cDNA libraries can be useful for analysis of gene expression modulation in response to a stimuli. The isolated mRNA can also be analyzed using microarrays generated and analyzed by methods well known in the art. Gene expression analysis using microarray technology is well known in the art. Methods for making microarrays are taught, for example, in U.S. Pat. No. 5,700,637 by Southern, U.S. Pat. No. 5,510,270 by Fodor et al. and PCT publication WO 99/35293 by Albrecht et al., which are incorporated by reference in their entireties. By probing a microarray with various populations of mRNAs, transcribed genes in certain cell populations can be identified. Moreover, the pattern of gene expression in cells responding to different stimuli can be readily compared.

The isolated mRNA can be analyzed, for example by northern blot analysis, PCR, RNase protection, etc., for the presence of mRNAs encoding certain protein products and for changes in the presence or levels of these mRNAs depending on manipulation.

Other types of assays may be used to analyze a subpopulation of cells in a heterogeneous population of either in vivo, in explanted or sectioned tissue or in the isolated cells, for example, to monitor the response of the cells to a certain manipulation/treatment or candidate agent (for example, a small molecule, an antibody, a hybrid antibody, an antibody fragment, a siRNA, an antisense RNA, an aptamer, a protein, or a peptide) or to compare the response of the animals, tissue or cells to expression of the target or inhibitor thereof, with animals, tissue or cells from animals not expressing the target or inhibitor thereof. The cells may be monitored, for example, but not by way of limitation, for changes in electrophysiology, physiology (for example, changes in physiological parameters of cells, such as intracellular or extracellular calcium or other ion concentration, change in pH, change in the presence or amount of second messengers, cell morphology, cell viability, indicators of apoptosis, secretion of secreted factors, cell replication, contact inhibition, etc.), morphology, etc.

In some embodiments, the isolated mRNA is used to probe a comprehensive expression library (see, e.g., Serafini et al., U.S. Pat. No. 6,110,711, issued Aug. 29, 2000, which is incorporated by reference herein). The library may be normalized and presented in a high density array, such as a microarray.

In some embodiments, a subpopulation of cells responding to a stimulus can be identified and/or gene expression analyzed using the methods of Serafini et al., WO 99/29877 entitled “Methods for defining cell types,” which is hereby incorporated by reference in its entirety.

Data from such analyses may be used to generate a database of gene expression analysis for different populations of cells in the animal or in particular tissues or anatomical regions, for example, in the brain. Using such a database together with bioinformatics tools, such as hierarchical and non-hierarchical clustering analysis and principal components analysis, cells can be “fingerprinted” for particular indications from healthy and disease-model animals or tissues, co-regulated gene sets for a particular function, and the like.

Some embodiments comprise determining an identity and amount of mRNA isolated from a heterogeneous population of cells wherein a stimulus was applied to a source of the heterogeneous population of cells. Such embodiments can further comprise determining an identity and amount of mRNA isolated from a control sample, wherein a source of the control sample was not exposed to the stimulus or was exposed to a different stimulus. The identity and amount of mRNA can be determined using any means known in the art or disclosed herein.

Some embodiments comprise determining an identity and amount of mRNA isolated from a heterogeneous population of cells using a reagent that selectively binds to a posttranslationally modified protein in a ribosome bound to mRNA. Such embodiments can further comprise determining an identity and amount of mRNA isolated from a total ribosomal fraction of a corresponding heterogeneous population of cells using a reagent that binds to a ribosomal protein regardless of whether the protein comprises a posttranslational modification.

When comparing levels of mRNA isolated from two or more sample, the levels of the mRNA can be normalized to an input level of the mRNA of the same identity in the sample prior to the isolation.

### Applications and Stimuli

The methods, compositions, systems, and kits provided herein can be used to identify mRNA whose translation is modulated in response to a stimulus. The methods, compositions, systems, and kits provided herein can also be used to identify cell types responding to a stimulus. Exemplary stimuli include environmental stimuli, a metabolic or dietary stimuli, application or exposure to a drug or active agent (e.g., a therapeutic agent), or application or exposure to a toxin or carcinogen.

Environmental Stimuli

The methods, compositions, systems, and kits provided herein can be used to identify mRNA whose translation is modulated in response to an environmental stimulus. The methods, compositions, systems, and kits provided herein can also be used to identify cell types responding to an environmental stimulus. Exemplary environmental stimuli include, but are not limited to, elevated or depressed noise levels, elevated or depressed temperatures, and elevated or depressed light levels (e.g., light verses dark; dark rearing animals, etc.).

Metabolic or Dietary Stimuli

The methods, compositions, systems, and kits provided herein can be used to identify mRNA whose translation is modulated in response to a metabolic or dietary stimulus. The methods, compositions, systems, and kits provided herein can also be used to identify cell types responding to a metabolic or dietary stimulus. Exemplary a metabolic or dietary stimuli include, but are not limited to, increased food intake, decreased food intake, vitamin or mineral deficiency, low protein diet, high protein diet, low fat diet, high fat diet, low cholesterol diet, high cholesterol diet, low sugar diet, high sugar diet, low carbohydrate diet, high carbohydrate diet, or feeding during a scheduled time of day or for a scheduled duration.

Application or Exposure to a Drug or Active Agent

The methods, compositions, systems, and kits provided herein can be used to identify mRNA whose translation is modulated in response to a drug or active agent. The methods, compositions, systems, and kits provided herein can also be used to identify cell types responding to a drug or active agent. Exemplary a drugs or active agents include pharmaceutical drugs and illegal narcotics. Exemplary drugs or active agents can also include any drug or active agent used to treat a disease or disorder.

Exemplary pharmaceutical drugs can include, but are not limited to, anaesthetic drugs, antiviral drugs, monoclonal antibodies or other biologics, psychiatric medications (e.g., atypical antipsychotics), chemotherapy drugs, or any other type of drug.

Exemplary anesthetic drugs include, but are not limited to amethocaine, cocaine, lidocaine, prilocaine, bupivacaine, levobupivacaine, ropivacaine, mepivacaine, dibucaine, desflurane, enflurane, halothane, isoflurane, methoxyflurane, nitrous oxide, sevoflurane, xenon, barbiturates (e.g., amobarbital (trade name: Amytal), methohexital (trade name: Brevital), thiamylal (trade name: Surital), thiopental (trade name: Penthothal), etc.), benzodiazepines (e.g., diazepam, lorazepam, midazolam, etc.), etomidate, ketamine, propofol, alfentanil, fentanyl, remifentanil, sufentanil, buprenorphine, butorphanol, diamorphine (diacetyl morphine), hydromorphone, levorphanol, meperidine, methadone, morphine, nalbuphine, oxycodone, oxymorphone, pentazocine, Succinylcholine, decamethonium, mivacurium, rapacuronium, atracurium, cisatracurium, rocuronium, vecuronium, alcuronium, doxacurium, gallamine, metocurine, pancuronium, pipecuronium, and tubocurarine.

Exemplary antiviral drugs include, but are not limited to, abacavir, aciclovir, acyclovir, adefovir, amantadine, amprenavir, ampligen, arbidol, atazanavir, atripla, boceprevir, cidofovir, combivir, darunavir, delavirdine, didanosine, docosanol, edoxudine, efavirenz, emtricitabine, enfuvirtide, entecavir, entry inhibitors, famciclovir, fomivirsen, fosamprenavir, foscarnet, fosfonet, ganciclovir, ibacitabine, imunovir, idoxuridine, imiquimod, indinavir, inosine, interferon type iii, interferon type ii, interferon type i, interferon, lamivudine, lopinavir, loviride, maraviroc, moroxydine, methisazone, nelfinavir, nevirapine, nexavir, nucleoside analogues, oseltamivir (Tamiflu), peginterferon alfa-2a, penciclovir, peramivir, pleconaril, podophyllotoxin, raltegravir, ribavirin, rimantadine, ritonavir, pyramidine, saquinavir, stavudine, tea tree oil, tenofovir, tenofovir disoproxil, tipranavir, trifluridine, trizivir, tromantadine, truvada, valaciclovir (Valtrex), valganciclovir, vicriviroc, vidarabine, viramidine, zalcitabine, zanamivir (Relenza), and zidovudine.

Exemplary monoclonal antibodies or other biologics include, but are not limited to 3F8, 8H9, Abagovomab, Abciximab, Adalimumab, Adecatumumab, Afelimomab, Afutuzumab, Alacizumab pegol, ALD518, Alemtuzumab, Altumomab pentetate, Amatuximab, Anatumomab mafenatox, Anrukinzumab, Apolizumab, Arcitumomab, Aselizumab, Atinumab, Atlizumab, Atorolimumab, Bapineuzumab, Basiliximab, Bavituximab, Bectumomab, Belimumab, Benralizumab, Bertilimumab, Besilesomab, Bevacizumab, Biciromab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, Cedelizumab, Certolizumab pegol, Cetuximab, Citatuzumab bogatox, Cixutumumab, Clazakizumab, Clenoliximab, Clivatuzumab tetraxetan, Conatumumab, Crenezumab, CR6261, Dacetuzumab, Daclizumab, Dalotuzumab, Daratumumab, Denosumab, Detumomab, Dorlimomab aritox, Drozitumab, Ecromeximab, Eculizumab, Edobacomab, Edrecolomab, Efalizumab, Efungumab, Elotuzumab, Elsilimomab, Enavatuzumab, Enlimomab pegol, Enokizumab, Ensituximab, Epitumomab cituxetan, Epratuzumab, Erlizumab, Ertumaxomab, Etaracizumab, Etrolizumab, Exbivirumab, Fanolesomab, Faralimomab, Farletuzumab, FBTA05, Felvizumab, Fezakinumab, Ficlatuzumab, Figitumumab, Flanvotumab, Fontolizumab, Foralumab, Foravirumab, Fresolimumab, Fulranumab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lebrikizumab, Lemalesomab, Lerdelimumab, Lexatumumab, Libivirumab, Lintuzumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nimotuzumab, Nivolumab, Nofetumomab merpentan, Ocrelizumab, Odulimomab, Ofatumumab, Olaratumab, Olokizumab, Omalizumab, Onartuzumab, Oportuzumab monatox, Oregovomab, Otelixizumab, Oxelumab, Ozoralizumab, Pagibaximab, Palivizumab, Panitumumab, Panobacumab, Pascolizumab, Pateclizumab, Patritumab, Pemtumomab, Pertuzumab, Pexelizumab, Pintumomab, Placulumab, Ponezumab, Priliximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Sevirumab, Sibrotuzumab, Sifalimumab, Siltuximab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab, Tigatuzumab, TNX-650, Tocilizumab, Toralizumab, Tositumomab, Tralokinumab, Trastuzumab, TRBS07, Tregalizumab, Tremelimumab, Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab, Vesencumab, Visilizumab, Volociximab, Votumumab, Zalutumumab, Zanolimumab, Ziralimumab, and Zolimomab aritox.

Exemplary psychiatric drugs include, but are not limited to Abilify, Adapin, Adderall, Alepam, Alertec, Aloperidin, Alplax, Alprax, Alprazolam, Alviz, Alzolam, Amantadine, Ambien, Amisulpride, Amitriptyline, Amoxapine, Amfebutamone, Anafranil, Anatensol, Ansial, Ansiced, Antabus, Antabuse, Antideprin, Anxiron, Apo-Alpraz, Apo-Primidone, Apo-Sertral, Aponal, Apozepam, Aripiprazole, Aropax, Artane, Asendin, Asendis, Asentra, Ativan, Atomoxetine, Aurorix, Aventyl, Axoren, Beneficat, Benperidol, Bimaran, Bioperidolo, Biston, Brotopon, Bespar, Bupropion, Buspar, Buspimen, Buspinol, Buspirone, Buspisal, Cabaser, Cabergoline, Calepsin, Calcium carbonate, Calcium carbimide, Calmax, Carbamazepine, Carbatrol, Carbolith, Celexa, Chloraldurat, Chloralhydrat, Chlordiazepoxide, Chlorpromazine, Cibalith-S, Cipralex, Citalopram, Clomipramine, Clonazepam, Clozapine, Clozaril, Concerta, Constan, Convulex, Cylert, Cymbalta, Dapotum, Daquiran, Daytrana, Defanyl, Dalmane, Damixane, Demolox, Depad, Depakene, Depakote, Depixol, Desyrel, Dostinex, dextroamphetamine, Dexedrine, Diazepam, Didrex, Divalproex, Dogmatyl, Dolophine, Droperidol, Desoxyn, Edronax, Efectin, Effexor (Efexor), Eglonyl, Einalon S, Elavil, Elontril, Endep, Epanutin, Epitol, Equetro, Escitalopram, Eskalith, Eskazinyl, Eskazine, Etrafon, Eukystol, Eunerpan, Faverin, Fazaclo, Fevarin, Finlepsin, Fludecate, Flunanthate, Fluoxetine, Fluphenazine, Flurazepam, Fluspirilene, Fluvoxamine, Focalin, Gabapentin, Geodon, Gladem, Glianimon, Guanfacine, Halcion, Halomonth, Haldol, Haloperidol, Halosten, Imap, Imipramine, Imovane, Janimine, Jatroneural, Kalma, Keselan, Klonopin, Lamotrigine, Largactil, Levomepromazine, Levoprome, Leponex, Lexapro, Libotryp Libritabs, Librium, Linton, Liskantin, Lithane, Lithium, Lithizine, Lithobid, Lithonate, Lithotabs, Lorazepam, Loxapac, Loxapine, Loxitane, Ludiomil, Lunesta, Lustral, Luvox, Lyrica, Lyogen, Manegan, Manerix, Maprotiline, Mellaril, Melleretten, Melleril, Melneurin, Melperone, Meresa, Mesoridazine, Metadate, Methamphetamine, Methotrimeprazine, Methylin, Methylphenidate, Minitran, Mirapex, Mirapexine, Moclobemide, Modafinil, Modalina, Modecate, Moditen, Molipaxin, Moxadil, Murelax, Myidone, Mylepsinum, Mysoline, Nardil, Narol, Navane, Nefazodone, Neoperidol, Neurontin, Nipolept, Norebox, Normison, Norpramine, Nortriptyline, Novodorm, Olanzapine, Omca, Oprymea, Orap, Oxazepam, Pamelor, Parnate, Paroxetine, Paxil, Peluces, Pemoline, Pergolide, Permax, Permitil, Perphenazine, Pertofrane, Phenelzine, Phenytoin, Pimozide, Piportil, Pipotiazine, Pragmarel, Pramipexole, Pregabalin, Primidone, Prolift, Prolixin, Promethazine, Prothipendyl, Protriptyline, Provigil, Prozac, Prysoline, Psymion, Quetiapine, Ralozam, Reboxetine, Redeptin, Resimatil, Restoril, Restyl, Rhotrimine, Risperdal, Risperidone, Rispolept, Ritalin, Rivotril, Rubifen, Rozerem, Sediten, Seduxen, Selecten, Serax, Serenace, Serepax, Serenase, Serentil, Seresta, Serlain, Serlift, Seroquel, Seroxat, Sertan, Sertraline, Serzone, Sevinol, Sideril, Sifrol, Sigaperidol, Sinequan, Sinqualone, Sinquan, Sirtal, Solanax, Solian, Solvex, Songar, Stazepin, Stelazine, Stilnox, Stimuloton, Strattera, Sulpiride, Sulpiride Ratiopharm, Sulpiride Neurazpharm, Surmontil, Symbyax, Symmetrel, Tafil, Tavor, Taxagon, Tegretol, Telesmin, Temazepam, Temesta, Temposil, Terfluzine, Thioridazine, Thiothixene, Thombran, Thorazine, Timonil, Tofranil, Tradon, Tramadol, Tramal, Trancin, Tranax, Trankimazin, Tranquinal, Tranylcypromine, Trazalon, Trazodone, Trazonil, Trialodine, Trevilor, Triazolam, Trifluoperazine, Trihexane, Trihexyphenidyl, Trilafon, Trimipramine, Triptil, Trittico, Troxal, Tryptanol, Tryptomer, Ultram, Valium, Valproate, Valproic acid, Valrelease, Vasiprax, Venlafaxine, Vestra, Vigicer, Vivactil, Xanax, Xanor, Xydep, Zamhexal, Zeldox, Zimovane, Zispin, Ziprasidone, Zolarem, Zoldac, Zoloft, Zolpidem, Zonalon, Zopiclone, Zotepine, Zydis, and Zyprexa.

Atypical antipsychotics can include, but are not limited to amisulpride, aripiprazole, asenapine, blonanserin, clotiapine, clozapine, iloperidone, lurasidone, mosapramine, olanzepine, paliperidone, perospirone, quetiapine, remoxipride, risperidone, sertindole, sulpiride, ziprasidone, zotepine, bifeprunox, pimavanserin, and vabicaserin.

### Drugs Used to Treat Diseases and Disorders as Stimuli

The methods, compositions, systems, and kits provided herein can be used to identify mRNA whose translation is modulated in response to a treatment for a disease or disorder. Several disease are cited in, but not limited to those found in the ‘The Merck Manual of Diagnosis and Therapy’, often called simply ‘The Merck Manual’ (2006). Exemplary diseases and disorders can include, but are not limited to, central nervous system disorders, peripheral nervous system disorders, and non nervous system disorders.

Examples of neurodegenerative diseases/disorders include, but are not limited to: alcoholism, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, and Tables dorsalis.

Examples of neuropsychiatric diseases/disorders include, but are not limited to: depression, bipolar disorder, mania, obsessive compulsive disease, addiction, ADHD, schizophrenia, auditory hallucinations, eating disorders, hysteria, autism spectrum disorders and personality disorders.

Examples of neurodevelopmental diseases/disorders include, but are not limited to: attention deficit hyperactivity disorder (ADHD), attention deficit disorder (ADD), schizophrenia, obsessive-compulsive disorder (OCD), mental retardation, autistic spectrum disorders (ASD), cerebral palsy, Fragile-X Syndrome, Downs Syndrome, Rett's Syndrome, Asperger's syndrome, Williams-Beuren Syndrome, childhood disintegrative disorder, articulation disorder, learning disabilities (i.e., reading or arithmetic), dyslexia, expressive language disorder and mixed receptive-expressive language disorder, verbal or performance aptitude. Diseases that can result from aberrant neurodevelopmental processes can also include, but are not limited to bi-polar disorders, anorexia, general depression, seizures, obsessive compulsive disorder (OCD), anxiety, bruixism, Angleman's syndrome, aggression, explosive outburst, self injury, post traumatic stress, conduct disorders, Tourette's disorder, stereotypic movement disorder, mood disorder, sleep apnea, restless legs syndrome, dysomnias, paranoid personality disorder, schizoid personality disorder, schizotypal personality disorder, antisocial personality disorder, borderline personality disorder, histrionic personality disorder, narcissistic personality disorder, avoidant personality disorder, dependent personality disorder, reactive attachment disorder; separation anxiety disorder; oppositional defiant disorder; dyspareunia, pyromania, kleptomania, trichotillomania, gambling, pica, neurotic disorders, alcohol-related disorders, amphetamine-related disorders, cocaine-related disorders, marijuana abuse, opioid-related disorders, phencyclidine abuse, tobacco use disorder, bulimia nervosa, delusional disorder, sexual disorders, phobias, somatization disorder, enuresis, encopresis, disorder of written expression, expressive language disorder, mental retardation, mathematics disorder, transient tic disorder, stuttering, selective mutism, Crohn's disease, ulcerative colitis, bacterial overgrowth syndrome, carbohydrate intolerance, celiac sprue, infection and infestation, intestinal lymphangiectasia, short bowel syndrome, tropical sprue, Whipple's disease, Alzheimer's disease, Parkinson's Disease, ALS, spinal muscular atrophies, and Huntington's Disease. Further examples, discussion, and information on neurodevelopmental disorders can be found, for example, through the Neurodevelopmental Disorders Branch of the National Institute of Mental Health (worldwide website address at nihm.nih.gov/dptr/b2-nd.cfm).

Examples of other diseases or disorders cancers, endocrine diseases, and intestinal diseases.

### Antibodies, Cell Lines, Blocking Peptides, and Kits

In one aspect, provided herein are antibodies or fragments thereof that selectively bind to a protein at one or more sites. In one embodiment, at least one of the one or more sites is posttranslationally modified (e.g., phosphorylated). In another embodiment, each of the one or more sites can be posttranslationally modified. An antibody or a fragment thereof includes, but is not limited to an antibody that comprises one or more light chains and one or more heavy chains, a single-chain antibody, a VHH antibody (variable domain of a heavy chain), a VNAR antibody, or a scFv antibody (a single-chain Fv fragment). An antibody can be an IgA, IgD, IgE, IgG, or an IgM antibody or a fragment thereof. An antibody can be a human, a mouse, a rabbit, a chicken, a donkey, a horse, a camel, or a guinea pig antibody or a fragment thereof. In one embodiment, a single-chain antibody is a single heavy-chain antibody that forms a homodimer. In another embodiment, a single heavy-chain antibody is a camelid antibody. In another embodiment, a single heavy-chain antibody is a camel antibody. In another embodiment, a VHH antibody is a llama antibody. In another embodiment, antibody is a scFv antibody or a fragment thereof. In one embodiment, an antibody or a fragment there of is a human antibody. In another embodiment, an antibody or a fragment there of is a humanized antibody. In another embodiment, an antibody or a fragment thereof can be fused to a polypeptide that is not an antibody or a fragment derived from an antibody.

In some embodiments, an antibody provided herein can selectively bind to a protein at a single site of posttranslational modification. In one embodiment, the antibody is a monoclonal antibody. In one embodiment, the protein is ribosomal protein S6 and the posttranslational modification is phosphorylation. In another embodiment, the protein is ribosomal protein S6 phosphorylated at serine 235, serine 236, serine 240, serine 244, or serine 247. In another embodiment, the protein is ribosomal protein S6 phosphorylated at 244. The antibody can be a monoclonal antibody. In one embodiment, the antibody does not bind, or has substantially lower affinity, for ribosomal protein S6 that is not phosphorylated at serine 244.

Also provided herein are cell lines expressing a monoclonal antibody disclosed herein. The cell line can be a hybridoma. The monoclonal antibody can be an antibody that selectively binds to a ribosomal protein S6 phosphorylated at a single site. The monoclonal antibody can be an antibody that selectively binds to ribosomal protein S6 phosphorylated at serine 235, serine 236, serine 240, serine 244, or serine 247. In one embodiment, the monoclonal antibody selectively binds to ribosomal protein S6 phosphorylated at 244.

In a further aspect, the present invention provides kits. A kit can contain a reagent that selectively binds to a protein in a ribosome bound to mRNA. Such kits can further comprise instructions for use. The reagent can be an antibody, aptamer, or other affinity reagent. The reagent can be a monoclonal antibody. The reagent can be a polyclonal antibody. The reagent can bind to the protein at a site of posttranslational modification. The reagent can bind to the protein at one or more sites. In some embodiments, at least one of the one or more sites comprises a posttranslational modification.

In one aspect, a kit is provided that contains a monoclonal antibody that selectively binds to a ribosomal protein S6 that is phosphorylated at a single site. In one embodiment, the ribosomal protein S6 is phosphorylated at serine 235, serine 236, serine 240, serine 244, or serine 247. In another embodiment, the ribosomal protein S6 is phosphorylated at 244. The kit can further comprise instructions for use.

In another aspect, a kit is provided that contains an antibody that selectively binds to ribosomal protein S6 that is phosphorylated at any of two or more sites. Such kits can further comprise a blocking peptide, such as any of the blocking peptides disclosed herein. Such kits can further comprise instructions for use.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### An Anatomical Map of mTOR Signaling Revealed by Phospho-S6 Capture

The protein kinase mTOR can be a cellular nutrient sensor that can also regulate complex physiology such as aging, energy homeostasis, and diverse functions of the brain. The specificity of mTOR signaling in these contexts can be encoded by the identity of the cells in which the pathway is activated. The results presented herein show that ribosomes containing phosphorylated S6, a marker of mTOR activity, can be immunoprecipitated from homogenates of complex tissues, such as the brain, thereby enriching for the mRNAs selectively translated in cells with active mTOR signaling. This approach was used to identify neurons that activate mTOR in response to light, fasting, leptin deficiency, and osmotic stimulation. It was observed that reticulocytes harbor high levels of pS6, which was traced to iron regulated mTOR signaling during erythrocyte development. As mTOR signaling in the brain can correlate with neuronal activity, this approach provides an unbiased way to identify molecular markers for neurons activated by physiological signals.

Cells can coordinate their rate of growth and proliferation with the availability of nutrients. The serine-threonine kinase mTOR can be one of the proteins responsible for maintaining this balance in eukaryotic cells. mTOR can be activated by conditions that signal energy abundance, such as the availability of amino acids, growth factors, and intracellular ATP. Activated mTOR can phosphorylate downstream targets that promote anabolic processes, such as protein translation and lipid biosynthesis, while suppressing catabolic processes such as autophagy (see, e.g., Zoncu, R., et al. (2011) Nat Rev Mol Cell Biol 12, 21-35, which is hereby incorporated by reference in its entirety).

mTOR can reside in two cellular complexes that have distinct functions and regulation (see, e.g., Loewith et al. (2002) Mol Cell 10, 457-468 and Sarbassov et al. (2004) Curr Biol 14, 1296-1302; each of which is hereby incorporated by reference in its entirety). mTOR complex 1 (mTORC1) can be sensitive to inhibition by the natural product rapamycin and can contain the protein Raptor. Targets of mTORC1 can include S6 kinase (S6K), which can regulate cell size, and the eIF4-E binding protein (4E-BP1), which can regulate cell proliferation through effects on cap-dependent translation (see, e.g., Dowling et al. (2010) Science 328, 1172-1176 and Shima et al. (1998) Embo J 17, 6649-6659; each of which is hereby incorporated by reference in its entirety). mTOR complex 2 (mTORC2) can be resistant to rapamycin and can contain the protein Rictor. mTORC2 can phosphorylate and activate several kinases in the AGC family, such as Akt and SGK, on a sequence known as the hydrophobic motif (see, e.g., Cybulski and Hall (2009) Trends Biochem Sci 34, 620-627; Garcia-Martinez and Alessi (2008) Biochem J 416, 375-385; Sarbassov et al. (2005) Science 307, 1098-1101; each of which is hereby incorporated by reference in its entirety). As Akt itself can activate mTORC1 by phosphorylation of the tuberous sclerosis complex (Tsc), these two kinases can reciprocally regulate each other in response to growth factor signals.

TOR was discovered in yeast, where it can function as a nutrient sensor regulating cell growth and proliferation (see, e.g., Heitman et al. (1991) Science 253, 905-909; which is hereby incorporated by reference in its entirety). This cell-autonomous function can be conserved in higher organisms, and there has been progress in delineating the molecular pathways by which mTOR can control basic cellular processes such as protein translation (see, e.g., Zoncu et al. (2011) Nat Rev Mol Cell Biol 12, 21-35). The process by which mTOR signaling can coordinates the physiology of multicellular organisms such as mammals can be considered to be less understood. Yet recent data show that perturbation of mTOR signaling can have surprisingly specific physiologic effects. An example is the discovery that global inhibition of the mTORC1 pathway, by treatment with rapamycin or deletion of S6K1, can extend the lifespan of mice (see, e.g., Harrison et al. (2009) Nature 460, 392-395 and Selman et al. (2009) Science 326, 140-144; each of which is hereby incorporated by reference in its entirety). mTOR signaling in the brain can also regulate specific neurobiological processes such as the control of food intake, circadian rhythms, learning and memory, and the effects of narcotics and antidepressants (see, e.g., Cao et al. (2010) J Neurosci 30, 6302-6314; Cota et al. (2006) Science 312, 927-930; Li et al. (2010) Science 329, 959-964; Tang et al. (2002) PNAS99, 467-472; each of which is hereby incorporated by reference in its entirety). As the components of the mTOR pathway can be broadly expressed, the effects of mTOR signaling in each of these contexts can be determined by the identity of the cells in which the pathway is activated.

Ribosomal protein S6 was the first target of the mTOR pathway to be identified (see, e.g., Gressner and Wool (1974) J Biol Chem 249, 6917-6925 and Kabat (1970) Biochemistry 9, 4160-4175; each of which is hereby incorporated by reference in its entirety). Activation of mTORC1 can lead to the rapid phosphorylation of S6 by the kinases S6K1 and S6K2 on five C-terminal serine residues (Ser 235, 236, 240, 244, 247). In some settings, the Rsk family can also contribute to phosphorylation at Ser 235/236 (see, e.g., Pende et al. (2004) Mol Cell Biol 24, 3112-3124 and Roux et al. (2007) J Biol Chem 282, 14056-14064; each of which is hereby incorporated by reference in its entirety). Because mTORC1 can activate S6K, treatment with rapamycin can eliminate or reduce phosphorylation at 240/244 and can substantially reduce phosphorylation at Ser 235/236 in nearly every cell that has been tested (see, e.g., Choo and Blenis (2009) Cell Cycle 8, 567-572; which is hereby incorporated by reference in its entirety). Because of this correlation between S6 phosphorylation and mTORC1 activity, pS6 can be used as a marker for active mTORC1 signaling.

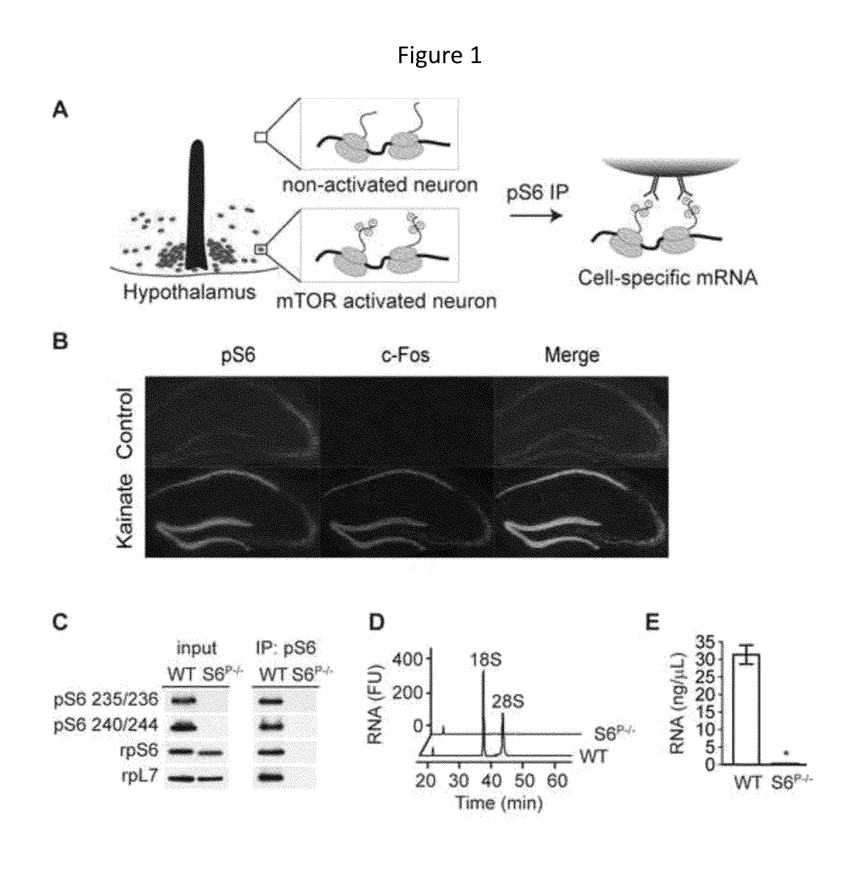

Phosphorylation of S6 introduces a tag on the ribosomes of cells that have active mTORC1 signaling. It was contemplated that it might be possible to use phosphospecific antibodies to selectively immunoprecipitate polysomes comprising pS6 from lysates of complex tissues, such as the brain, thereby enriching for the mRNA derived from the subpopulation of cells with active mTORC1 signaling. An exemplary schematic of this approach is presented in FIG. 1A. By comparing the abundance of each transcript in the pS6 immunoprecipitate to its abundance in the tissue as a whole, it would thus be possible to rank in an unbiased way the genes that were most uniquely expressed in the mTORC1 activated cells. In many cases, these genes would be markers for the specific cell types that underwent mTORC1 activation in response to a physiological stimulus.

A challenge in neuroscience can be to assign functions to the heterogeneous population of neurons in the mammalian brain, which estimates suggest may exceed 1,000 genetically distinct cell types (see, e.g., Masland (2004) Curr Biol 14, R497-500; Nelson et al. (2006) Trends in neurosciences 29, 339-345; and Stevens (1998) Curr Biol 8, R708-710; each of which is incorporated by reference in its entirety). While functional studies can identify anatomical populations of neurons that are co-regulated (e.g., by immunostaining for activation markers), the molecular identification of these cells can be limited because numerous intermingled and morphologically indistinguishable cell types are present in most brain regions. However, emerging evidence indicates that mTORC1 signaling in the brain can be coupled to neuronal activity, as illustrated by the coordinated induction of pS6 and the immediate early gene c-fos in the hippocampus of mice given seizures, as illustrated in FIG. 1B (see also Villanueva et al. (2009) Endocrinology 150, 4541-4551 and Zeng et al. (2009) J Neurosci 29, 6964-6972; each of which is incorporated by reference in its entirety). Because pS6 can be correlated with neuronal activation, the immunoprecipitation of pS6 containing polysomes can represent a way to selectively isolate the mRNA from activated neurons and other cell types, enabling their molecular identification. Described herein is the application of this approach to several classical neurobiological stimuli.

The materials and methods employed in these experiments are now described.

### Materials

The following antibodies were used for immunoprecipitation: rabbit anti-pS6 240/244 (Cell Signaling #2215), rabbit anti-pS6 235/236 (Cell Signaling #4858), rabbit anti-rpL26 (Novus Biologicals, NB100-2131), rabbit anti-rpL7 (Novus Biological, NB100-2269). The following antibodies were used for immunohistochemistry: rabbit anti-pS6 235/236 (Cell Signaling #4858; 40 ng/mL); rabbit anti-pS6 240/244 (Cell Signaling #5364; 50 ng/mL); mouse anti-oxytocin (Millipore, MAB5296; 1:1000), guinea pig anti-vasopressin (Peninsula Laboratories, 1:3000), chicken anti-GFP (Abeam, ab13970; 1:1000), rabbit anti-FosB (Cell Signaling, #2251, 1:25), mouse anti-total rpS6 (Cell Signaling, #2317, 250 ng/mL), rabbit anti-c-fos (Santa Cruz, sc-52, 1/1000), mouse anti-HuC (Invitrogen, 16A11, 1/100), rabbit anti-4EBP1 p37/46 (Cell Signaling, #2855, 1/20). The following additional antibodies were used for western blotting: rabbit anti-hemoglobin (Epitomics, EPR3608, 1:5000), rabbit anti-neuron-specific enolase (Immunostar, #22521, 1/100), HRP-conjugated rabbit anti-actin (Cell Signaling, #5125, 1/2500). The following mice were from Jackson laboratory: POMC-hrGFP (006421), Tsc1^{fl/fl} (005680), NPY-hrGFP (006417), Rosa26-YFP (006148), CNP-eGFP/rpl10a (009159). K562 cells were from ATCC, and the rpS6 mutant and wild-type MEFs were a generous gift.

### Ribosome Immunoprecipitations

Magnetic beads were loaded by incubating 150 μL of Protein A Dynabeads (Invitrogen) with 4 μg of pS6 antibody in Buffer A (10 mM HEPES [pH 7.4], 150 mM KCl, 5 mM MgCl2, 1% NP40, 0.05% IgG-free BSA). Loading was allowed to proceed at 4° C. for a minimum of 1 day. Beads were washed three times with Buffer A immediately before use.

Mice were sacrificed by cervical dislocation. The hypothalamus was rapidly dissected in Buffer B on ice (1×HBSS, 4 mM NaHCO3, 2.5 mM HEPES [pH 7.4], 35 mM Glucose, 100 μg/mL cycloheximide). Hypothalami were pooled (typically 2-5 per IP), transferred to a glass homogenizer (Kimble Kontes 20), and resuspended in 1 mL of buffer C (10 mM HEPES [pH 7.4], 150 mM KCl, 5 mM MgCl2, 100 nM calyculin A, 2 mM DTT, 100 U/mL RNasin, 100 μg/mL cycloheximide, protease and phosphatase inhibitor cocktails). Samples were homogenized three times at 250 rpm and nine times at 750 rpm on a variable-speed homogenizer (Glas-Col) at 4° C. Homogenates were transferred to a microcentrifuge tube and clarified for 10 minutes at 4000 rpm at 4° C. The supernatant was then removed and transferred to a new tube on ice. To this supernatant was added 0.1 volume of 10% NP40 and 0.1 volume of a stock solution of 1,2-diheptanoyl-sn-glycero-3-phosphocholine (DHPC, Avanti Polar Lipids: 100 mg/0.69 mL). This solution was mixed and clarified for 10 minutes at 13000 rpm at 4° C. The supernatant was transferred to a new tube. 50 μL was removed, added to 350 μL buffer RLT (Qiagen), and stored at −80 for purification as input RNA. The remainder was used for immunoprecipitation.

Immunoprecipitations were allowed to proceed for 30-60 min at 4° C. The beads were then washed five times with buffer D (10 mM HEPES [pH 7.4], 350 mM KCl, 5 mM MgCl2, 2 mM DTT, 1% NP40, 100 U/mL RNasin, and 100 μg/mL cycloheximide). The RNA was eluted by addition of 350 μL buffer RLT on ice, the beads removed by magnet, and the RNA purified using the RNeasy Micro Kit (Qiagen). RNA quality was quantified using a NanoDrop spectrophotometer and the quality assessed using an Agilent 2100 bioanalyzer. For microarray analysis, RNA was labeled using the Ovation RNA Amplification System V2 (NuGEN), and hybridized to MouseRef-8 v2 BeadChips (Illumina). For Taqman analysis, cDNA was prepared using the Sensiscript RT kit (Qiagen) and analyzed using an Applied Biosystems 7900HT system.

### Cell Culture Experiments

Wild-type and S6^{S5A} MEFs were cultured in 10% FBS/DMEM/PS. Cells were grown to confluence, starved for 6 hours in 0.25% FBS/DMEM, and restimulated with 20% FBS/DMEM supplemented with 100 nM insulin for 30 minutes. Cells were washed with PBS, trypsinized, collected by centrifugation, and then lysed in a 1% NP40 buffer. K562 cells were grown in RPMI supplemented with 10% dialyzed FBS. Cells were treated for 24 hours with either deferoxamine (30 μM), deferasirox (50 μM), or vehicle (0.05% DMSO), and then collected by centrifugation and lysed in a 1% NP40 buffer.

### Animal Treatment

All animals were 8-12 weeks old at the time of sacrifice. For fasting experiments, animals were transferred to a new cage without food the evening before sacrifice. For dark phase dissections, animals were sacrificed at the midpoint of the dark phase (CT18; 2 am) by cervical dislocation under low power red light. The eyes were also removed under red light, and dissections were then performed as normal. For osmotic stimulation experiments, mice were given an intraperitoneal injection of 2 M NaCl solution (15 μL/g of body weight), transferred to a new cage without water for 2 hours, and then sacrificed.

### Immunohistochemistry

Mice were anesthetized with isoflurane and transcardially perfused with PBS followed by 10% formalin. Brains were dissected, incubated in 10% formalin overnight, and 40 μm sections were prepared on a vibratome. Free floating sections were blocked for 1 hour at room temperature in buffer E (PBS, 0.1% Triton, 2% goat serum, 3% BSA), and then stained overnight at 4° C. Sections were washed with PBS+0.1% Triton (3×20 min); incubated with dye-conjugated secondary antibodies (488, 568, 633) for 1 hour at room temperature; washed in PBS+0.1% Triton (3×20 min), and then mounted.

For immunostaining of PVN/SON neurons, mice were killed by cervical dislocation and brains dissected without perfusion, in order to avoid effects of anesthetics, restraint stress, and perfusion on pS6 levels in these neurons. For double immunostaining of AVP neurons, it was observed that goat anti-rabbit secondary antibodies cross-react with guinea pig primary antibodies; therefore primary antibody incubations were performed sequentially. For 4E-BP1 and FosB staining, primary antibody incubations were allowed to proceed for 72 hours.

### Fluorescent In Situ Hybridization for VIP and NPY

For VIP, a 527 base pair anti-sense digoxigenin-labeled riboprobe was generated from VIP cDNA using a primer set from the Allen Brain Atlas: forward primer CCTGGCATTCCTGATACTCTTC (SEQ ID NO:1)/reverse primer ATTCTCTGATTTCAGCTCTGCC (SEQ ID NO:2). For NPY, a 435 base pair anti-sense digoxigenin-labeled riboprobe was generated from NPY cDNA using the primer set: forward primer TGCTAGGTAACAAGCGAATGG (SEQ ID NO:3)/reverse primer CAACAACAACAAGGGAAATGG (SEQ ID NO:4). 40 μm vibratome free-floating sections were incubated in 3% H₂O₂ for 1 hour at room temperature to quench endogenous peroxidase activity. Sections were treated with 0.20% acetic anhydride followed by 1% Triton-X for 30 min each. Prehybridization was carried out at 37° C. using hybridization buffer (50% formamide, 5×SSC, 5×Denhardts, 250 ng/mL baker's yeast RNA, 500 μg/mL ssDNA) for 1 hour before overnight hybridization with riboprobe at 62° C. Sections were washed in 5×SSC followed by 2 washes with 0.2×SSC at 62° C. Brief washes with 0.2×SSC and buffer B1 (0.1M Tris pH 7.5, 0.15M NaCl) were performed and sections were blocked in TNB (1% blocking reagent in B1, Roche #1096176) for 1 hour at room temperature. Anti-digoxigenin-POD antibody (1:100, Roche #11207733910) was applied overnight at 4° C. Riboprobe was developed using the TSA Plus Fluorescence System (Perkin Elmer, #NEL744) according to the manufacturer's instructions.

### Microscopy and Quantification

Images were acquired using an LSM510 laser scanning confocal microscope. pS6 was quantified in specific neuronal populations as follows. For POMC-hrGFP, AgRP-Cre/Rosa26-YFP and NPY-hrGFP animals, three sections between Bregma −0.94 mm and −1.94 mm were imaged and analyzed for each of three animals from both experimental and control groups. For oxytocin or vasopressin, three sections between Bregma −0.46 mm and −0.82 mm were similarly imaged and analyzed. Z-stack images were acquired and surfaces corresponding to each labeled cell in the field (e.g., each POMC cell) were reconstructed using Imaris software (Bitplane). The mean intensity in the pS6 channel within the volume bounded by the surface of each labeled cell was then recorded and divided into bins to plot pS6 intensity histograms. Images for comparison in this manner were collected using identical microscope and camera settings on tissue samples processed in parallel. All data are presented as mean±SEM and were analyzed by Student's t test.

For comparison of pS6 and total S6 in oligodendrocytes versus neurons, sections from oigodendrocyte reporter mice (CNP-eGFP) were stained for GFP (488), the neuronal marker HuC (568), and either pS6 240/244 or total S6 (633). Z-stack images were acquired and surfaces generated using Imaris to define the oligodendrocytes and neurons in the each slice. Mean intensities for pS6 240/244 and total S6 within each surface were then recorded, as well as the intensity for the marker channels. Cells for which the calculated surface overlapped with markers for both cell-types were excluded (<5% cells); these were defined as cells in which the mean intensity for the primary marker was less than 2.5 fold greater than the mean intensity for the overlapping marker. The mean intensity (signal/volume) for pS6 or total S6 for each individual cell from a field was plotted, and the mean±SEM for all cells in that field was calculated and labeled.

### Hematology

Mice were maintained on either a control diet containing 220 ppm iron (Purina, 5015) or an iron deficient diet containing 2-6 ppm iron (Harlan, TD80396). Mice were additionally given daily subcutaneous injections of deferoxamine (Sigma, 150 mg/kg) in HBSS. Reticulocyte lysates for western blotting were generated by collecting blood in EDTA capillaries by cardiac puncture and diluting into HBSS+20 mM EDTA. This blood was pelleted (3 min at 3000 rpm at 4° C.) and resuspended three times in HBSS/EDTA to remove platelets. The pellet was then resuspended in 0.75 mL of lysis buffer (155 mM NH₄Cl, 10 mM KHCO₃, and 0.01% EDTA) and incubated on ice for 20 min with occasional mixing. This was then centrifuged (5 min at 3000 rpm at 4° C.), and the supernatant collected and used for western blotting. For complete blood counts, blood was diluted 1/10 into HBSS/EDTA and then analyzed using an Advia 120 Hematology analyzer. For perfusion experiments, mice were either killed by cervical dislocation and the hypothalamus dissected directly, or anesthetized with isoflurane and transcardially perfused for 5 minutes with PBS prior to hypothalamic dissection. RNA was prepared from hypothalamic homogenates as described above.

### Analysis of Gene Expression Data

Microarray data was collected for 2-4 independent experiments for each stimulus or control. The ratio of the signal intensities for each gene in the IP (immunoprecipitation) and input was calculated for each experiment, these values were averaged across replicates, and all genes were sorted according to their fold-enrichment. Analysis focused on a small subset of genes corresponding to the most highly enriched or depleted genes in each data set. These were validated independently by Taqman, and in all cases the fold enrichment values were significant at p<0.01 by t-test. The analysis of marker genes for oligodendrocytes, neurons, and astrocytes was based on a previously published set of markers (see Cahoy et al. (2008) J Neurosci 28, 264-278; which is hereby incorporated by reference in its entirety). Mature oligodendrocyte markers were defined as the subset of oligodendrocyte markers that showed greater expression in mature oligodendrocytes than oligodendrocytes as a whole.

### General Considerations for Performing pS6 Immunoprecipitation Experiments

Choice of antibody: multiple commercially available pS6 antibodies were tested and the best results were obtained with a rabbit polyclonal antibody against pS6 240/244 (Cell Signaling, #2211). This antibody was used in all of the experiments reported here except IPs from fasted and ob/ob mice, which used a rabbit monoclonal antibody targeting pS6 235/236 (Cell Signaling #4858). It was found that the latter antibody (#4858) was able to enrich for some mRNAs that are non-specific (e.g., unrelated to pS6). Note that these non-specific mRNAs can be eliminated or substantially eliminated from the analysis by comparing the fold-enrichment (IP/Input) from an experimental group to the fold-enrichment (IP/Input) from controls (see discussion below). Some antibodies were found to be highly non-specific in immunoprecipitation experiments, and therefore not recommended for this application.

Yield versus enrichment: There can be an inverse correlation between the percentage of the input RNA that is recovered in the pS6 IP (yield) and the fold-enrichment (IP/Input) observed for the most highly enriched genes. It was found that the duration of the IP was a factor that can affect RNA yield. In this study, immunoprecipitations were generally performed for 1 hour at 4° C. using the pooled hypothalami from 2-5 mice. Longer incubations can degrade the fold-enrichment; without being bound by theory, this could be explained by non-specific RNA binding to the antibody and magnetic beads. Washing with a solution that has a high-salt concentration can be insufficient to remove non-specific binding.

Immunoprecipitations can be limited to shorter times (e.g., about 5 minutes) in order to enhance the fold-enrichment obtained for the mRNAs associated with the highest density of pS6 ribosomes. This can come at the expense of RNA yield. The optimal balance between yield and enrichment can vary between experiment.

Comparison to controls: The data from these experiments can be analyzed in any suitable fashion. One method of analysis can be to rank each gene according to its fold-enrichment (IP/input) in IPs performed from a tissue prepared under one condition (e.g., the hypothalamus of an unperturbed wild-type mouse). It has been shown that markers for genes in pS6 expressing cells can be identified by this type of analysis (e.g., see the discussion of VIP, oligodendrocyte markers, and hemoglobin supra).

Another method of analysis can include ranking genes according to its fold-enrichment between two or more conditions (e.g., experimental and control conditions). In many cases, the goal of an experiment can be to identify the neurons that are activated by a specific perturbation (e.g., fasting, osmotic stress, drugs, hormones, genetic changes). In these cases, it can simplify the analysis to compare experimental and control groups. In these cases, IP/input values can be calculated separately for an experimental group subjected to the stimulus and a control group that is not. The IP/input for the experimental group can then divided by the IP/Input for the control group, and genes can be ranked according to this ratio. Examples of this type of analysis are given in the discussion on fasting, ob/ob, light-dark, and osmotic stimulation experiments supra.

Because each gene is normalized to its degree of enrichment at baseline, this analysis can enable the identification as enriched of only those genes whose association with pS6 ribosomes changes in response to the specific stimulus. This can simplify the data analysis by eliminates the genes whose enrichment is non-specific (e.g., due to non-specific binding to the antibody or the beads, microarray artifacts, etc.) because these non-specific effects can be observed in both the experimental and control groups. This analysis also takes into account the fact that the association of any transcript with pS6 ribosomes may not be determined exclusively by the amount of pS6 in the cell in which it is expressed, and may be influenced to a varying extent by other factors (e.g., differences in pS6 levels in different subcellular locations, possible differential affinity of messages for pS6 ribosomes, etc.). Comparison to controls can normalize each mRNA individually to its baseline level of association with pS6 ribosomes, and can then ask how that level of association changes in response to the specific stimulus. This can extract the genes whose enrichment is stimulus-specific.

Interpretation of enrichment: The degree of enrichment (IP/input) for a gene in pS6 IPs can be interpreted as measuring the fraction of the mRNA for that gene that is bound to pS6 ribosomes (e.g., the most highly enriched genes can be those for which the highest fraction of their mRNAs are bound to at least one pS6 ribosome). As a result, the highest and lowest fold-enrichment values are generally observed for genes with cell-type restricted expression. Without being limited by theory, this can be because, in a tissue with a heterogeneous pattern of S6 phosphorylation across a field of cells, genes that are expressed in a cell-type restricted way can specifically overlap with (or specifically be excluded from) the subpopulation of cells that have high levels of pS6. Genes that are expressed ubiquitously may not be highly enriched in the subpopulation of cells that have high pS6. Put another way, pS6 immunoprecipitation can enrich for the mRNAs that are most uniquely expressed in the pS6 positive cells, not merely the mRNAs that are most highly expressed in those cells.

The results of the experiments are now described.

### Validation of pS6 Immunoprecipitation

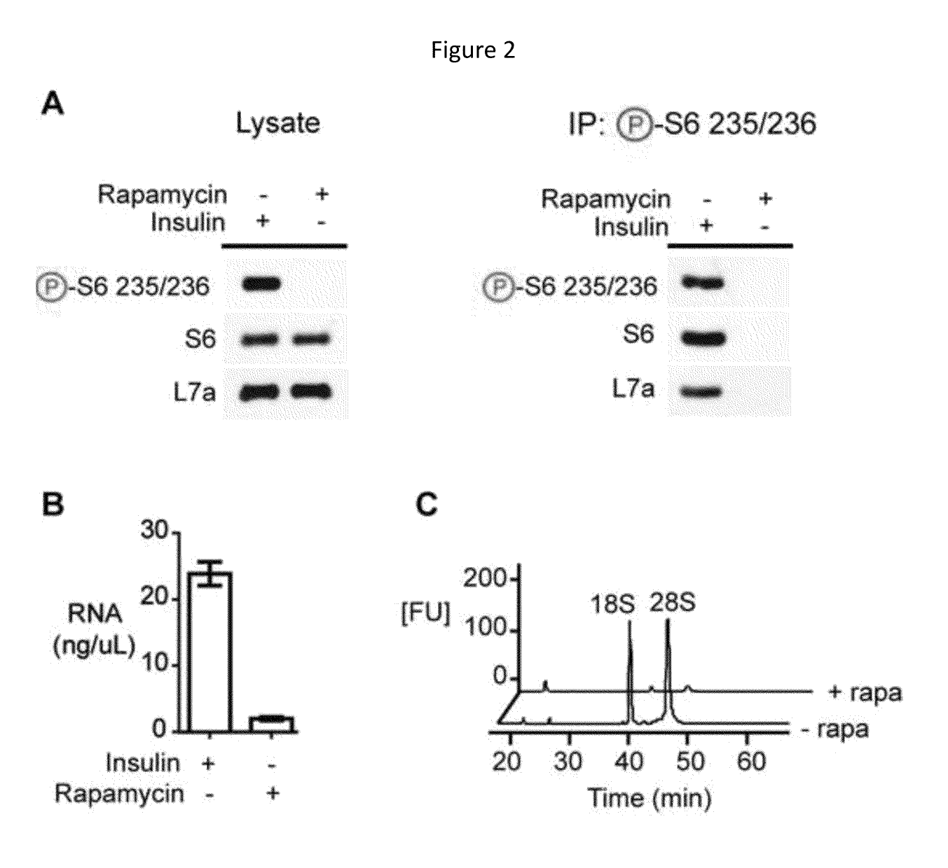

Selective immunoprecipitation of ribosomes containing pS6 was tested in vitro. Mouse embryonic fibroblasts (MEFs) from wild-type mice were compared with MEFs from knock-in mice in which each of the five serine phosphorylation sites on S6 was mutated to alanine (S6S5A) (see, e.g., Ruvinsky et al., (2005) Genes Dev 19, 2199-2211, which is hereby incorporated by reference in its entirety). Serum stimulation induced S6 phosphorylation at Ser 235/236 and Ser 240/244 in wild-type MEFs but not S6S5A mutant cells, as illustrated by the western blots in FIG. 1C, left panel. Lysates were prepared from both cell lines and immunoprecipitations were performed using antibodies against pS6 240/244 Immunoprecipitates from wild-type MEFs but not S6S5A cells recovered ribosomal proteins S6 and L7 as illustrated in FIG. 1C, as well as intact 18S and 28S ribosomal RNA, as illustrated in FIG. 1D. Approximately 100-fold more RNA was associated with immunoprecipitates from wild-type MEFs compared to S6S5A cells, as illustrated in FIG. 1E, confirming that phosphorylated ribosomes and their associated RNA can be selectively isolated. pS6 could also be immunoprecipitated using antibodies against pS6 235/236, as shown in FIG. 2A, right panel. FIG. 2 A-C illustrate that the selective immunoprecipiation of pS6 can be blocked by rapamycin, which can inhibit mTORC1. Immunoprecipitates from NIH3T3 cells that were not treated with rapamycin recovered ribosomal proteins S6 and S7, as shown in FIG. 1A, as well as intact 18S and 28S ribosomal RNA, as shown in FIG. 1C.

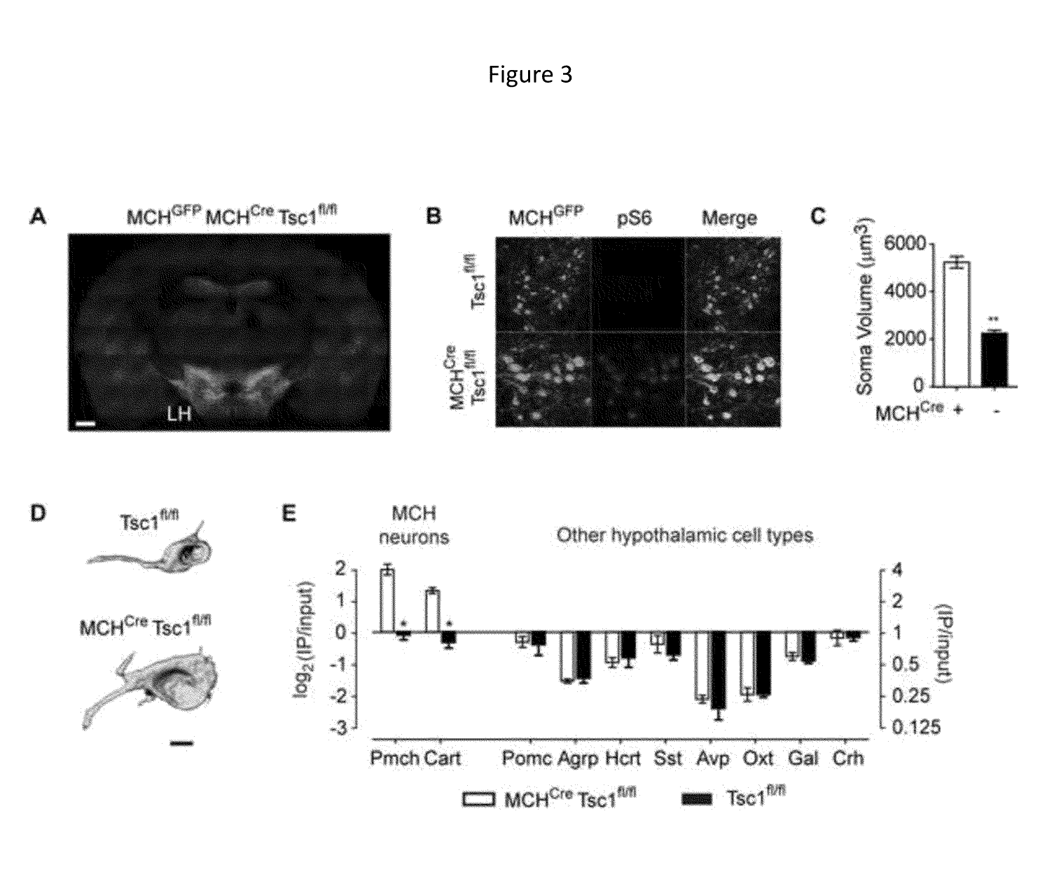

Experiments were designed to confirm that mRNA could be enriched from a single cell type that had mTORC1 activation in vivo. Transgenic mice that express Cre from the melanin concentration hormone (MCH) promoter (MCH^{Cre}) were bred to animals that carry floxed alleles of Tsc1 (Tsc1^{fl/fl}) in order to generate MCH^{Cre} Tsc1^{fl/fl} mice (see Kwiatkowski et al. (2002) Human molecular genetics 11, 525-534; which is hereby incorporated by reference in its entirety). MCH can be expressed in a sparse population of neurons in the lateral hypothalamus that regulate food intake and metabolism, as illustrated by the GFP fluorescence in the brain slice shown in FIG. 3A. In MCH^{Cre} Tsc1^{fl/fl} mice, Tsc1 is selectively deleted from these neurons, which can result in constitutive mTORC1 signaling. To ease visualization of these cells, MCH^{Cre} Tsc1^{fl/fl} mice were additionally bred to an MCH^{GFP} reporter strain (see Stanley et al. (2010) PNAS107, 7024-7029, which is hereby incorporated by reference in its entirety).

Deletion of Tsc1 markedly increased pS6 staining in MCH neurons, as shown in the middle panels of FIG. 3B, and also increased the size of these cells, as quantitated in FIG. 3C and illustrated in FIG. 3D, both of which can indicate active mTORC1 signaling. Tissue homogenates were prepared from whole hypothalami of MCH^{Cre} Tsc1^{fl/fl} mice and immunoprecipitated ribosomes using antibodies against pS6 240/244. Transcripts encoding MCH (Pmch) were enriched in pS6 immunoprecipitates from MCH^{Cre} Tsc1^{fl/fl} mice but not Tsc1^{fl/fl} controls (4.0 versus 0.9-fold, p<0.01; FIG. 3E). Cre dependent enrichment was observed for cocaine and amphetamine related transcript (Cart), a neuropeptide expressed in approximately 45% of mouse MCH neurons (2.5 versus 0.8-fold, p<0.01; FIG. 3E) (see Croizier et al. (2010) PLoS One 5, e15471). By contrast, neuropeptides expressed in a range of other hypothalamic cell types were depleted up to five-fold from pS6 immunoprecipitates, and their degree of enrichment was unaffected by the presence or absence of MCH^{Cre}, as shown in FIG. 3E.

Thus, genetic activation of mTORC1 in a single cell type can enable the enrichment of transcripts unique to that cell in pS6 immunoprecipitates.

Cellular Targets of mTOR Signaling in the Hypothalamus

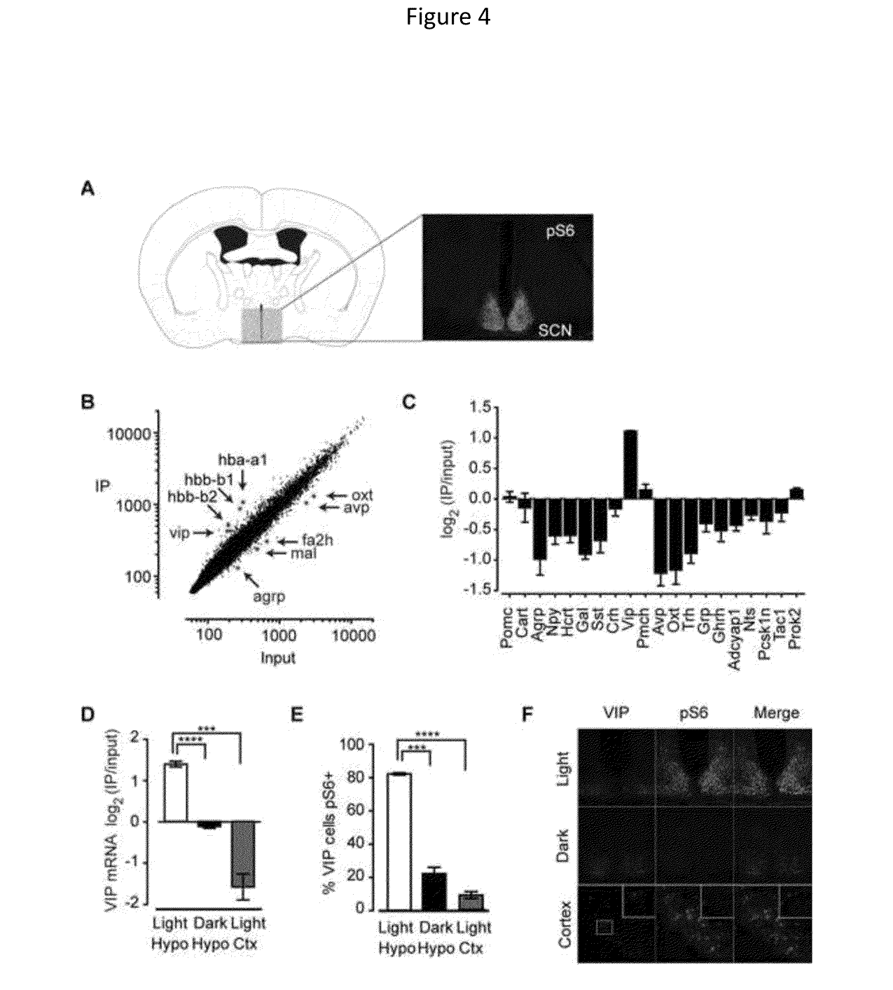

Experiments were performed to profile the cellular targets of mTORC1 signaling in the mouse hypothalamus at baseline. Wild-type mice exhibit strong pS6 immunostaining in the suprachiasmatic nucleus (SCN) during the day, with variable but lower levels of pS6 detectable in other anatomical regions, as illustrated in FIG. 4A. The SCN can control circadian rhythms in response to input from the retina, and light has been shown to activate mTORC1 in a subpopulation of neurons in the SCN (see, e.g., Cao et al. (2008) Mol Cell Neurosci 38, 312-324. and Cao et al. (2010) J Neurosci 30, 6302-6314; each of which is incorporated by reference in its entirety). The neurochemical identity of these cells is unknown.

Wild-type mice were sacrificed near the midpoint of the circadian day (CT 5), prepared tissue homogenates from the hypothalamus, and immunoprecipitated ribosomes using antibodies against pS6 240/244. RNA from pS6 immunoprecipitates (IP) and total hypothalamic RNA (input) were analyzed by microarray. A scatter plot of mRNA abundance for each gene in the pS6 240/244 immunoprecipitate (IP) verses the total hypothalamic RNA (input) is shown in FIG. 4B. Plotted separately in FIG. 4C are the fold-enrichment (IP/Input) for a panel of 20 neuropeptides that represent markers for a series of well-characterized hypothalamic cell types: Ponc, Cart, Agrp, Npy, Hcrt, Gal, Sst, Crh, Vip, Pmch, Avp, Gxt, Trh, Grp, Adcyap1, Nts, Pcsk1n, Tac1, and Prok2.

It was found that three of the top four most enriched transcripts corresponded to the genes for alpha and beta-globin (hba-a1, hbb-b1, hbb-b2; FIG. 4B). The fourth transcript (ccl4) could not detected by Taqman and may be a microarray artifact. Alpha and beta-globin are the polypeptides that comprise hemoglobin, and the origin of these transcripts is discussed infra.

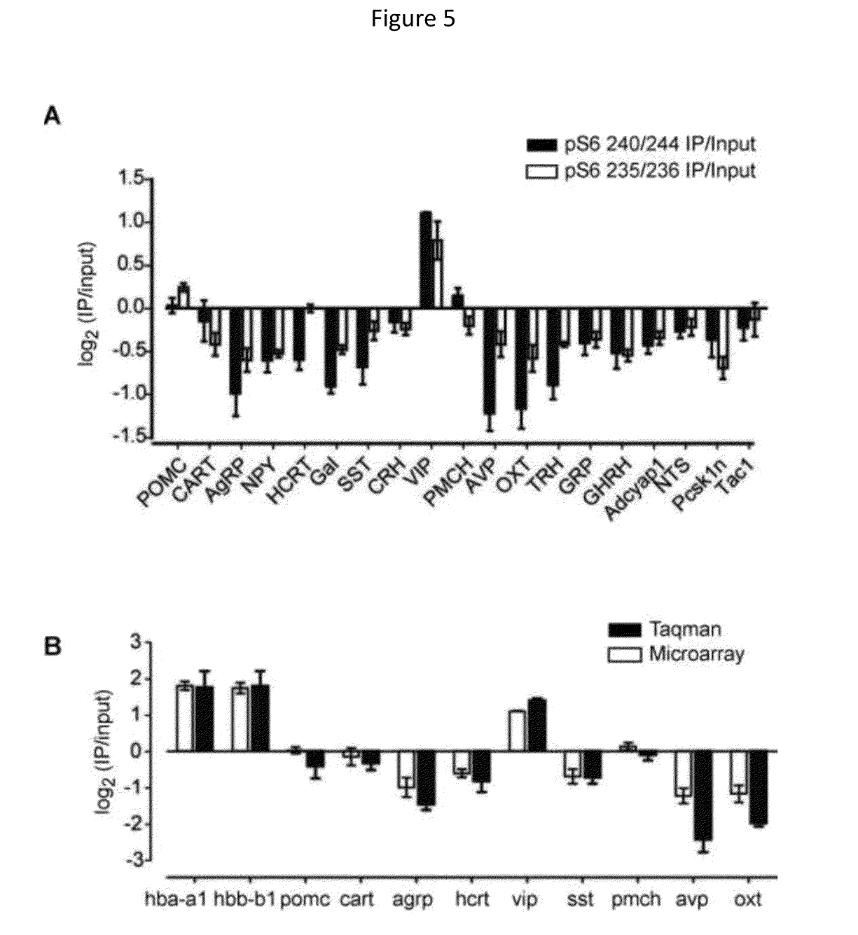

Vasoactive intestinal peptide (VIP) was the only neuropeptide significantly enriched in pS6 immunoprecipitates at baseline (FIG. 4C) and was the 9^{th} most enriched gene overall (2.7 fold by Taqman, p<0.001). As hypothalamic VIP is expressed primarily in the SCN, this suggested that VIP neurons may be the major population of mTORC1 activated cells in that region, and this was confirmed by immunohistochemistry where 82% of VIP cells in the SCN were pS6 positive, as quantitated in FIG. 4E and illustrated by the immunofluorescent images in FIG. 4F. In parallel, immunoprecipitations was performed using antibodies against pS6 235/236, and confirmed that the pattern of enrichment for VIP and other neuropeptides was similar to that observed for pS6 240/244, as shown in FIG. 5A. This is consistent with the idea that phosphorylation at all five sites can be co-regulated (see, e.g., Meyuhas (2008) Int Rev Cell Mol Bio 268, 1-37).

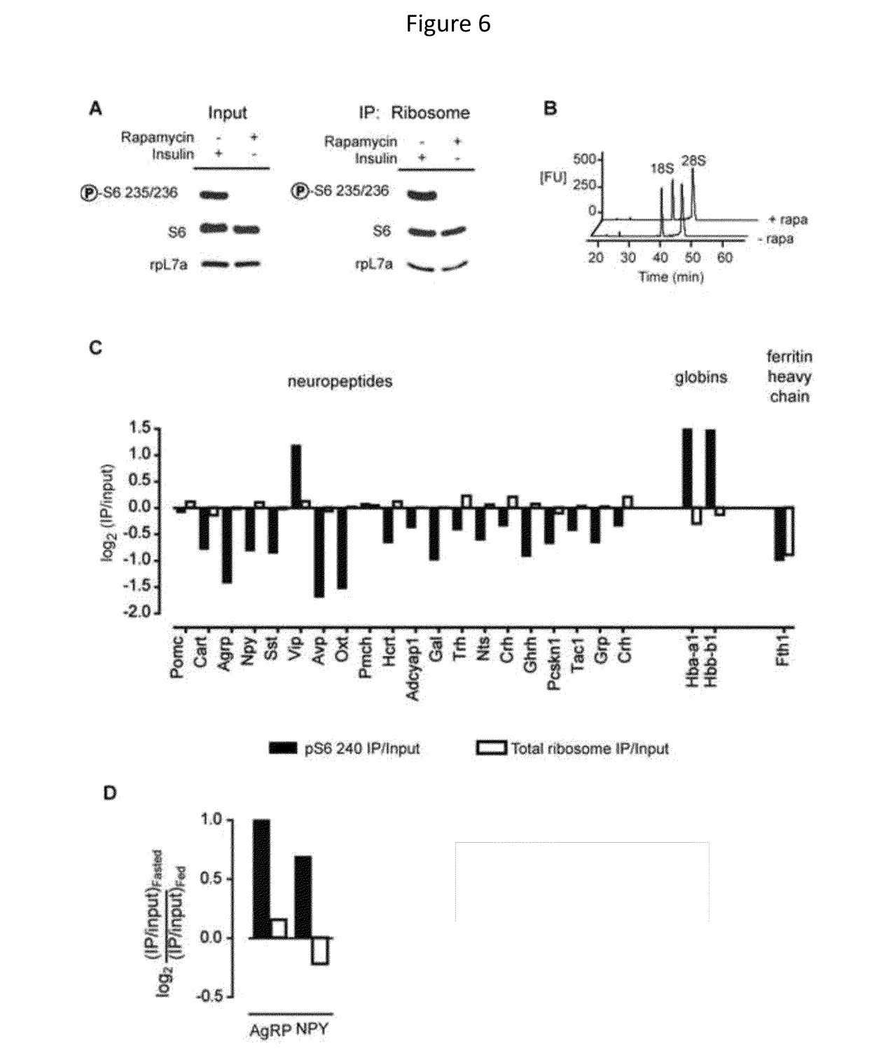

By contrast, immunoprecipitation with a combination of antibodies against ribosomal proteins L7 and L26, which can retrieve all ribosomes, neither enriched nor depleted for the mRNA of any neuropeptide, as illustrated in FIG. 6. In this experiment, NIH3T3 cells were serum starved for 4 h and either restimulated with 20% FBS+100 nM insulin for 30 min or treated with rapamycin for 30 min. Lysates were immunoprecipitated using a combination of antibodies against ribosomal proteins L7 and L26, and the input (FIG. 6A, left) or immunoprecipitate (FIG. 6A, right) was blotted for pS6 235/236 and total ribosomal proteins. The data show that equivalent amounts of ribosomal proteins are recovered in the presence or absense of rapamycin, indicating that the total ribosome immunoprecipitation is not sensitive to the level of pS6. As illustrated in FIG. 6B, Bioanalyzer data of immunoprecipitates show that total ribosome immunoprecipitation recovers similar amounts of RNA in the presence or absence of rapamycin. In another experiment, a hypothalamic homogenate was prepared from a mouse at baseline during the day. One-half of the homogenate was subjected to immunoprecipitation with antibodies against pS6 240/244, and the other half was immunoprecipitated with total ribosome antibodies. Similar amounts of RNA were recovered from the two immunoprecipitates, and this RNA along with the input RNA was analyzed by microarray. The data in FIG. 6C show that immunoprecipitation with pS6 240/244 antibodies (black bars) results in the same pattern of enrichment for neuropeptides and globins as discussed supra. By contrast, immunoprecipitation with total ribosome antibodies (FIG. 6C, white bars) does not show significant enrichment for any of these genes. This indicates that the enrichment can be attributed to mRNA association with pS6 ribosomes, not ribosomes in general. As a positive control, FIG. 6C also shows that both pS6 and total ribosome immunoprecipitation can detect the translational repression of FTH1, a gene that is classically translationally regulated by iron, indicating that both immunoprecipitations can sense the degree of ribosome association. A similar experiment was performed on mice that were either fed or fasted overnight: hypothalamic homogenates were prepared from the mice, half of each homogenate was immunoprecipitated with pS6 240/244 antibodies while the other half was immunoprecipitated with total ribosome antibodies, and the associated mRNA was analyzed by microrarray. FIG. 6D shows a plot of the relative enrichment of AgRP and NPY in pS6 immunprecipitates (black bars) versus total ribosome immunoprecipitates (white bars). These data show that total ribosome immunoprecipitation does not enrich for AgRP and NPY mRNA from fasted mice relative to fed controls.

Thus, the pattern of enrichment observed in these experiments can be consistent mRNA association with pS6 ribosomes, but not ribosomes in general, and may identify VIP neurons as a major pS6 positive cell type in the SCN.

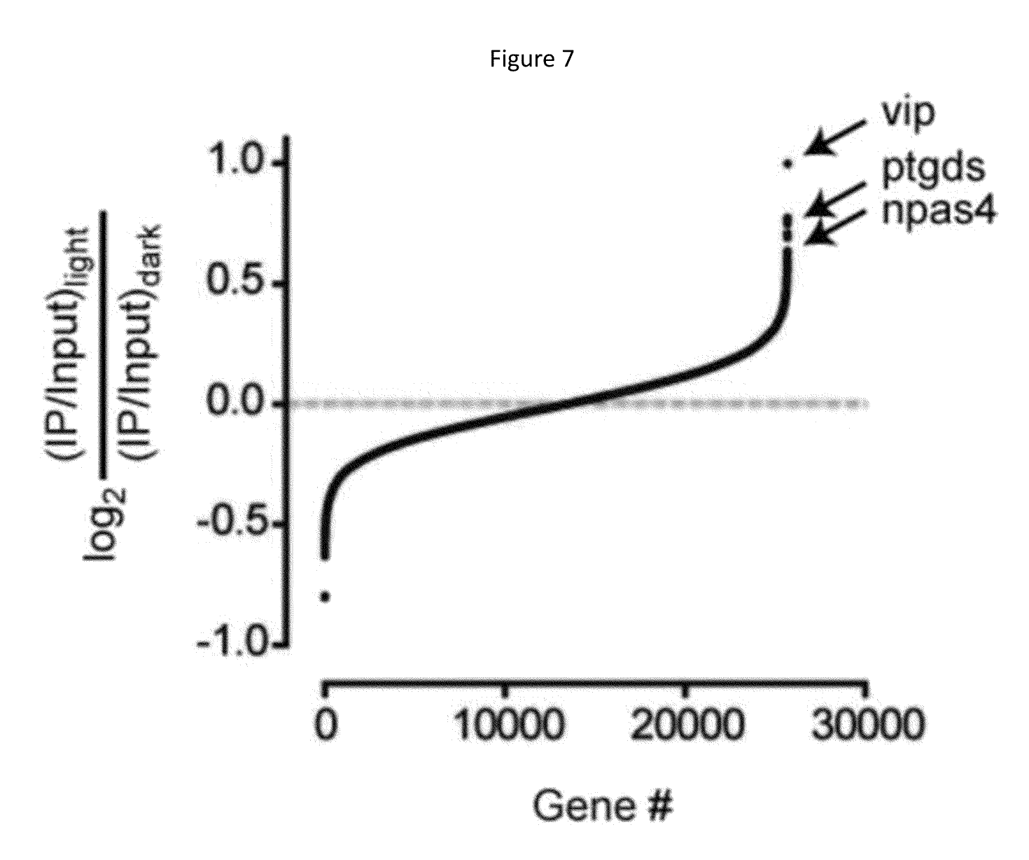

mTORC1 activity in the SCN can be regulated by circadian time and stimulated by light, suggesting that the enrichment observed for VIP could be sensitive to the time of day that the experiment is performed. Mice were sacrificed in the dark at the midpoint of the circadian night (CT 18) and analyzed the RNA recovered in pS6 immunoprecipitates. Night-time dissection abolished the enrichment for VIP mRNA in pS6 immunoprecipitates, as shown in FIG. 4D, and it was confirmed by immunohistochemistry that mice sacrificed in the dark had significantly fewer pS6 positive VIP neurons in the SCN (see FIG. 4E, F). Microarray analysis revealed that VIP was the single most differentially enriched gene detected in pS6 immunoprecipitates from the day versus the night (see FIG. 7), consistent with the idea that this neuropeptide can mark the mTORC1 activated cells in the SCN.

VIP can also be expressed in the cortex, where it can define a major class of interneurons that may be functionally unrelated to VIP neurons of the SCN. Little co-localization was observed between pS6 and VIP neurons in the cortex by immunostaining (FIG. 4E, F) and, consistent with this, it was found that VIP mRNA was markedly depleted in pS6 immunprecipates from this region (FIG. 4D). Thus, these data show that pS6 capture can reveal cell-type specific changes in mTORC1 activity across circadian time and anatomical space.

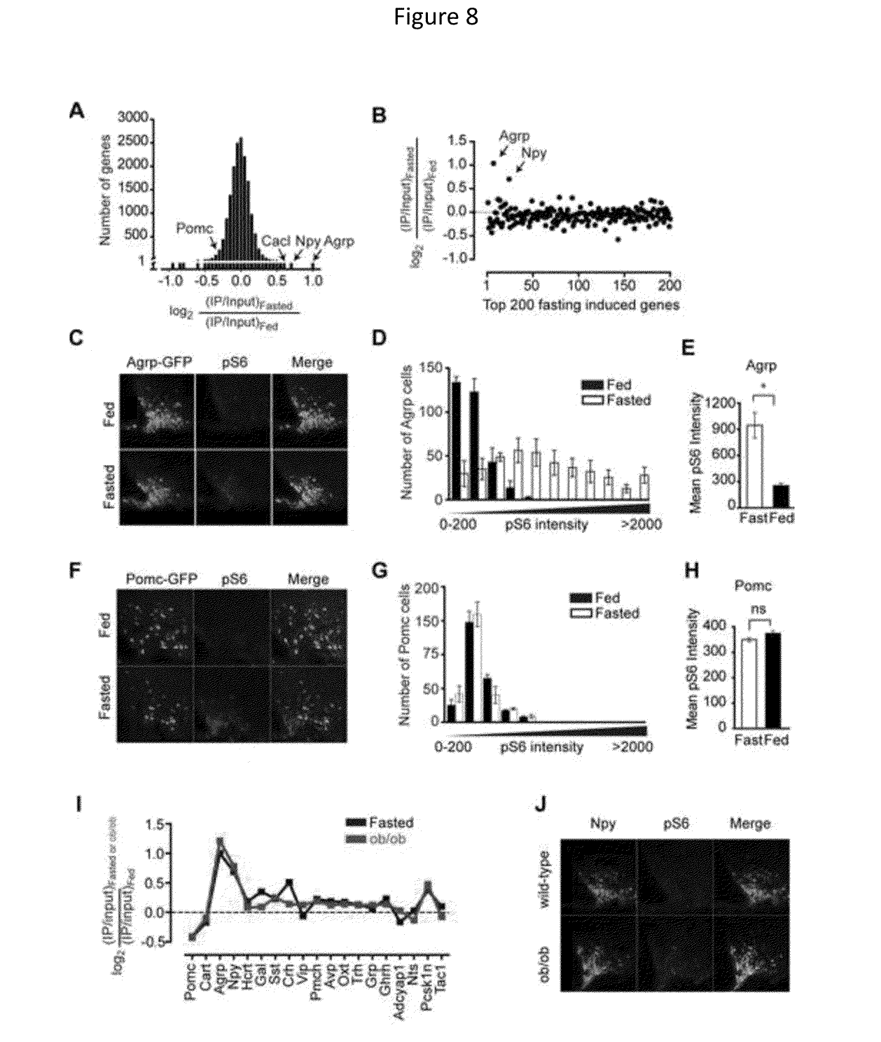

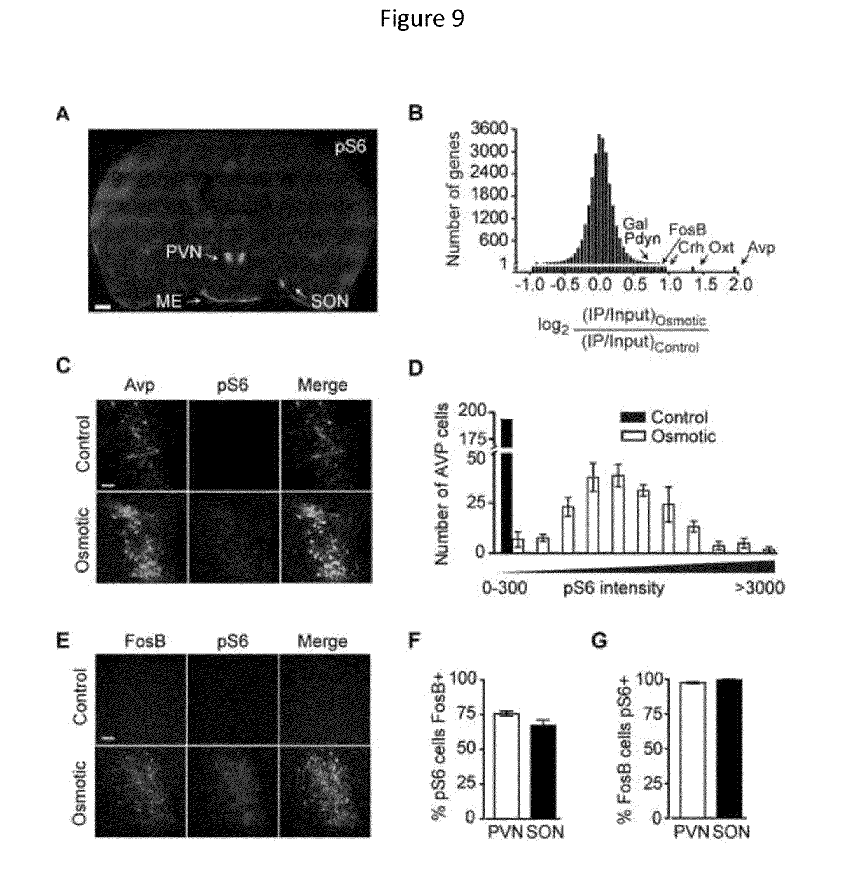

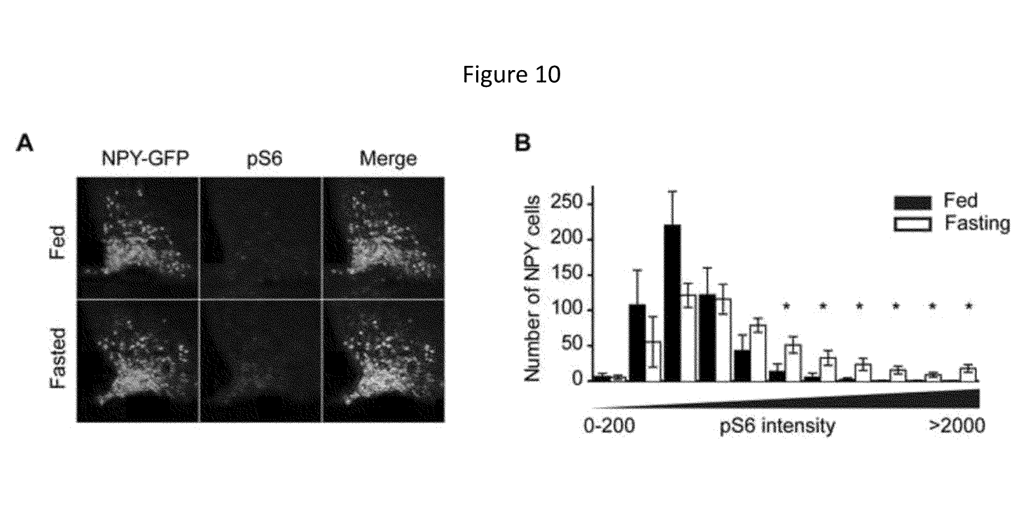

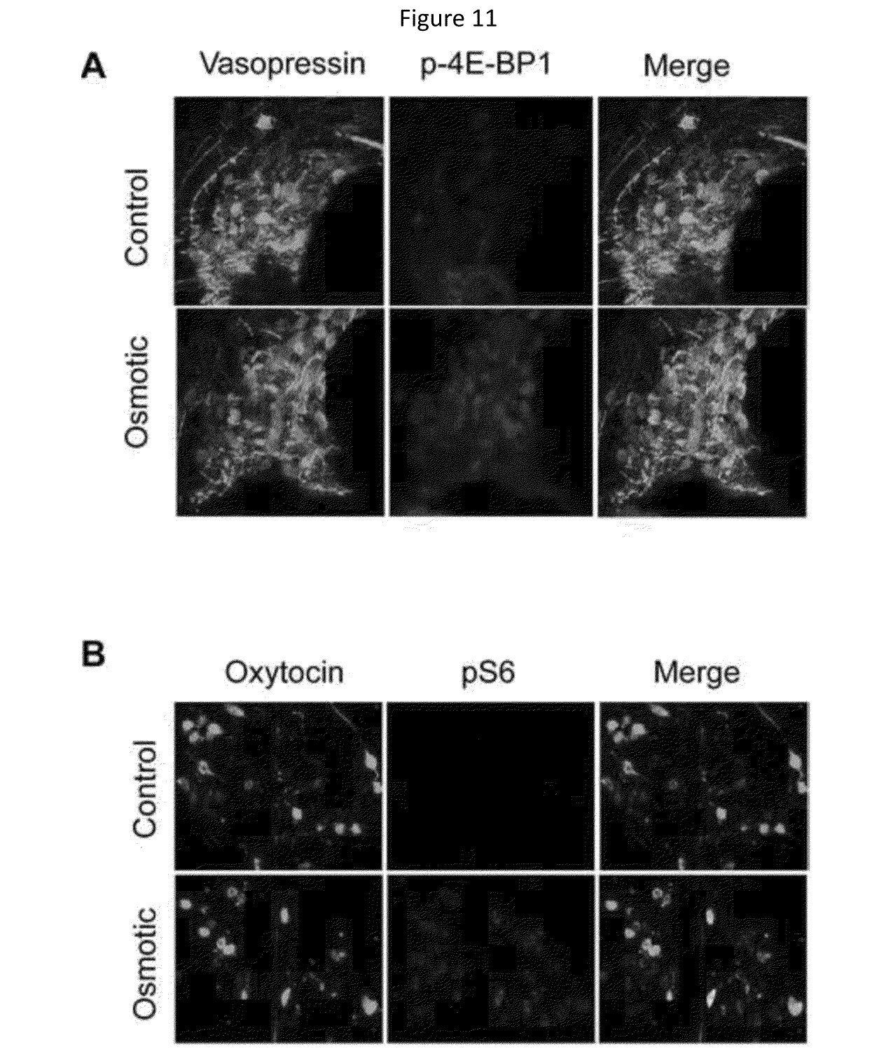

The relative enrichment of marker genes in pS6 immunoprecipitates can reveal the landscape of mTORC1 activity across the numerous cell-types of the hypothalamus at baseline. For this reason, the most depleted transcripts in pS6 immunoprecipitates can provide information about the cells with the lowest basal mTORC1 activity, and numerous markers for well-characterized hypothalamic neurons were found among these genes. For example, five neuropeptides were among the 15 most depleted genes from pS6 immunoprecipitates: galanin (gal), thyrotropin releasing hormone (trh), vasopressin (avp), oxytocin (oxt), and agouti-related protein (agrp). Each of these neuropeptides can be expressed in an anatomically and functionally defined population of hypothalamic neurons, and it has been confirmed in several cases that these neurons have low basal mTORC1 signaling (see, e.g., FIG. 8, FIG. 9, FIG. 10 and FIG. 11).

Activation of Hypothalamic mTORC1 by Metabolic Signals

Experiments were performed to profile how the cellular targets of mTORC1 in the hypothalamus change in response to a set of acute stimuli, focusing first on metabolic signals. Previous work has shown that fasting can induce pS6 in Agrp/Npy neurons (see, e.g., Villanueva et al. (2009) Endocrinology 150, 4541-4551, which is hereby incorporated by reference in its entiretly), a population of cells in the arcuate nucleus that can promote food intake (see, e.g., Aponte et al. (2011) Nat Neurosci 14, 351-355; which is hereby incorporated by reference in its entirety). As Agrp and Npy were identified based on their functional role in feeding, genes marking other hypothalamic cell types might also show mTORC1 activation in response to fasting. Mice were therefore fasted overnight, immunoprecipitated pS6 ribosomes from hypothalamic tissue homogenates, and analyzed the purified RNA by microarray.

To identify the genes that become enriched in pS6 immunoprecipitates specifically as a result of fasting, the ˜25000 transcripts were ranked on the array according to the ratio of their fold enrichment in fasted mice versus their fold enrichment in fed controls. This analysis revealed that the two most differentially enriched genes in pS6 immunoprecipitates were Agrp and Npy (FIG. 8A). This suggests that these two neuropeptides may in fact represent the most uniquely expressed genes in the hypothalamic neurons that activate mTORC1 during fasting. The third most enriched gene was Slc25a29 (also known as CACL), a mitochondrial acylcarnitine transporter that is known to be regulated by fasting and preferentially expressed in the brain (see, e.g., Sekoguchi et al. (2003) J Biol Chem 278, 38796-38802, which is hereby incorporated by reference in its entirety). CACL transports fatty acids into the mitochondria so that they can undergo oxidation, and the substrate for CACL is palmitoylcarnitine, which is generated by the enzyme carnitine palmitoyltransferase (CPT). CPT isoforms, fatty acid metabolism, and mTOR signaling have been linked to the hypothalamic control of food intake (see, e.g., Wolfgang and Lane (2011) The FEBS journal 278, 552-558, which is hereby incorporated by reference it its entirety).

Control experiments were performed in which total ribosomes were immunoprecipitated from fasted mice (FIG. 6) and confirmed that the enrichment for Agrp and Npy was specific to pS6 ribosomes, not ribosomes in general. It was also noted that, although Agrp and Npy expression increases overall during fasting, this increase was not the cause of the enrichment observed for these genes. This is because enrichment was calculated as the ratio of RNA abundance in the IP divided by the input. To show this a different way, 200 transcripts whose overall expression increased to the greatest degree in response to fasting in the hypothalamus was examined There was no trend toward enrichment of these genes in pS6 immunoprecipitates (see FIG. 8B).

Quantitative immunohistochemistry was ised to confirm that fasting increased the density of pS6 in neurons that express AgRP (FIG. 8C-E). This was independently confirmed by immunohistochemistry in neurons that express NPY-GFP (FIG. 10). As control, the amount of pS6 was quantified in Pomc neurons, a cell type that is intermingled with AgRP neurons in the arcuate nucleus but which is not activated by fasting. Consistent with the profiling data, there was no increase in the amount of pS6 in Pomc neurons in response to fasting, even though there was an overall increase in the amount of pS6 in the surrounding cells of the arcuate nucleus (FIG. 8F-H). Thus, the data suggest that Agrp/Npy neurons are a population of cells in the hypothalamus that activate mTORC1 in response to fasting.

Plasma Hyperosmolarity can Activate mTORC1 in the Hypothalamus

It was contemplated whether other physiological signals might activate mTORC1 in specific hypothalamic neurons. One system that can be regulated by the hypothalamus is plasma osmolarity. In response to increases in the salt concentration of the blood, neurons in the paraventricular (PVN) and supraoptic (SON) nuclei can become activated and release neuropeptides, such as vasopressin, that can prevent fluid loss from the kidney. To test whether mTORC1 is activated by changes in plasma osmolarity, mice were injected with a concentrated salt solution and then characterized the effects on hypothalamic mTORC1 signaling.

Osmotic stimulation can induce immunostaining for pS6 in the PVN, SON, and internal layer of the median eminence (ME) of the hypothalamus (FIG. 9A). Osmotic stimulation can also induce the phosphorylation of 4E-BP1 (T37/46), a direct target of mTORC1 kinase activity (FIG. 10A). Thus, increases in plasma osmolarity can activate mTORC1 signaling in a subpopulation of hypothalamic neurons.

To identify the cell types that can activate mTORC1 in response to osmotic stimulation, mice were challenged with a salt injection, immunoprecipitated pS6 polysomes from hypothalamic tissue homogenates, and characterized the transcripts enriched in immunoprecipitates relative to controls. The four most enriched genes were vasopressin (Avp), oxytocin (Oxt), corticotropin releasing hormone (Crh), and FosB (FIG. 9B). Avp and Oxt encode neuropeptides that can be expressed in two populations of neurons in the PVN and SON that can be regulated by plasma osmolarity (see, e.g., Pirnik and Kiss (2005) Brain Res Bull 65, 423-431 and Pirnik et al. (2004) Neurochem Int 45, 597-607; each of which is hereby incorporated by reference in its entirety), whereas Crh can be expressed in a subpopulation of PVN neurons that can partially overlap with both Avp and Oxt (see, e.g., Sawchenko et al. (1984a) PNAS81, 1883-1887 and Sawchenko et al. (1984b) J Neurosci 4, 1118-1129; each of which is hereby incorporated by reference in its entirety). FosB is a transcription factor related to the immediate early gene c-fos. FosB transcription can be directly regulated by neuronal activity (see, e.g., McClung et al. (2004) Mol Brain Res 132, 146-154; which is hereby incorporated by reference in its entirety). Thus, ranking the genes enriched in pS6 immunoprecipitates can reveal the molecular identity of the cell-types activated by osmotic stimulation. Enrichment was also observed, at a lower level, for genes whose expression can partially overlap with these three cell types. For example, the fourth and fifth most enriched neuropeptides were galanin (Gal) and dynorphin (Pdyn), which can be expressed in a subset of oxytocin and vasopressin neurons (FIG. 9B) (see, e.g., Meister et al. (1990) Neuroscience 37, 603-633 and Melander et al. (1986) J Neurosci 6, 3640-3654; each of which is hereby incorporated by reference in its entirety).

It was confirmed by Taqman that the top ranked gene, Avp, was enriched 7.9-fold in pS6 IPs from salt challenged animals relative to controls (p<0.001), and it was validated by immunohistochemistry that osmotic stimulation induced robust pS6 in vasopressin neurons (FIG. 9C). Phosphorylation of 4E-BP1 was also co-localized with this cell population (FIG. 11A). It further confirmed that pS6 was induced in oxytocin neurons; for this cell population, there was pronounced overlap between oxytocin and pS6 staining in the ventral PVN and SON but not the dorsal PVN (FIG. 11B).

As FosB and pS6 represent two different types of markers for neuronal activation—one transcriptional and one post-translational—their degree of co-localization was examined, as quantified in FIG. 9D. Essentially every cell that expressed FosB in the PVN and SON was also pS6 positive (see FIG. 9E), whereas the majority (˜70%) of the pS6 positive cells expressed FosB. Thus, pS6 was detected in a somewhat broader population of cells than FosB. Without wishing to be bound by any particular theory, it is suspected that FosB may fall below the threshold for immunohistochemical detection in some cells that nonetheless were identified as activated by pS6 staining, as a result of the fact that FosB protein expression involves both transcription and translation. Thus it was found that in response to three stimuli—light, fasting and hyperosmolarity—it is possible to identify molecular markers for the neurons that activate mTORC1 signaling by characterizing the transcripts enriched in pS6 polysomes.

Mature Oligodendrocytes have Low mTORC1 Signaling

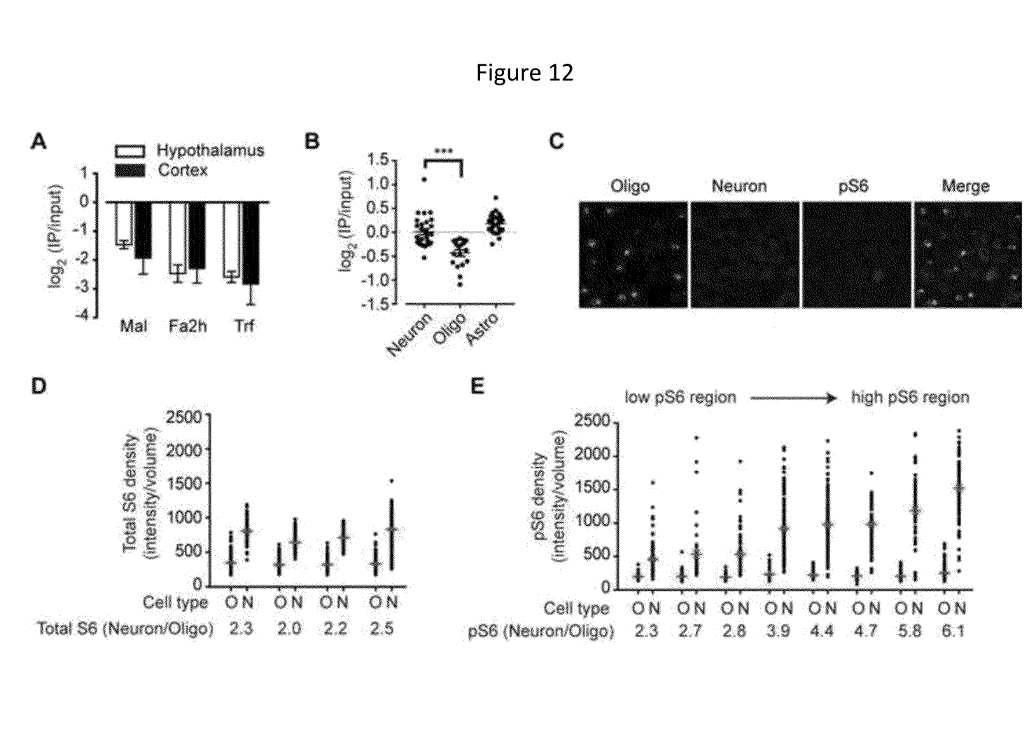

It was noticed in the initial profiling of the hypothalamus at baseline that numerous oligodendrocyte-specific genes were among the transcripts most depleted from pS6 immunoprecipitates (FIG. 4B). Oligodendrocytes are the cells responsible for synthesizing the myelin sheath that surrounds and insulates axons. Myelin synthesis occurs during early postnatal life and is completed by adulthood. For this reason, oligodendrocytes from adult mice may be translationally quiescent relative to other cell types, and therefore have a lower demand for mTORC1 signaling. It was confirmed by Taqman that the oligodendrocyte markers myelin and lymphocyte protein (mal), fatty acid 2-hydroxylase (fa2h), and transferrin (trf) were depleted by 3 to 6-fold in pS6 immunoprecipitates from both the hypothalamus and the cortex at baseline (FIG. 12A). To address this systematically, the degree of enrichment in pS6 immunoprecipitates for a panel of marker genes that can be selectively expressed in neurons, astrocytes, or mature oligodendrocytes (see, e.g., Cahoy et al. (2008) J Neurosci 28, 264-278, which is hereby incorporated by reference in its entirety) is plotted in FIG. 12B. This confirmed that markers for mature oligodendrocytes, but not neurons or astrocytes, are depleted from pS6 IPs (p<0.001), indicating that these cells have low basal mTORC1 activity.

To confirm this immunohistochemically, triple-labeling was performed for oligodendrocytes, neurons, and either pS6 240/244 (FIG. 12C; “pS6”) or total S6 (not shown) in brain sections from mice and then quantified by confocal imaging the density of pS6 and total S6 in these two cell types (FIG. 12 D,E). It was found that, in brain regions with low levels of pS6, the stoichiometry of S6 phosphorylation was slightly higher in neurons than in oligodendrocytes. By contrast, in brain regions with high pS6 staining, the pS6 stoichiometry in neurons was much higher (FIG. 12 E). In FIG. 12E, 8 anatomic fields that are representative of low and high pS6 regions are quantified. Note that the fields are ordered according to increasing pS6 signal, because the intensity of pS6 staining is more variable across brain regions than total S6. The data show that this increasing pS6 signal is concentrated in the neurons but not the oligodendrocytes.

These data suggest a model in which basal mTORC1 activity in adult oligodendrocytes is generally low, whereas mTORC1 activity in neurons is higher and varies according to their activation status. This model is consistent with functional data indicating that oligodendrocytes require mTORC1 activity during development but not adulthood (see, e.g., Narayanan et al. (2009) J Neurosci 29, 6860-6870; which is hereby incorporated by reference in its entirety).

Reticulocytes have Iron-Dependent mTORC1 Signaling

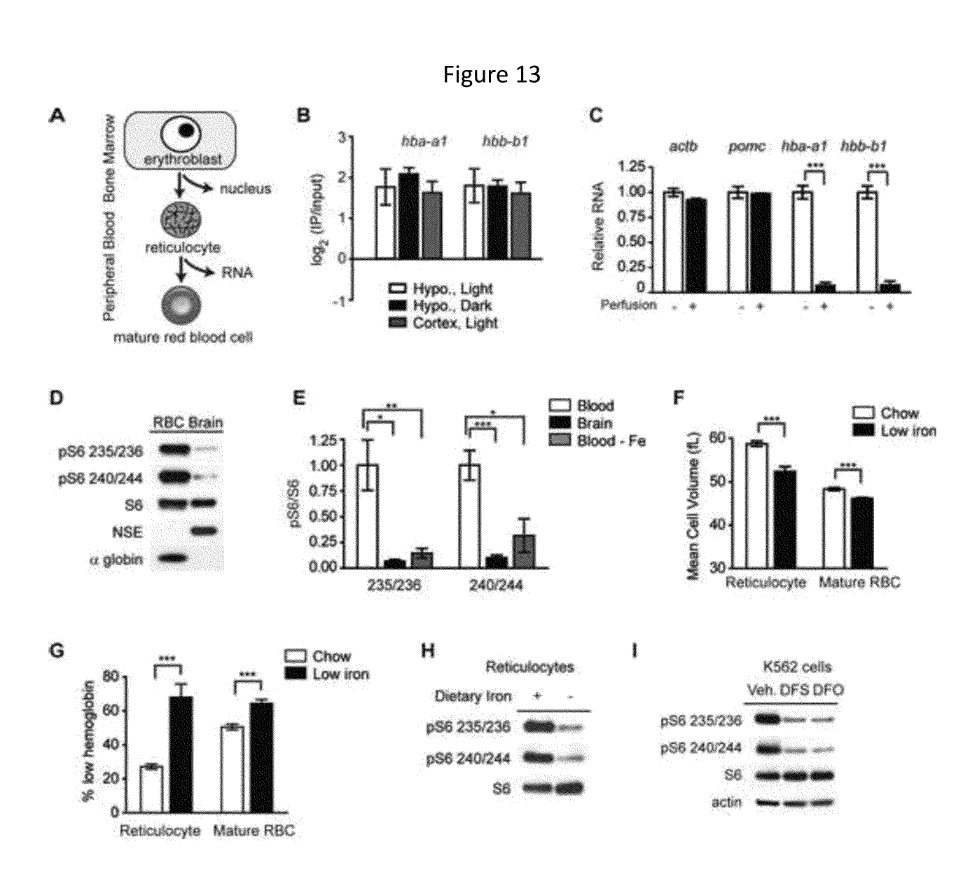

One objective was to identify neurons with active mTORC1 signaling by immunoprecipitation of pS6 polysomes. The finding that the genes for alpha and beta globin (hba-a1, hbb-b1 and hbb-b2) represented three of the top four transcripts enriched in pS6 immunoprecipitates from the hypothalamus was puzzling. It was confirmed by Taqman that hba-a1 and hbb-b1 were enriched in pS6 immunoprecipitates from both the hypothalamus and cortex and that their degree of enrichment was unaffected by circadian time (FIG. 13B), suggesting that these transcripts do not originate in the pS6 positive neurons in the SCN or even in a specific hypothalamic cell type. Despite recent reports indicating that hemoglobin is expressed in the brain (see, e.g., Biagioli et al. (2009) PNAS106, 15454-15459 and Richter et al. (2009) J Comp Neurol 515, 538-547; each of which is hereby incorporated by reference in its entirety), it was not possible to detect reproducible staining for alpha-globin in hypothalamic neurons or glia using multiple commercial antibodies. The possibility that the globin transcripts originated from some other cell population with active mTORC1 signaling was therefore considered.

FIG. 13A is a simplified schematic of red blood cell development. Alpha and beta globin assemble as a tetramer to form hemoglobin, which is produced primarily by red blood cells (RBC). About 75% of hemoglobin is synthesized by RBC progenitors that reside in the bone marrow. As these cells mature, they are released from the bone marrow and extrude their nucleus, becoming reticulocytes that circulate in the peripheral blood for up to a week. Circulating reticulocytes synthesize the remaining ˜25% of RBC hemoglobin, and transcripts for alpha and beta globin can account for the vast majority of the mRNA in these cells (see, e.g., Bonafoux et al. (2004) Haematologica 89, 1434-1438; which is hereby incorporated by reference in its entirety). As reticulocytes gradually become mature erythrocytes, they can lose their RNA, ribosomes, and remaining intracellular organelles.

It was hypothesized that circulating reticulocytes might be the source hemoglobin transcripts enriched in the pS6 IPs. Although visible blood vessels were removed when dissecting the hypothalamus, there are numerous capillaries within the brain parenchyma, and blood from these capillaries can contaminate hypothalamic lysates. To test whether the hemoglobin transcripts originated from circulating cells, mice were transcardially perfused with saline prior to dissection, and then quantified the amount of hba-a1 and hbb-b1 mRNA remaining in hypothalamic extracts. Perfusion reduced by approximately 95% the amount of hba-a1 and hbb-b1 RNA in the hypothalamus, but had no effect on transcripts that are expressed in hypothalamic neurons, such as actin (bact) or pomc (see FIG. 13C). Thus, at least about 95% of hba-a1 and hbb-b1 mRNA in the brain can originate from circulating cells.

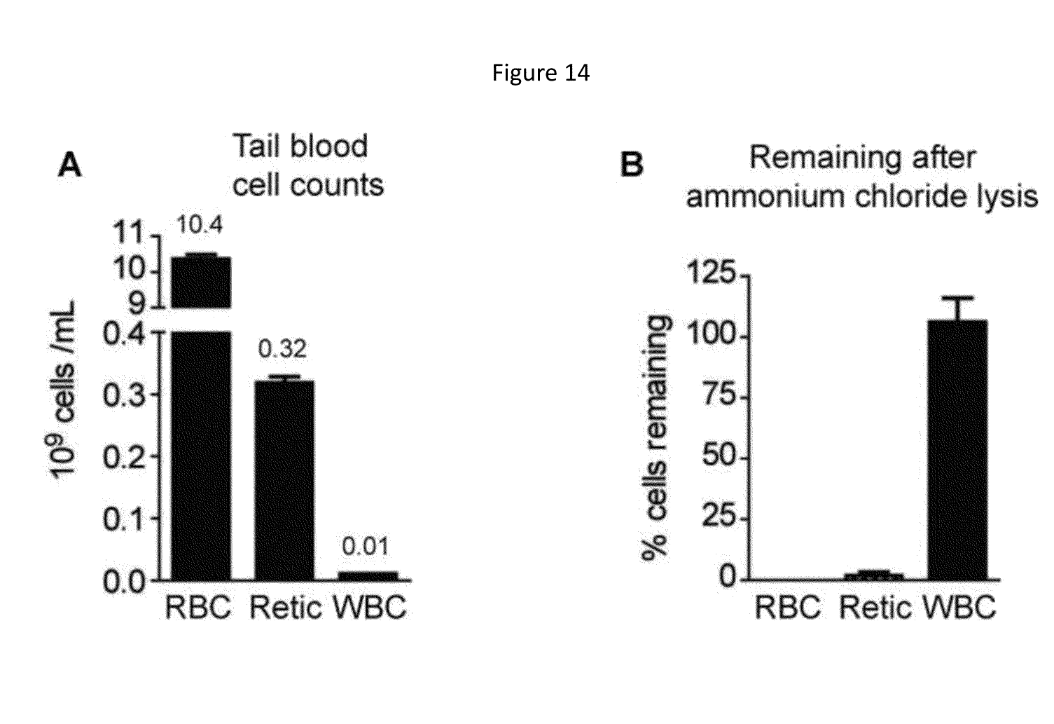

To test whether reticulocytes indeed have high levels of pS6, peripheral blood was isolated and washed, selectively lysed the red blood cells (including reticulocytes) using ammonium chloride, and then separated these red blood cell lysates from the remaining cells by centrifugation. Peripheral blood was washed with HBSS+20 mM EDTA, and then divided into two equal parts. One part was resuspended in ammonium chloride lysis solution and the other was resuspended in HBSS+20 mM EDTA. Both resuspensions were incubated for 20 min on ice. The ammonium chloride lysis but not HBSS caused the resuspended blood to become clear within 5 minutes. Both sets of cells were then collected by centrifugation, the supernatant removed, and the pellets resuspended in equal volumes of HBSS+20 mM EDTA. The resuspensions were then counted using an Advia 120 Hematology Analyzer. Plotted in FIG. 14B is the percentage of cells remaining after ammonium chloride lysis relative to the number remaining after incubation with HBSS.

It was confirmed by automated cell counting (FIG. 14A) that this procedure quantitatively lyses reticulocytes but has no effect on white blood cells (FIG. 14B). The level of pS6 was then measured by western blotting in lysates from reticulocytes versus the hypothalamus as a whole (FIG. 13D). This revealed that reticulocytes can have a much higher stoichiometry of S6 phosphorylation than the hypothalamus, and this was quantified by densitometry (FIG. 13E). Thus, the enrichment of globin transcripts in the hypothalamic pS6 immunoprecipitates can be the result of contamination by reticulocytes, a cell type that was found to have unusually high levels of pS6.

The potential link between mTORC1 signaling and red blood cells was intriguing, because a common side-effect of rapamycin therapy in humans can be microcytic anemia (a decrease in red blood cell size) (see, e.g., Sofroniadou and Goldsmith (2011) Drug Safety 34, 97-115 and Sofroniadou et al. (2010) Nephrol Dial Transplant 25, 1667-1675; each of which is hereby incorporated by reference in its entirety). The cause of rapamycin induced anemia is unknown, and mTORC1 signaling in reticulocytes has not been extensively investigated. However, because the primary function of reticulocytes is to synthesize hemoglobin, it is plausible that these cells would have elevated demand for mTORC1 signaling in order to stimulate protein translation.

In addition to rapamycin, dietary iron deficiency can also cause microcytic anemia. As mTORC1 can be regulated by nutrient availability, it was hypothesized whether mTORC1 signaling in reticulocytes might be sensitive to dietary iron. Mice that had been maintained on a standard chow diet was taken (220 ppm iron) and switched them to a low iron diet (2-6 ppm iron) for 4 weeks. To hasten the development of iron deficiency, the mice additionally received subcutaneous injections of deferoxamine (DFO), a clinically approved iron chelator, for the final 10 days of the experiment.

This protocol induced characteristic features of anemia in mice, including a decrease in the volume of reticulocytes and mature RBCs (quantified in FIG. 13F) and a decrease in the amount of hemoglobin per cell (15.3 versus 13.2 pg/cell in reticulocytes, p<0.001). Moreover, the fraction of cells that scored as having low hemoglobin increased significantly in response to iron deficiency in both cell types (quantified in FIG. 13G). Note that all of these effects are more pronounced in reticulocytes possibly because of their faster turnover relative to mature RBCs (<7 versus 40-50 days). Reticulocyte lysates were prepared from iron deficient mice and mice on a standard diet and compared the levels of pS6 by western blotting. It was found that iron deficiency indeed reduced levels of pS6 in reticulocytes (see western blot in FIG. 13H), suggesting that the availability of iron can regulate mTORC1 signaling in these cells.

The effect of iron on pS6 in reticulocytes could be a direct effect of iron sensing in reticulocytes or a secondary effect of other metabolic changes that accompany anemia. Although iron is not a classical input into the mTOR pathway, iron chelation can inhibit mTORC1 signaling (see, e.g., Ndong et al. (2009) Nutr Res 29, 640-647 and Ohyashiki et al. (2009) Cancer Sci. 100, 970-977; each of which is hereby incorporated by reference in its entirety). K562 cells, an erythroleukemia cell line that expresses alpha globin, were treated with two structurally unrelated iron chelators, DFO and deferasirox (DFS). Both compounds substantially reduced phosphorylation of S6 at 235/236 and 240/244, confirming that iron deficiency can cell autonomously inhibit mTORC1 signaling (FIG. 13I). As iron deficiency anemia is characterized by a decrease in red blood cell size, these data suggest that reduced mTORC1 signaling in reticulocytes may play a previously unappreciated role in this condition.

Reticulocytes have High Levels of S6 Phosphorylation

Large scale studies of gene expression in the mouse brain, such as the Gensat project and the Allen Brain Atlas, have revealed an extraordinary degree of anatomical heterogeneity in neuronal gene expression (see, e.g., Gong et al. (2003) Nature 425, 917-925 and Lein et al. (2007) Nature 445, 168-176; each of which is hereby incorporated by reference in its entirety). The scale of this complexity is such that even reliable estimates for the number of cell types in many regions of the brain was lacking (see, e.g., Masland (2004) Curr Biol 14, R497-500; Nelson et al. (2006) Trends Neurosci 29, 339-345; and Stevens (1998) Curr Biol 8, R708-710; each of which is hereby incorporated by reference in its entirety). Similarly, progress toward assigning the various functions of the brain to individual cell types is at an early stage. This objective remains unmet in part because there are no general methods for discovering molecular markers that describe populations of activated neurons. Tools such as flow cytometry, which have enabled immunologists to parse the cellular diversity of the hematopoietic system, are much more difficult to apply to the adult mammalian brain, where projection neurons can be damaged by the simple process of disaggregation (see, e.g., Emery and Barres (2008) Cell 135, 596-598; which is hereby incorporated by reference in its entirety). For this reason, methods for the physical separation of cell-types have not been as widely adopted in neuroscience as in other fields.

It was noticed that the functional activation of neurons often correlates with stimulation of mTORC1 signaling (see, e.g., Villanueva et al. (2009) Endocrinology 150, 4541-4551; which is hereby incorporated by reference in its entirety), and specifically phosphorylation of S6, suggesting a direct way to affinity-purify ribosomes from those cells. This approach was used to explore neuronal activation in the hypothalamus, in part due to the numerous functionally defined cell-types in this region. It was confirmed that pS6 immunoprecipitation enriches for markers for functionally activated cells, often as the single most highly enriched transcript when the entire genome is ranked according to fold-enrichment, and these markers were validated by immunohistochemistry in multiple cases. It was surprising to find that neuropeptides, which are widely-used to identify the cell types of the hypothalamus, were repeatedly identified as the most enriched genes in these experiments. This suggests that these functional proteins may indeed represent the most cell-type specific genes expressed in a number of functionally defined neuronal populations.

The approach described herein is referred to as phosphoTRAP, in analogy to recently developed approaches such as BacTRAP that use tagged ribosomes to profile translation in sparse populations of neurons (see, e.g., Heiman et al. (2008) Cell 135, 738-748 and Sanz et al. (2009) PNAS106, 13939-13944; each of which is hereby incorporated by reference in its entirety). Whereas BacTRAP relies on bacmid transgenic mice to deliver epitope-tagged ribosomes to specific cell types, the fact that the mTOR pathway has evolved to deliver a phosphorylation tag to the ribosome in functionally activated cells was exploited. For this reason, phosphoTRAP uniquely enables the unbiased identification of genetic markers that describe an activated population of cells.

The sensitivity of this method is illustrated by the discovery that the cells in the brain with the highest level of pS6 are, in fact, red blood cells. However, because pS6 is present at a low basal level in almost all cells, the degree of cell-specific mRNA enrichment that can be achieved with this approach is determined by the dynamic range of pS6 in the tissue being studied. It is estimate that this is ˜10-fold in the mouse brain, based in part on the magnitude of the changes in pS6 that was observed by imaging. BacTRAP, on the other hand, requires prior knowledge of a promoter that marks the relevant population of cells. But once this is known, transgenic mice can be generated that enable higher levels of cell-specific mRNA enrichment. For this reason these two approaches are viewed as complementary, with phosphoTRAP enabling the hypothesis-free identification of markers for functionally activated cells, and BacTRAP enabling a deeper exploration of the genes expressed in those cells.

The finding that reticulocytes have high levels of S6 phosphorylation has potential implications for the pathogenesis of the microcytic anemia associated with rapamycin treatment and iron deficiency. While the clinical use of rapamycin as an immunosuppressant has motivated numerous studies into mTOR signaling in white blood cells, relatively little is known about the role of mTOR in red blood cell development. The discovery that reticulocytes have high basal levels of pS6 called the attention to mTORC1 signaling in these cells, which was found to be regulated by dietary iron. As anemia is a disease that can be caused by a decrease in cell size, these observations suggest a plausible connection between mTORC1 signaling in reticulocytes and anemia. In this regard, it is worth noting that reticulocytes are already known to express a kinase, heme regulated eIF2alpha kinase (HRI), that is regulated by iron and controls translation through the phosphorylation and inhibition of eIF2alpha (see, e.g., Chen (2007) Blood 109, 2693-2699; which is hereby incorporated by reference in its entirety). In other cell types eIF2alpha kinases act in parallel to the mTOR pathway to regulate translation in response to signals such as amino acid availability and stress. The data suggest that, in reticulocytes, the mTOR pathway and eIF2alpha kinases may likewise function in parallel to regulate translation in response to the availability of iron.

In this study, pS6 was used as a tag to mark ribosomes from cells with active mTORC1 signaling. The biochemical function of phosphorylation of S6 remains unknown, despite the fact that numerous studies have reported measurements of pS6 as a surrogate for mTORC1 kinase activity. It was proposed over 40 years ago that phosphorylation of S6 may alter the affinity of the ribosome for a subset of RNAs such as those involved in cell growth and proliferation (see, e.g., Gressner and Wool (1974) J Biol Chem 249, 6917-6925 and Kabat (1970) Biochemistry 9, 4160-4175; each of which is hereby incorporated by reference in its entirety). While this hypothesis remains widely cited, the putative mRNAs that are selectively translated in response to S6 phosphorylation have not been identified (see Meyuhas (2008) Int Rev Cell Mol Biol 268, 1-37; which is hereby incorporated by reference in its entirety).

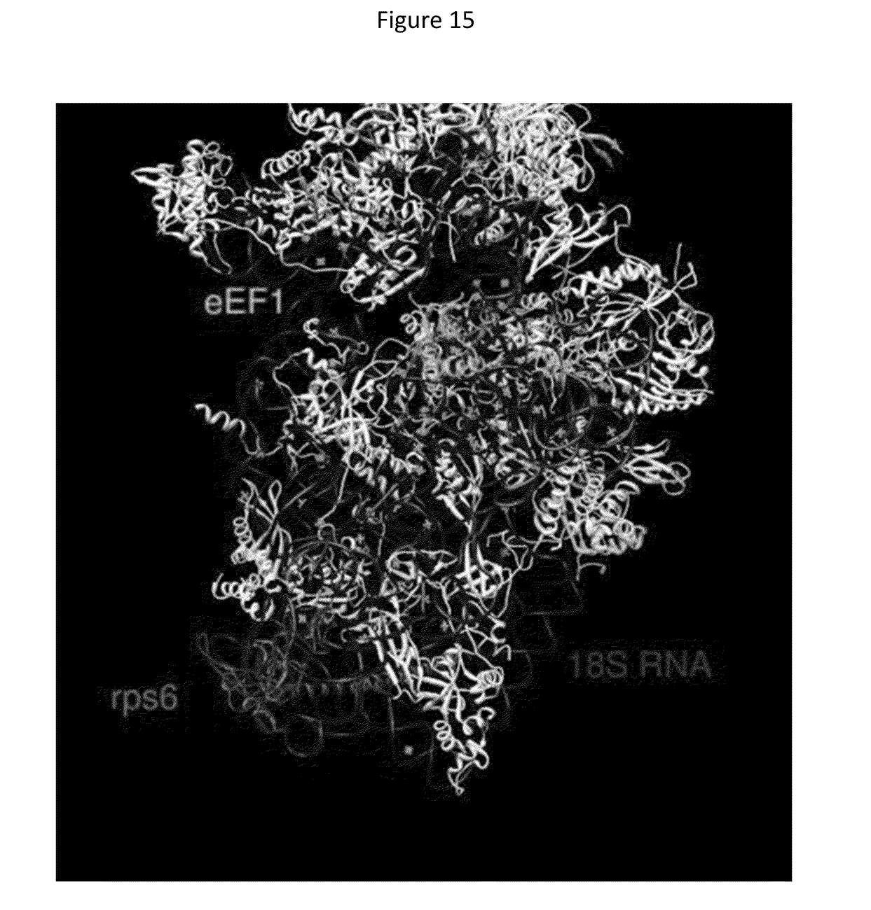

A genome-wide analysis was performed of the mRNAs that are bound to pS6 ribosomes in the mouse brain, a tissue in which most cells have a relatively low level of pS6 at baseline. If a subset of mRNAs involved cell growth or proliferation were strongly biased for or against association with pS6 ribosomes, it would be expected to see these genes consistently enriched or depleted in pS6 immunoprecipitates. In fact no evidence was found for a class of mRNAs that are strongly biased in this way (>2-fold). Rather, it was found that the most highly enriched or depleted mRNAs in the immunoprecipitates were typically genes with cell-type restricted expression, and immunohistochemistry was used to confirm in many cases that the enrichment of these mRNAs can be explained by the stoichiometry of S6 phosphorylation in the cells in which they are expressed. This finding is also supported by the recent crystal structure of the eukaryotic 40S ribosomal subunit, which revealed, contrary to expectations, that the phosphorylation sites on S6 are distant from the ribosome decoding site (see, e.g., Rabl et al. (2011) Science 331, 730-736; which is hereby incorporated by reference in its entirety) (See FIG. 15). Without wishing to be bound by any particular theory, it is believed that S6 phosphorylation plays a role other than the recruitment of specific mRNAs to the ribosome.

Nonetheless, the possibility that S6 phosphorylation has smaller effects on ribosome recruitment or that it regulates translation of specific messages in a more complex way cannot be excluded. Such effects might be masked in a tissue such as the brain, which contains a heterogeneous population of post-mitotic cells. A definitive answer to this longstanding question could require the application of approaches for genome-wide ribosome footprinting—such as the deep sequencing of ribosome protected fragments (see, e.g., Ingolia et al. (2009) Science 324, 218-223; which is hereby incorporated by reference in its entirety)—to the analysis of cells from knock-in mice that lack phosphorylation of S6.

The ribosome has a unique role in biology as the physical platform that connects genotype to phenotype. The data reveal that subpopulations of ribosomes encode an extraordinary amount of information about the organization of biological systems, a finding consistent with the recent work of others (see, e.g., Heiman et al. (2008) Cell 135, 738-748; Hendrickson et al. (2009) PLoS Biol 7, e1000238; Ingolia et al. (2009) Science 324, 218-223; and Sanz et al. (2009) PNAS106, 13939-13944; each of which is hereby incorporated by reference in its entirety). The present experiments focused on the phosphorylation of S6, which has been studied for decades, but less is known about the other features that define functional populations of ribosomes in the cell. It is believed that the identification of these pools of ribosomes and their associated transcripts is a continuing source of biological insights.

### Example 2

### Molecular Profiling of Activated Neurons by Phosphorylated Ribosome Capture

The mammalian brain is composed of thousands of interacting neural cell-types. Systematic approaches to establish the molecular identity of functional populations of neurons would advance the understanding of neural mechanisms controlling behavior. The results presented herein show that ribosomal protein S6, a structural component of the ribosome, can become phosphorylated in neurons activated by a wide-range of stimuli. The results show that these phosphorylated ribosomes can be captured from mouse brain homogenates, thereby enriching directly for the mRNAs expressed in discrete subpopulations of activated cells. This approach was used to identify neurons in the hypothalamus that can be regulated by changes in salt balance or food availability. It was observed that galanin neurons can be activated by fasting and that prodynorphin neurons can restrain food intake during scheduled feeding. These studies identify new elements of the neural circuit that can control food intake and illustrate how the activity-dependent capture of cell-type specific transcripts can help elucidate the functional organization of a complex tissue.

A goal of neuroscience is to link the activity of specific neuronal cell-types to the various functions of the brain. This task can be complicated by the extraordinary cellular diversity of the mammalian CNS (Lichtman, J. W. & Denk, W. Science 334, 618-623, doi:10.1126/science.1209168 (2011); Stevens, C. F. Curr Biol 8, R708-710 (1998); Masland, R. H. Curr Biol 14, R497-500, doi:10.1016/j.cub.2004.06.035 (2004); and Nelson, S. B., Sugino, K. & Hempel, C. M. Trends in neurosciences 29, 339-345, doi:10.1016/j.tins.2006.05.004 (2006); each of which is incorporated by reference in its entirety), and the fact that many neurons cannot be identified based solely on their morphology or location (Lein, E. S. et al. Nature 445, 168-176, doi:10.1038/nature05453 (2007); Yizhar, O., et al. Neuron 71, 9-34, doi:10.1016/j.neuron.2011.06.004 (2011); Isogai, Y. et al. Nature 478, 241-245, doi:10.1038/nature10437 (2011); Morgan, J. I., et al. Science 237, 192-197 (1987); and Morgan, J. I. & Curran, T. Annual review of neuroscience 14, 421-451, doi:10.1146/annurev.ne.14.030191.002225 (1991); each of which is incorporated by reference in its entirety). Comprehensive analyses of gene expression in the nervous system, such as the GENSAT project and the Allen Brain Atlas, have revealed extensive heterogeneity in gene expression across brain regions (Gong, S. et al. Nature 425, 917-925, doi:10.1038/nature02033 (2003) and Lein, E. S. et al. Nature 445, 168-176, doi:10.1038/nature05453 (2007); each of which is incorporated by reference in its entirety), but there are significant gaps in the understanding of how this molecular diversity is linked to function.

The molecular identification of neural populations that are modulated by a stimulus would advance the understanding of the functional organization of the brain, and can provide for the use of new technologies that can make it possible to manipulate rare populations of neurons in vivo. These tools can include optogenetic reagents for the activation or inhibition of neurons with light (Yizhar, O., et al. Neuron 71, 9-34, doi:10.1016/j.neuron.2011.06.004 (2011), which is incorporated by reference in its entirety); collections of transgenic mice that express GFP in specific cell populations, which can enable their identification for recording (Gong, S. et al. Nature 425, 917-925, doi:10.1038/nature02033 (2003), which is incorporated by reference in its entirety); and methods for generating transcriptional profiles from individual cell-types using tagged ribosomes (Heiman, M. et al. Cell 135, 738-748, doi:10.1016/j.ce11.2008.10.028 (2008), which is incorporated by reference in its entirety). These tools can achieve a level of specificity by targeting protein expression to an individual cell-type using the promoter from a marker gene. However, the genes that identify a functional population of neurons can be unknown (Zhang, F., et al. Nature reviews. Neuroscience 8, 577-581, doi:10.1038/nrn2192 (2007), which is incorporated by reference in its entirety). Characterizing the co-expression of even a limited set of marker genes can require processing large numbers of histologic sections (Isogai, Y. et al. Nature 478, 241-245, doi:10.1038/nature10437 (2011), which is incorporated by reference in its entirety). This problem persists in part because there is a lacking in systematic methods to profile gene expression from discrete subpopulations of activated neurons in the brain.

The results presented wherein show that phosphorylation of the ribosome can be used as a molecular tag to retrieve RNA selectively from activated neurons. This can enable the unbiased discovery of the genes that are expressed in a functional population of neurons. By quantifying in parallel the enrichment of many such markers, it is possible to assess the activation or inhibition of each cell-type in a tissue, which can reveal the coordinated regulation of ensembles of neurons in response to an external stimulus. In this example, this approach was used to identify new components of the neural circuit that controls feeding in the hypothalamus.

The materials and methods employed in these experiments are now described.

### Materials

The following antibodies were used: rabbit anti-pS6 240/244 (Cell Signaling #2215), rabbit anti-pS6 235/236 (Cell Signaling #4858), rabbit anti-rpL26 (Novus Biologicals, NB100-2131), rabbit anti-rpL7 (Novus Biological, NB100-2269), mouse anti-oxytocin (Millipore, MAB5296; 1:1000), guinea pig anti-vasopressin (Peninsula Laboratories, 1:3000), chicken anti-GFP (Abeam, ab13970; 1:1000), rabbit anti-FosB (Cell Signaling, #2251, 1:25), rabbit anti-CXCL1 (Abcam,ab17882; 1:200), mouse anti-rpS6 (Cell Signaling, #2317, 250 ng/mL), rabbit anti-c-fos (Santa Cruz, sc-52, 1:2000). The following mice used in this study are available from Jackson laboratories (Cat. #): POMC-eGFP (009593), Tsc1^{fl/fl} (005680), NPY-hrGFP (006417), Rosa26-YFP (006148), Sim1-Cre (006395), Pmch-eGFP (008324), Fos-eGFP (014135), and Lepr-Cre (008320). CRH-eGFP mice have been described⁵⁰, and the Pmch-Cre mice will be described separately. The 3P peptide was synthesized by United Peptide and has the sequence biotin-QIAKRRRLpSpSLRApSTSKSESSQK where pS is phosphoserine (SEQ ID NO:25). S6^{S5A} and wild-type MEFs were a gift from David Saatini.

### Ribosome Immunoprecipitations

Protein A Dynabeads (150 μL, Invitrogen) were loaded with 4 μg of pS6 antibody (Cell Signaling #2215) in Buffer A (10 mM HEPES [pH 7.4], 150 mM KCl, 5 mM MgCl₂, 1% NP40, 0.05% IgG-free BSA) at 4 C. Beads were washed three times with Buffer A immediately before use.

Mice were sacrificed by cervical dislocation. The hypothalamus was rapidly dissected in Buffer B on ice (1×HBSS, 4 mM NaHCO₃, 2.5 mM HEPES [pH 7.4], 35 mM Glucose, 100 μg/mL cycloheximide). Hypothalami were pooled (typically 5-20 per IP), transferred to a glass homogenizer (Kimble Kontes 20), and resuspended in 1.35 mL of buffer C (10 mM HEPES [pH 7.4], 150 mM KCl, 5 mM MgCl₂, 100 nM calyculin A, 2 mM DTT, 100 U/mL RNasin, 100 μg/mL cycloheximide, protease and phosphatase inhibitor cocktails). Samples were homogenized three times at 250 rpm and nine times at 750 rpm on a variable-speed homogenizer (Glas-Col) at 4° C. Homogenates were transferred to a microcentrifuge tube and clarified at 2000×g for 10 min at 4° C. The low-speed supernatant was transferred to a new tube on ice, and to this solution was added 90 μL of 10% NP40 and 90 μL of 1,2-diheptanoyl-sn-glycero-3-phosphocholine (DHPC, Avanti Polar Lipids: 100 mg/0.69 mL). This solution was mixed and then clarified at 17000×g for 10 min at 4° C. The resulting high-speed supernatant was transferred to a new tube, and 20 μL of a 0.05 mM stock solution of 3P peptide was added. A 20 μL aliquot of this solution was removed, transferred to a new tube containing 350 μL buffer RLT (Qiagen), and stored at −80° C. for purification as input RNA. The remainder was used for immunoprecipitation.

Immunoprecipitations were allowed to proceed 10 min at 4° C. The beads were then washed four times with buffer D (10 mM HEPES [pH 7.4], 350 mM KCl, 5 mM MgCl₂, 2 mM DTT, 1% NP40, 100 U/mL RNasin, and 100 μg/mL cycloheximide). During the third wash the beads were transferred to a new tube and allowed to incubate at RT for 10 min. After the final wash the RNA was eluted by addition of buffer RLT (350 μL) to the beads on ice, the beads removed by magnet, and the RNA purified using the RNeasy Micro Kit (Qiagen). RNA assessed using an Agilent 2100 bioanalyzer. For microarray analysis, cDNA was prepared using the Ovation RNA Amplification System V2 (NuGEN), and hybridized to MouseRef-8 v2 BeadChips (Illumina) For RNA-seq analysis, cDNA was prepared using the SMARTer Ultralow Input RNA for Illumina Sequencing Kit (634935, Clontech) and then sequenced using an Illumina HiSeq 2000.

### Cell Culture Experiments

Wild-type and S6^{S5A} MEFs were cultured in DMEM supplemented with 10% FBS and penicillin-streptomycin. Cells were grown to confluence, starved for 6 hours in 0.25% FBS/DMEM, and restimulated with 20% FBS/DMEM supplemented with 100 nM insulin for 30 minutes. Cells were washed with PBS, trypsinized, collected by centrifugation, and then lysed in a 1% NP40 buffer containing protease and phosphatase inhibitors. Lysates were clarified, immunoprecipitated using pS6 antibodies, and the recovered RNA quantified using an Agilent Bioanalyzer.

For microarray analysis of Hepa1-6 and NIH3T3 cells (Supplementary FIG. 3), subconfluent cells were grown overnight in DMEM supplemented with 10% FBS. The following day the media was removed, the cells were washed with PBS, and then lysed by direct addition of buffer D supplemented with protease and phosphatase inhibitors. Lysates were clarified and immunoprecipitated using pS6 antibodies. The recovered RNA analyzed using Illumina Beadchips as described above.

### Animal Treatment

Wild-type male C57B6/J mice from Jackson laboratories were maintained on a 8 pm:8 am light-dark schedule and were 9-13 weeks old at the time of sacrifice. All dissections were performed between noon and 2 pm except as noted. For osmotic stimulation experiments, animals were given an intraperitoneal injection of 2M NaCl (350 μL), water was removed from the cage, and mice were sacrificed 120-140 min later. For fasting experiments, animals were transferred to a new cage without food at 5 pm and then sacrificed at 8 am the following morning. Control mice were fed ad libitum and sacrificed at the same time. For ghrelin experiments, animals were given an intraperitoneal injection of ghrelin (66 μg, Tocris), food was removed from the cage, and animals were dissected 70 min later. For scheduled feeding, animals were allowed access to food between noon and 3 pm each day, and then sacrificed between 1:45 and 2 pm after a minimum of 10 days on this schedule.

For drug treatments, mice were given an intraperitoneal injection of the following dose and then sacrified by transcardial perfusion with saline at the indicated time: cocaine (30 mg/kg, 60 min), kainate (12.5 mg/kg, 120 min), haloperidol (2 mg/kg, 30 min), olanzapine (20 mg/kg, 120 min), clozapine (10 mg/kg, 45 min) For cat odor experiments, a domestic cat was fitted with a fabric collar (Safe Cat) for three weeks; the collar was removed, mice were exposed to the collar for 60 min, and then sacrificed by perfusion. For the resident intruder test, a male mouse was single caged for at least two weeks, a male conspecific was introduced into the cage, and the animals were monitored for the number and latency of attacks. The resident mouse was then perfused after 60 min. For dehydration experiments, water was removed from the cage and mice were perfused 24 h later.

Treatment with KOR Antagonists During Scheduled Feeding

JDTic was either delivered by intraperitoneal injection (10 mg/kg) or was reconstituted in PBS to a concentration of 1 mg/mL and 5 ul was delivered via Hamilton syringe into the lateral ventricle using coordinates: L/M 1.0 mm from Bregma, A/P −0.4 mm from Bregma and 2.5 mm beneath the cortex. Norbinaltorphimine was delivered at the same dose and coordinates as described above.

### Immunohistochemistry

Mice were sacrified at the indicated times by isoflurane anesthesia followed by transcardial perfusion with PBS and then 10% formalin. Brains were dissected, incubated in 10% formalin overnight at 4° C., and 40 μm sections were prepared on a vibratome. Free floating sections were blocked for 1 h at room temperature in buffer E (PBS, 0.1% Triton, 2% goat serum, 3% BSA), and then stained overnight at 4° C. with primary antibodies at the indicated concentrations. For pS6 244 staining, the pS6 240/244 polyclonal antibody (Cell Signaling, #2215) was combined with the 3P peptide (250 nM final concentration). The following day sections were washed with PBS+0.1% Triton (3×20 min); incubated with dye-conjugated secondary antibodies at 1/1000 for 1 h at room temperature, washed in PBS+0.1% Triton (3×20 min), and then mounted. For AVP immunostaining, it was noted that goat anti-rabbit secondary antibodies cross-react with guinea pig primary antibodies; therefore primary antibody incubations were performed sequentially. For FosB staining, primary antibody incubations were allowed to proceed for 72 h.

### Fluorescent In Situ Hybridization for Galanin and Pdyn

For galanin, a 633 base pair anti-sense digoxigenin-labeled riboprobe were synthesized chemically. For prodynorphin, a 592 base pair anti-sense digoxigenin-labeled riboprobe were synthesized chemically. 40 μm vibratome free-floating sections were incubated in 3% H₂O₂ for 1 h at room temperature to quench endogenous peroxidase activity. Sections were treated with 0.20% acetic anhydride followed by 1% Triton-X for 30 min each. Prehybridization was carried out at 37° C. using hybridization buffer (50% formamide, 5×SSC, 5×Denhardts, 250 ug/mL baker's yeast RNA, 500 ug/mL ssDNA) for 1 h before overnight hybridization with riboprobe at 62° C. Sections were washed in 5×SSC followed by 2 washes with 0.2×SSC at 62° C. Brief washes with 0.2×SSC and buffer B1 (0.1M Tris pH 7.5, 0.15M NaCl) were performed and sections were blocked in TNB (1% blocking reagent in B1, Roche #1096176) for 1 h at room temperature. Anti-digoxigenin-POD antibody (1:100, Roche #11207733910) was applied overnight at 4° C. Riboprobe was developed using the TSA Plus Fluorescence System (Perkin Elmer, #NEL744) according to the manufacturer's instructions.

### Microscopy and Quantification

Images were acquired using an LSM510 laser scanning confocal microscope. pS6 was quantified in specific neuronal populations as follows. Sections were double immunostained for pS6 244 and the relevant neuropeptide (Avp, Oxt) or neuropeptide GFP mouse (POMC-GFP, AgRP-Cre/Rosa26-YFP, CRH-GFP, Pdyn-GFP). For each of three animals from both experimental and control groups, three sets of Z-stacks were acquired from adjacent sections. The surfaces corresponding to each labelled cell in the field (e.g. each POMC cell) were reconstructed using Imaris software (Bitplane), and the mean intensity in the pS6 channel within the volume bounded by the surface of each labelled cell was recorded. This data was then plotted as a scatter dot plot, with the mean and standard error indicated. Images for comparison in this manner were collected using identical microscope and camera settings on tissue samples processed in parallel. In cases where the absolute number of pS6 positive cells within an anatomic region was desired (e.g. Pdyn neurons in the DMH), the number of pS6 positive and negative cells was counted manually.

### Taqman Array Measurements

Taqman probes were designed and ordered for quantification of each of the 225 genes described in Table 4. Probes were distributed to 96-well plates in duplicate, cDNA was prepared using the Quantitect RT kit (Qiagen), and reactions were run using the Taqman Gene Expression Master Mix (ABI) on an Applied Biosystems 7900HT system. For each experiment (stimulus or control), the abundance of each gene in the input RNA and in the pS6 immunoprecipitated RNA was measured in duplicate. The mean RNA abundance was determined, normalized to an rpL27 probe that was present in every plate, and the ratio (IP/Input) was calculated. The experiment was repeated multiple times for each stimulus or control, these values were averaged, and the differential enrichment was calculated (ratio of (IP/Input) stimulus over control). Note that the differential enrichment was calculated because the neuronal markers that become enriched or depleted specifically in response to the stimulus was of interest. Normalization to the control group accounts for the fact that each neural marker has a somewhat different enrichment at baseline, reflecting the fact that each cell-population has somewhat different level of basal pS6. Follow-up analysis focused in every case on the most highly enriched genes in the experiment, which were validated directly by histology.

The results of the experiments are now described.

Capture of Phosphorylated Ribosomes from Activated Neurons

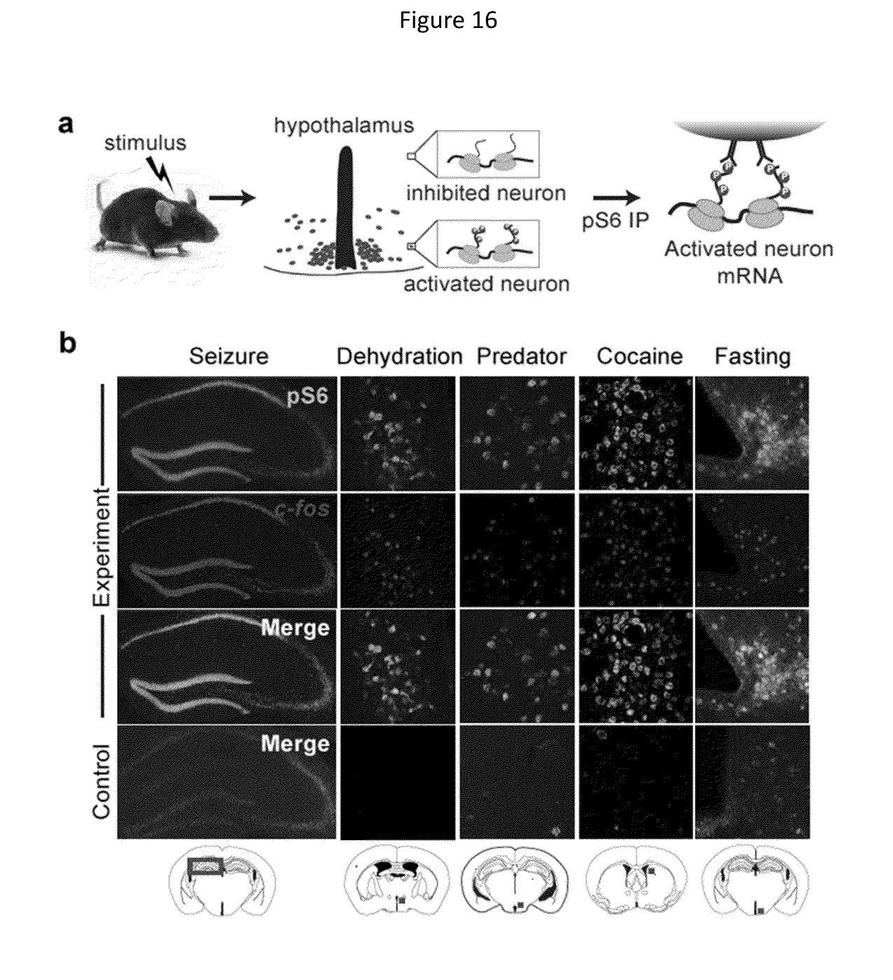

Immediate early genes such as c-fos can be used to visualize activated neurons in the mouse brain (Morgan, J. I., et al. Science 237, 192-197 (1987) and Morgan, J. I. & Curran, T. Annual review of neuroscience 14, 421-451, doi:10.1146/annurev.ne.14.030191.002225 (1991), each of which is hereby incorporated by reference in its entirety), but c-fos staining does not reveal the molecular identity of the labeled cells. Therefore, experiments were designed to develop a method for generating expression profiles from activated neurons. As illustrated in FIG. 16, many stimuli that trigger c-fos expression in activated neurons can induce phosphorylation of ribosomal protein S6 in the same cells (Villanueva, E. C. et al. Endocrinology 150, 4541-4551, doi:10.1210/en.2009-0642 (2009); Cao, R., et al. Molecular and cellular neurosciences 38, 312-324, doi:10.1016/j.mcn.2008.03.005 (2008); Valjent, E. et al. Neuropsychopharmacology 36, 2561-2570, doi:10.1038/npp.2011.144 (2011); and Zeng, L. H., et al. The Journal of neuroscience 29, 6964-6972, doi:10.1523/JNEUROSCI.0066-09.2009 (2009); each of which is hereby incorporated by reference in its entirety). S6 is a structural component of the ribosome that can be phosphorylated downstream of PI3-K/mTOR, MAPK, and PKA signaling (Valjent, E. et al. Neuropsychopharmacology 36, 2561-2570, doi:10.1038/npp.2011.144 (2011) and Meyuhas, O. International review of cell and molecular biology 268, 1-37, doi:10.1016/S1937-6448(08)00801-0 (2008); each of which is hereby incorporated by reference in its entirety). These same pathways can regulate the transcription of activity-dependent genes such as c-fos (Flavell, S. W. & Greenberg, M. E. Annual review of neuroscience 31, 563-590, doi:10.1146/annurev.neuro.31.060407.125631 (2008), which is hereby incorporated by reference in its entirety). It is contemplated that, because S6 phosphorylation introduces a tag on ribosomes that reside in activated neurons, it might be possible to immunoprecipitate these phosphorylated ribosomes from mouse brain homogenates, and thereby enrich for mRNAs expressed in the activated cells (FIG. 16a). By comparing the abundance of each transcript in the pS6 immunoprecipitate to its abundance in the tissue as a whole, it would thus be possible to rank in an unbiased way the genes that are uniquely expressed in a population of activated neurons.

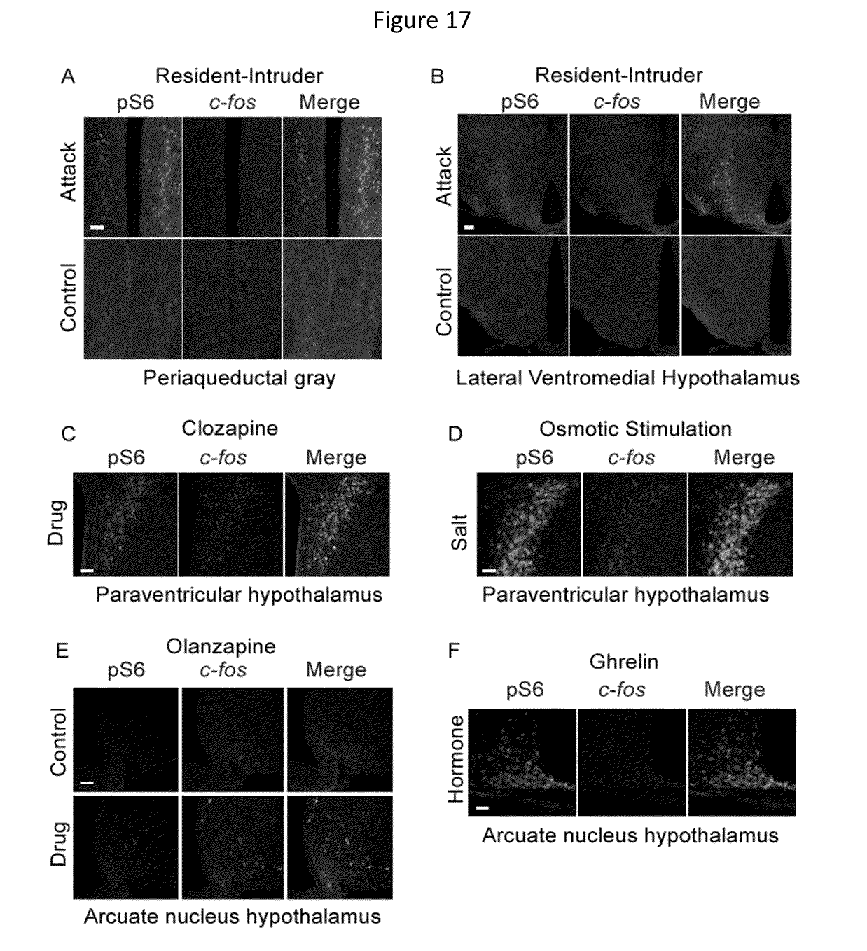

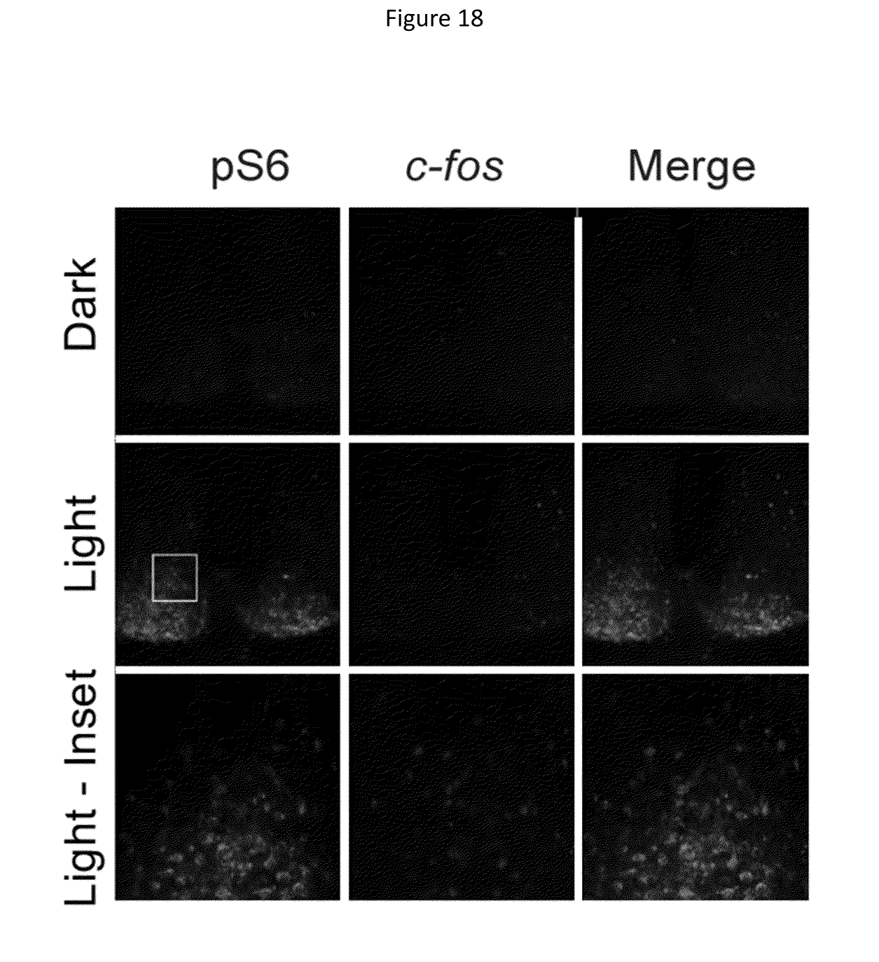

To confirm that S6 was phosphorylated in cells expressing c-fos, mice were exposed to a diverse panel of stimuli and then performed double immunohistochemistry for c-fos and pS6 in brain slices. It was found that treatment of mice with cocaine (a narcotic), kainate (a convulsant), and haloperidol, clozapine and olanzapine (anti-psychotics) can induce co-localization of pS6 and c-fos in a variety of brain regions (e.g., including the hippocampus, striatum, and hypothalamus; FIG. 16b and FIG. 17). Exposure of male mice to an intruder can induce an overlapping pattern of c-fos and pS6 expression in brain regions that are known to mediate aggression (Lin, D. et al. Nature 470, 221-226, doi:10.1038/nature09736 (2011), which is hereby incorporated by reference in its entirety), such as the lateral hypothalamus and periaqueductal gray (FIG. 17). It was found that a cat odorant, which signals to rodents the presence of a predator, induced c-fos and pS6 in regions known to mediate fear and defensive responses, such as the dorsal premammilary nucleus (Dielenberg, R. A., et al. Neuroscience 104, 1085-1097 (2001), which is hereby incorporated by reference in its entirety). (FIG. 16b). A wide variety of other stimuli including fasting, dehydration, leptin deficiency (Villanueva, E. C. et al. Endocrinology 150, 4541-4551, doi:10.1210/en.2009-0642 (2009), which is hereby incorporated by reference in its entirety), and ghrelin treatment also resulted in extensive co-localization of c-fos and pS6 in regions of the hypothalamus that can regulate water and food intake (FIG. 16b and FIG. 17). As illustrated in FIG. 18, one of these markers labeled a broader population of activated neurons than the other; for example, light induced strong pS6 but only scattered c-fos within the suprachiasmatic nucleus (Cao, R., et al. Molecular and cellular neurosciences 38, 312-324, doi:10.1016/j.mcn.2008.03.005 (2008), which is hereby incorporated by reference in its entirety), a region that can regulate circadian rhythms and that can receive input from the retina. However, in general it was found that a wide range of stimuli induced expression of c-fos and pS6 in largely overlapping neural populations throughout the brain.

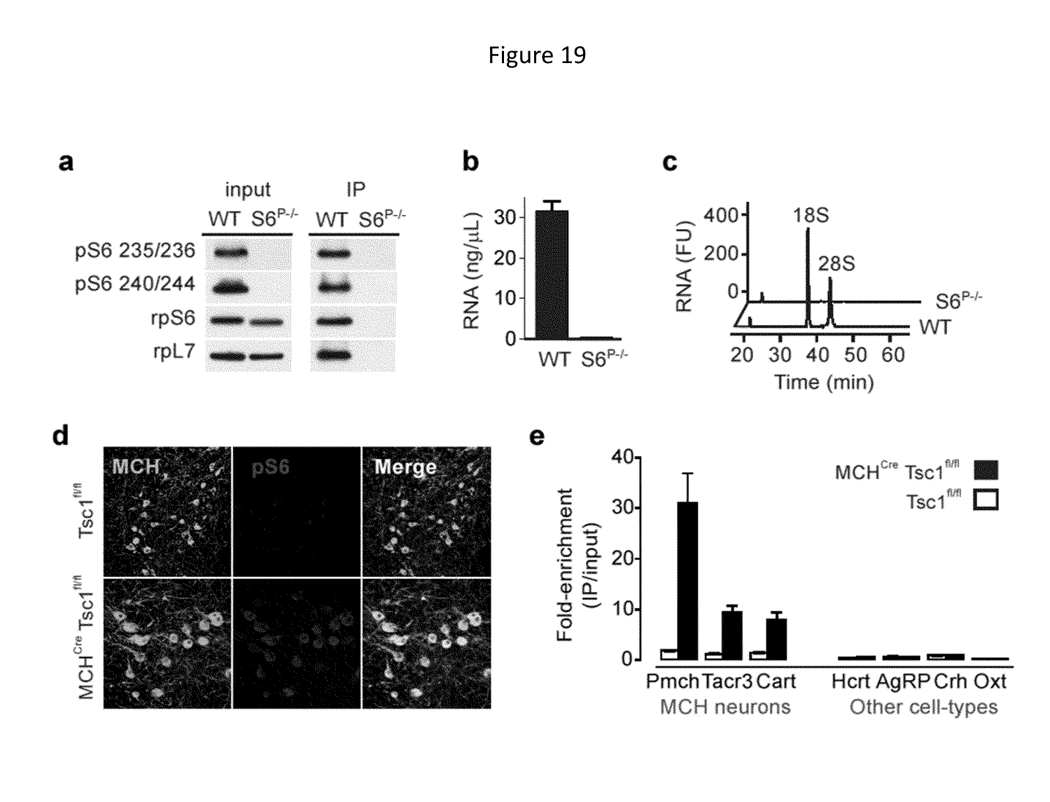

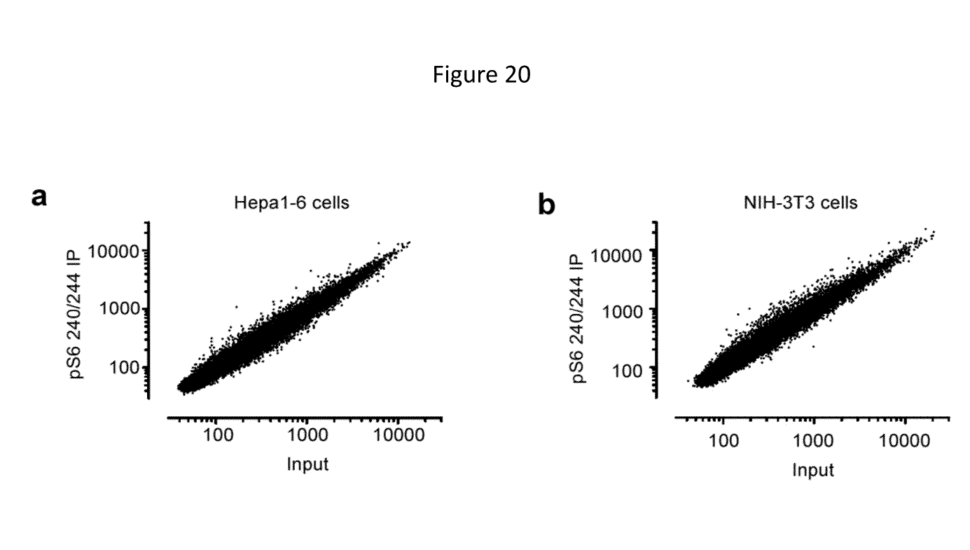

Experiments were designed to to confirm that phosphorylated ribosomes and their associated mRNA could be isolated. Lysates were prepared from wild-type mouse embryonic fibroblasts (MEFs) as well as knock-in MEFs in which each of the five serine phosphorylation sites on S6 was mutated to alanine (Ser235, 236, 240, 244, and 247; S6^{S5A}; Ruvinsky, I. et al. Genes Dev 19, 2199-2211, doi:10.1101/gad.351605 (2005), which is hereby incorporated by reference in its entirety). Antibodies that recognize pS6 240/244 immunoprecipitated ribosomes from lysates of wild-type MEFs but not from S6^{S5A} cells (FIG. 19a). Approximately 100-fold more RNA was isolated in pS6 immunoprecipitates from wild-type MEFs compared to S6^{S5A} controls (FIG. 19b,c), confirming that phosphorylated ribosomes can be captured with high selectivity. Microarray analysis of pS6 immunoprecipitates from cell-lines confirmed that phosphorylated ribosomes associate broadly with entire transcriptome and that the RNAs loaded onto these ribosomes are not strongly enriched or depleted for specific transcripts (FIG. 20).

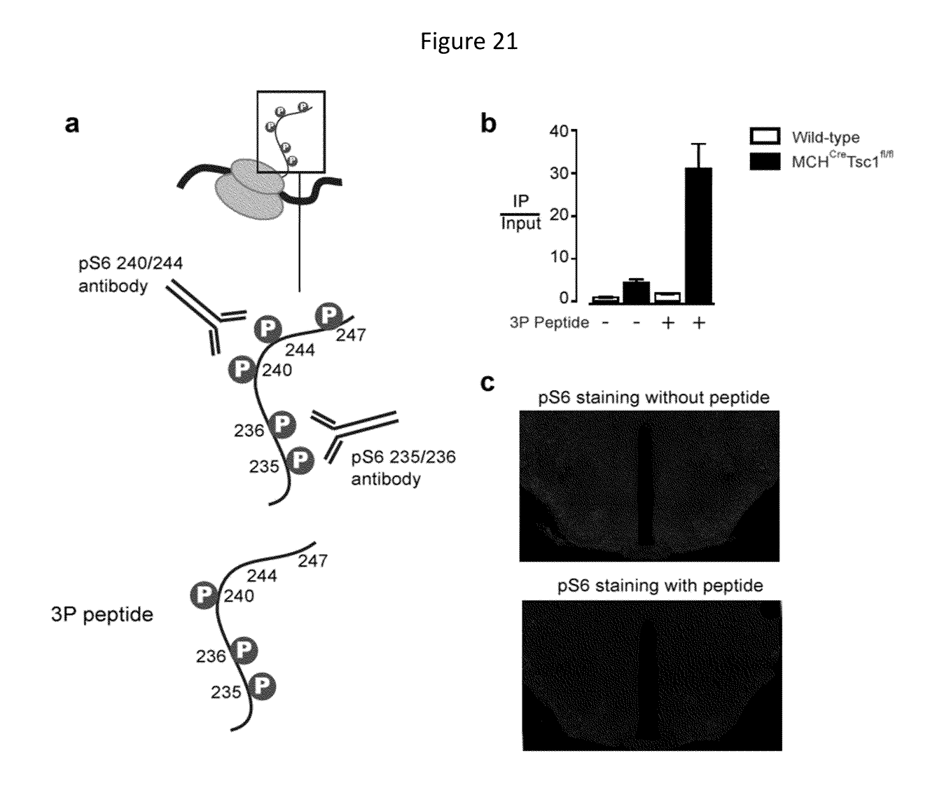

To confirm that mRNA could be enriched from a single neuronal cell-type in vivo, mice in which the gene encoding Tsc1 was selectively deleted in melanin concentrating hormone (MCH) neurons of the lateral hypothalamus (MCH^{Cre} Tsc1n were generated. Tsc1 deletion can result in disinhibition of the mTORC1 pathway and, as illustrated in FIG. 19d, constitutive S6 phosphorylation in the targeted cells (Meikle, L. et al. The Journal of neuroscience 27, 5546-5558, doi:10.1523/JNEUROSCI.5540-06.2007 (2007), which is hereby incorporated by reference in its entirety). Tissue homogenates were prepared from whole hypothalami from these mice, immunoprecipitated phosphorylated ribosomes, and analyzed the purified RNA. In some cases, no more than 4-fold enrichment was achieved for MCH mRNA from MCH^{Cre} Tsc1^{fl/fl} mice using available phosphospecific antibodies that recognize pS6 235/236 or 240/244 (FIG. 21). Because Tsc1 deletion can result in uniform and stoichiometric phosphorylation of S6, this 4-fold enrichment represented an upper limit on the RNA enrichment that could achieve. At this level of enrichment it can be challenging to identify markers for cell-types that underwent graded or heterogenous activation in response to a physiologic stimulus. Ways to capture RNA from activated neurons more selectively were therefore explored.

Phosphorylation of S6 can occur sequentially in the order 236, 235, 240, 244, 247 (Meyuhas, O. International review of cell and molecular biology 268, 1-37, doi:10.1016/S1937-6448(08)00801-0 (2008), which is hereby incorporated by reference in its entirety), such that the most C-terminal sites (244 and 247) can be phosphorylated at much lower stoichiometry than the N-terminal sites at baseline. It was therefore reasoned that phosphorylation of these C-terminal sites could exhibit a wider dynamic range in response to neural activity, and that an antibody recognizing only one of these sites could enable greater enrichment of cell-type specific transcripts. Through extensive empirical testing, it was found that a polyclonal antibody targeting pS6 240/244 could be made more selective by pre-incubation with a phosphopeptide (e.g. an inhibitor peptide) containing the pS6 240 phosphorylation site, thereby generating antibodies that recognize only phosphorylation at 244 (FIG. 21) Immunoprecipitation of phosphorylated ribosomes using these synthetic antibodies resulted in more than 30-fold enrichment of MCH transcripts from MCH^{Cre} Tsc1^{fl/fl} mice but not Tsc1^{fl/fl} controls (FIG. 19e). Robust enrichment (8 to 10-fold) was also observed for genes co-expressed in only a subset of MCH neurons, such as CART and TACR3 (Croizier, S. et al. PLoS One 5, e15471, doi:10.1371/journal.pone.0015471 (2010), which is hereby incorporated by reference in its entirety), but observed no enrichment for genes expressed in a set of different hypothalamic cell-types, such as the neuropeptides HCRT, OXT, AGRP, and CRH (FIG. 19e). Consistent with this qPCR data, brain slices stained using these synthetic antibodies showed enhanced contrast between activated and inactivated neurons compared to slices stained with commercial antibodies that recognize a broader set of phosphorylation sites (FIG. 21c). Thus using this optimized approach allowed for the selective enrichment of the transcripts expressed in neurons with phosphorylated ribosomes in vivo.

### Neurons in the Hypothalamus Regulated by Salt

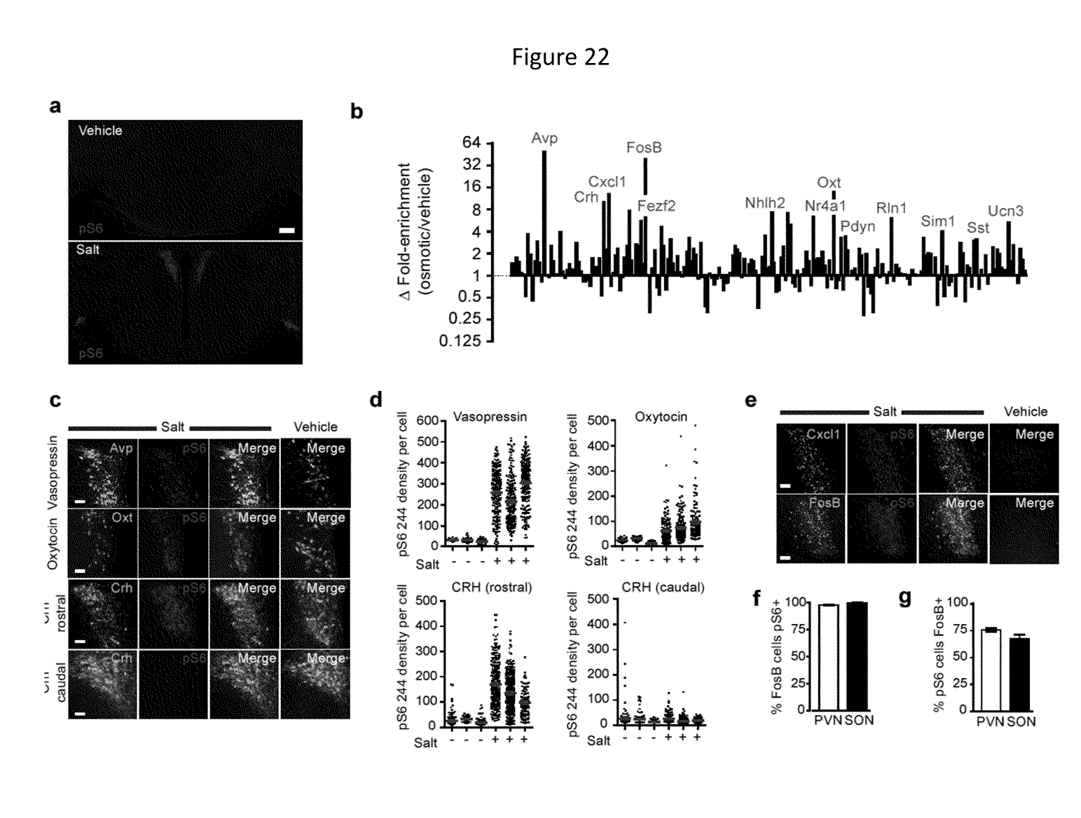

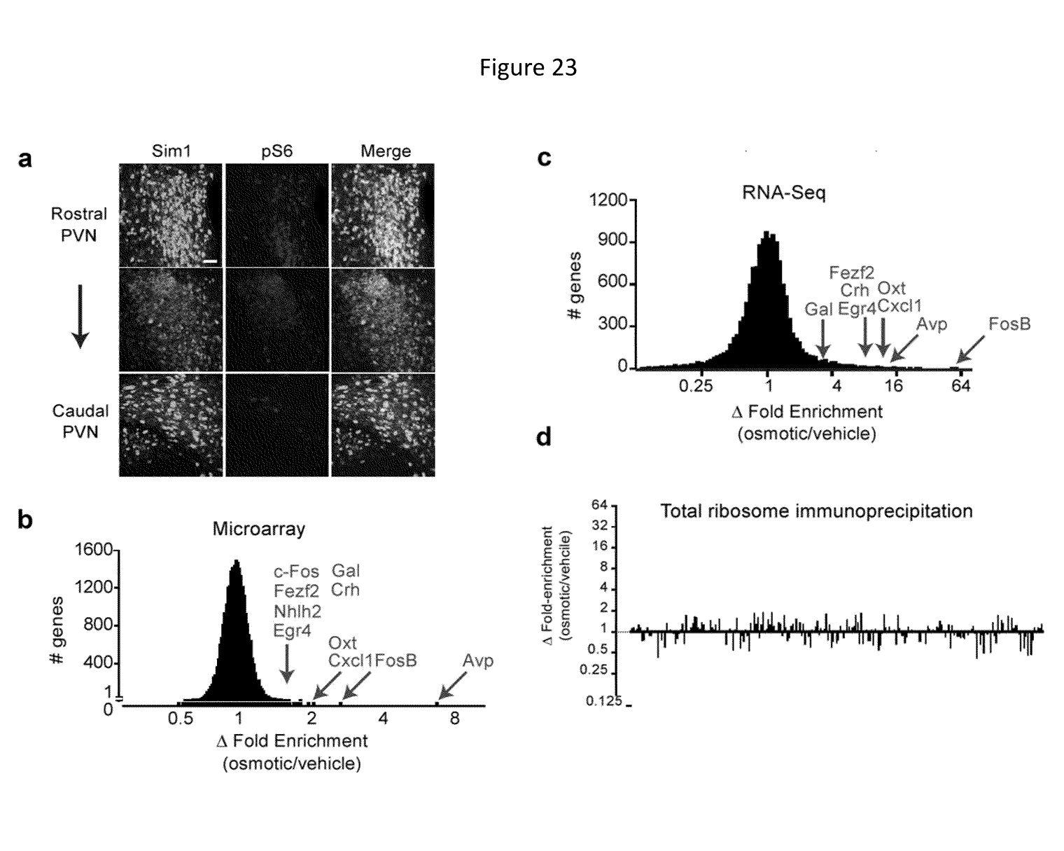

Experiments were designed to identify neurons that were activated in response to a physiologic stimulus. Plasma osmolarity can be controlled by a hypothalamic system that can include vasopressin and oxytocin neurons, and the levels of these peptides can increase in response to salt loading. Mice were challenged with a concentrated salt solution and stained brain sections for pS6 using the aforementioned antibody and blocking peptide. Salt challenge induced an increase in pS6 in regions of the hypothalamus that are known to mediate osmoregulation, including the paraventricular (PVN) and supraoptic nuclei (SON) and median eminence (FIG. 22a). Phosphorylated ribosomes were immunoprecipitated from hypothalamic homogenates of salt-challenged and control animals and analyzed the enriched mRNAs. To enable the rapid and sensitive quantification of low abundance transcripts, a custom array of 225 Taqman probes comprised of marker genes that can show anatomically restricted expression within the hypothalamus was designed. This array includes neuropeptides (80 probe sets) as well as a panel of receptors, transcription factors, and other proteins that mark specific populations of hypothalamic neurons (Table 4). The expression data for these genes is shown as “skyscraper” plots in which the differential enrichment of each gene in response to the stimulus is plotted on a log scale (FIG. 22b). The same enriched genes were also identified using RNA sequencing and microarrays (FIG. 23).

The most highly enriched genes in pS6 immunoprecipitates from salt challenged animals included vasopressin (Avp; 49-fold), oxytocin (Oxt; 14-fold), and corticotropin releasing hormone (Crh; 10-fold) (FIG. 22b and Table 5). Each of these neuropeptides can be expressed in a distinct population of neurons activated by salt loading, and the degree of enrichment of these marker genes correlated with the quantitative induction of pS6 in the corresponding cells (FIG. 22c,d). Enrichment was likewise detected at a lower level for genes known to partially overlap in expression with Avp and Oxt (Gai, W. P., et al. The Journal of comparative neurology 298, 265-280, doi:10.1002/cne.902980302 (1990) and Sherman, T. G., et al. Neuroendocrinology 44, 222-228 (1986), each of which is incorporated by reference in its entirety), such as the neuropeptides galanin (Gal; 4.6-fold) and prodynorphin (Pdyn; 3.4-fold), and the PVN specific transcription factors Nhlh2 (7.3-fold), Fezf2 (5.5-fold), and Sim1 (4.0-fold) (FIG. 22b and FIG. 23). Thus a range of cell-type specific marker genes can be enriched in proportion to their expression in activated cells.

Some of the genes enriched in pS6 immunoprecipitates identify neural populations not previously known to be activated by salt challenge. Thus specific enrichment was detected for relaxin-1 (Rln1; 6.1-fold), a neuropeptide that can stimulate water intake (Thornton, S. M. & Fitzsimons, J. T. Journal of neuroendocrinology 7, 165-169 (1995); hereby incorporated by reference in its entirety) and can activate vasopressin/oxytocin neurons (Sunn, N. et al. Proc Natl Acad Sci USA 99, 1701-1706, doi:10.1073/pnas.022647699 (2002); hereby incorporated by reference in its entirety), but which has not previously been characterized in the hypothalamus due to its low expression level. Other enriched neuropeptides include urocortin-3 (Ucn3; 5.3-fold), which is related to Crh and expressed in a small population of neurons in the perifornical region, and somatostatin (Sst; 3.1-fold), which can promote vasopressin release (Brown, M. R., et al. Brain research 452, 212-218 (1988); hereby incorporated by reference in its entirety). It was found that some enriched genes, such as FosB (38-fold) and the chemokine Cxcl1 (13-fold), were not expressed at baseline but selectively induced in the activated neurons following salt challenge (FIG. 22e,f). Cxcl1 has been previously been shown to be upregulated in the PVN following osmotic stimulation (Koike, K. et al. Brain research. Molecular brain research 52, 326-329 (1997); hereby incorporated by reference in its entirety). These results were confirmed by microarray analysis, which identified four of these genes—Avp, Oxt, FosB, and Crh—as the four most highly enriched genes in the genome in pS6 immunoprecipitates from salt challenged animals relative to controls (FIG. 23b). A similar pattern of marker gene enrichment was observed by RNA sequencing (FIG. 23c). In contrast immunoprecipitation of total ribosomes from salt challenged animals enriched for none of these genes (FIG. 23d). The systematic identification of key genes expressed in osmoregulatory neurons validates the ability of this approach to identify ensembles of activated neurons.

TABLE 4
Taqman probes that recognize hypothalamic markers. Probes
include all neuropeptides encoded by the mouse genome that were detected by qPCR
in the hypothalamus. Additional probes were selected based on manual analysis in
situ hybridization data from the Allen Brain Atlas and GFP expression data from the
GENSAT projection in order to select genes that showed sparse, highly localized
expression within a specific anatomic region within the hypothalamus.

| GENE SYMBOL | GENE NAME | CLASS |
|---|---|---|
| PENK | Pro-enkephalin | Neuropeptide |
| POMC | Pro-opiomelanocortin | Neuropeptide |
| PDYN | Pro-dynorphin | Neuropeptide |
| PNOC | Prepro-nociceptin | Neuropeptide |
| AVP | Vasopressin | Neuropeptide |
| OXT | Oxytocin | Neuropeptide |
| GAST | Gastrin | Neuropeptide |
| CCK | Cholecystokinin | Neuropeptide |
| SST | Somatostatin | Neuropeptide |
| CORT | Cortistatin | Neuropeptide |
| NPVF | RF-amide related peptide, Neuorpeptide VF | Neuropeptide |
| NPFF | Neuropeptide FF | Neuropeptide |
| NPY | Neuropeptide Y | Neuropeptide |
| CALCA | Calcitonin 1, CGRP (calcitonin related polypeptide) | Neuropeptide |
| CALCB | Calcitonin 2 | Neuropeptide |
| IAPP | Amylin, Islet amyloid polypeptide | Neuropeptide |
| ADM | Adrenomedullin | Neuropeptide |
| NPPA | Atrial natriuretic factor | Neuropeptide |
| NPPC | Natriuretic peptide precursor C | Neuropeptide |
| GRP | Gastin releasing peptide | Neuropeptide |
| NMB | Neuromedin B | Neuropeptide |
| EDN3 | Endothelin 3 | Neuropeptide |
| SCT | Secretin | Neuropeptide |
| VIP | Vasoactive intestinal peptide | Neuropeptide |
| ADCYAP1 | Pituitary adneylcyclase-activated peptide | Neuropeptide |
| GHRH | Growth hormone releasing hormone | Neuropeptide |
| CRH | Corticotropin releasing hormone | Neuropeptide |
| UCN | Urocortin | Neuropeptide |
| UCN2 | Urocortin | Neuropeptide |
| UCN3 | Urocortin | Neuropeptide |
| TAC1 | Prepro-tachykinin A, substance P, | Neuropeptide |
| | Neurokinin A | |
| TAC2 | Aka Tac3; Prepro-tachykinin B, | Neuropeptide |
| | Neuromedin K, Neurokinin B | |
| NMS | Neuromedin S | Neuropeptide |
| NMU | Neuromedin U | Neuropeptide |
| AGT | Angiotensin | Neuropeptide |
| NTS | Neurotensin | Neuropeptide |
| CHGA | Chromogranin A | Neuropeptide |
| CHGB | Chromogranin B | Neuropeptide |
| SCG2 | Secretogranin II | Neuropeptide |
| SCG3 | Secretogranin III | Neuropeptide |
| SCG5 | SGNE1, Secretory granule | Neuropeptide |
| | neuroendocrine protein | |
| VGF | VGF nerve growth factor | Neuropeptide |
| GAL | Galanin | Neuropeptide |
| GALP | Galanin-like peptide | Neuropeptide |
| GnRH1 | Gonadotropin-releasing hormone 1 | Neuropeptide |
| NPB | Neuropeptide B | Neuropeptide |
| NPW | Neuropeptide W | Neuropeptide |
| NPS | Neuropeptide S | Neuropeptide |
| NXPH1 | Neurexophilin-1 | Neuropeptide |
| NXPH2 | Neurexophilin-2 | Neuropeptide |
| NXPH3 | Neurexophilin-3 | Neuropeptide |
| NXPH4 | Neurexophilin-4 | Neuropeptide |
| UTS2D | Urotensin-2-related peptide | Neuropeptide |
| RLN1 | Relaxin 1 | Neuropeptide |
| RLN3 | Relaxin 3 | Neuropeptide |
| TRH | Thyrotropin releasing hormone | Neuropeptide |
| PTHLH | Parathryroid hormone-like hormone | Neuropeptide |
| PMCH | Melanin concentrating hormone | Neuropeptide |
| HCRT | Hypocretin | Neuropeptide |
| CARTPT | Cocaine and amphetamine regulated | Neuropeptide |
| | transcript | |
| AGRP | Agouti related protein | Neuropeptide |
| APLN | Apelin | Neuropeptide |
| KISS1 | Kisspeptin, Metastasis-suppressor KiSS | Neuropeptide |
| DBI | Diazepam-binding inhibitor | Neuropeptide |
| CBLN1 | Cerebellin-1 | Neuropeptide |
| CBLN2 | Cerebellin-2 | Neuropeptide |
| CBLN4 | Cerebellin-4 | Neuropeptide |
| ADIPOQ | Adiponectin | Neuropeptide |
| RETN | Resistin | Neuropeptide |
| NUCB2 | Nucleobindin 2, Nesfatin | Neuropeptide |
| UBL5 | Ubiquitin-like 5 | Neuropeptide |
| SERPINA3K | serine (or cysteine) peptidase inhibitor, | Other |
| | clade A, member 3K | |
| NPY1R | Neuropeptide Y receptor 1 | Receptor |
| CITED1 | 00-interacting transactivator with | Transcription factor |
| | Glu/Asp-rich carboxy-terminal do | |
| ESYT3 | extended synaptotagmin-like protein 3 | Other |
| PRLR | Prolactin receptor | Receptor |
| ASB4 | ankyrin repeat and SOCS box- | Other |
| | containing 4 | |
| RGS9 | regulator of G-protein signaling 9 | Other |
| PLAGL1 | pleiomorphic adenoma gene-like 1 | Other |
| GABRE | gamma-aminobutyric acid (GABA) A | Receptor |
| | receptor, subunit epsilon | |
| TMEM176A | transmembrane protein 176A | Other |
| Ecel1 | Endothelin converting enzyme-like 1 | Other |
| PEG10 | paternally expressed 10 | Other |
| GRIK3 | glutamate receptor, ionotropic, kainate 3 | Receptor |
| Tbx3 | T-box 3 | Transcription factor |
| IRS4 | Insulin receptor substrate 4 | Other |
| TMED3 | transmembrane emp24 domain | Other |
| | containing 3 | |
| GPX3 | glutathione peroxidase 3 | Other |
| DLK1 | delta-like 1 homolog | Transcription factor |
| ARL10 | ADP-ribosylation factor-like 10 | Other |
| SPINT2 | serine protease inhibitor, Kunitz type 2 | Other |
| GPR165 | G protein-coupled receptor 165 | Receptor |
| Clcn5 | chloride channel 5 | Channel/Transporter |
| Celf6 | CUGBP, Elav-like family member 6 | Other |
| Rxfp3 | Relaxin family peptide receptor 3 | Receptor |
| Nnat | Neuronatin | Other |
| Mesdc2 | mesoderm development candidate 2 | Other |
| Slc2a1 | ut-1; Solute carrier family 2, facilitated | Channel/Transporter |
| | glucose transporter membe | |
| VAT1 | vesicle amine transport protein 1 | Channel/Transporter |
| | homolog (T californica) | |
| Adcyap1r1 | adenylate cyclase activating polypeptide | Receptor |
| | 1 receptor 1 | |
| Fezf1 | Fez family zinc finger 1 | Transcription factor |
| Slit3 | slit homolog 3 (Drosophila) | Other |
| Gda | guanine deaminase | Other |
| Rreb1 | ras responsive element binding protein 1 | Transcription factor |
| AMIGO2 | adhesion molecule with Ig like domain 2 | Other |
| Doc2b | double C2, beta | Other |
| Pvrl3 | poliovirus receptor-related 3 | Other |
| Icam5 | intercellular adhesion molecule 5, | Other |
| | telencephalin | |
| Glra1 | glycine receptor, alpha 1 subunit | Receptor |
| Chrm5 | cholinergic receptor, muscarinic 5 | Receptor |
| Camk1g | calcium/calmodulin-dependent protein | Other |
| | kinase I gamma | |
| Itpr1 | inositol 1,4,5-triphosphate receptor 1 | Other |
| Lmo3 | LIM domain only 3 | Transcription factor |
| Cacna2d1 | calcium channel, voltage-dependent, | Channel/Transporter |
| | alpha2/delta subunit 1 | |
| Kcnab1 | ssium voltage-gated channel, shaker- | Channel/Transporter |
| | related subfamily, beta mem | |
| Syt10 | synaptotagmin 10 | Other |
| Lhx1 | LIM homeobox protein 1 | Transcription factor |
| Vipr2 | VIP receptor 2 | Receptor |
| Rasl11b | Ras like 11b | Other |
| Rgs16 | Regulator of G-protein signaling 16 | Other |
| Rorb | RAR-related orphan receptor beta | Transcription factor |
| Prokr2 | prokineticin receptor 2 | Receptor |
| Rora | RAR-related orphan receptor alpha | Transcription factor |
| NR1D1 | nuclear receptor subfamily 1, group D, | Transcription factor |
| | member 1 | |
| Zim1 | zinc finger, imprinted 1 | Transcription factor |
| Flrt3 | fibronectin leucine rich transmembrane | Other |
| | protein 3 | |
| Zic1 | zinc finger protein of the cerebellum 1 | Transcription factor |
| Slc2a13 | solute carrier family 2 (facilitated | Channel/Transporter |
| | glucose transporter), member 13 | |
| Npsr1 | Neuropeptide S receptor 1 | Receptor |
| Fezf2 | Fez family zinc finger 2 | Transcription factor |
| Tacr3 | Tachykinin receptor 3 | Receptor |
| Ly6H | Lymphocyte antigen 6 complex, locus H | Other |
| Ntsr1 | Neurotensin receptor 1 | Receptor |
| Pitx2 | Paired-like homeodomain transcription | Transcription factor |
| | factor 2 | |
| Gabrq | Gamma-aminobutyric acid (GABA) A | Receptor |
| | receptor, subunit theta | |
| Calcr | Calcitonin receptor | Receptor |
| GPR101 | GPCR 101 | Receptor |
| Pou6f2 | POU domain, class 6, transcription | Transcription factor |
| | factor 2 | |
| Crhr2 | Corticotropin releasing hormone | Receptor |
| | receptor 2 | |
| Htr1a | 5-hydroxytryptamine (serotonin) | Receptor |
| | receptor 1A | |
| Htr1b | 5-hydroxytryptamine (serotonin) | Receptor |
| | receptor 1B | |
| Htr2a | 5-hydroxytryptamine (serotonin) | Receptor |
| | receptor 2A | |
| Htr2c | 5-hydroxytryptamine (serotonin) | Receptor |
| | receptor 2C | |
| Htr3b | 5-hydroxytryptamine (serotonin) | Receptor |
| | receptor 3B | |
| Htr4 | 5-hydroxytryptamine (serotonin) | Receptor |
| | receptor 4 | |
| Htr5A | 5-hydroxytryptamine (serotonin) | Receptor |
| | receptor 5A | |
| Htr6 | 5-hydroxytryptamine (serotonin) | Receptor |
| | receptor 6 | |
| Zfhx4 | zinc finger homeodomain 4 | Transcription factor |
| Ar | Androgen receptor | Transcription factor |
| Trhr | Thyrotropin releasing hormone receptor | Receptor |
| Cnr1 | Cannabinoid receptor 1 | Receptor |
| MC4R | Melanocortin 4-receptor | Receptor |
| NPY5R | Neuropeptide Y receptor 5 | Receptor |
| NPY2R | Neuropeptide Y receptor 2 | Receptor |
| PGR | progesterone receptor | Transcription factor |
| OXTR | oxytocin receptor | Receptor |
| Gpr83 | G protein-coupled receptor 83 | Receptor |
| Pcsk1 | Proprotein convertase subtilisin/kexin | Other |
| | type 1 | |
| lhx9 | Lim homeobox protein 9 | Transcription factor |
| agtr1a | angiotensin II receptor 1a | Receptor |
| Sim1 | single minded 1 | Transcription factor |
| Gsbs | G substrate | Other |
| Calb1 | calbindin 1 | Other |
| Calb2 | calbindin 2 | Other |
| Chrna3 | cholinergic receptor, nicotinic, alpha | Receptor |
| | polypeptide 3 | |
| Chrna4 | cholinergic receptor, nicotinic, alpha | Receptor |
| | polypeptide 4 | |
| Chrna7 | cholinergic receptor, nicotinic, alpha | Receptor |
| | polypeptide 7 (Chrna7 | |
| Avpr1a | arginine vasopressin receptor 1A | Receptor |
| GBX2 | gastrulation brain homeobox 2 | Transcription factor |
| DDC | dopa decarboxylase | Other |
| SYTL4 | synaptotagmin-like 4 | Other |
| NGB | neuroglobin | Other |
| NHLH2 | nescient helix loop helix 2 | Transcription factor |
| nkx2-1 | NK2 homeobox 1 | Transcription factor |
| isl1 | ISL1 transcription factor | Transcription factor |
| BRS3 | bombesin-like receptor 3 | Receptor |
| Slc18a2 | vesicular monamine transporter | Channel/Transporter |
| NR5A1 | SF1 | Transcription factor |
| P2RY1 | purinergic receptor P2Y, P2Y | Channel/Transporter |
| Esr1 | estrogen receptor alpha | Transcription factor |
| rp127 | ribosomal protein L27 | Other |
| rp123 | ribosomal protein L23 | Other |
| actb | Actin | Other |
| Syt1 | synaptotagmin 1 | Other |
| slc1a2 | glutamate transproter in glia | Channel/Transporter |
| nefl | neurofilament, light | Other |
| slc12a5 | KCC2, neuron specific potassium | Channel/Transporter |
| | symporter | |
| snap25 | synaptosomal associated protein 25 | Other |
| gfap | glial fibrillary acidic protein | Other |
| HDC | Histidine decarboxylase | Other |
| Ache | Acetylcholinesterase | Other |
| Mal | myelin and lymphocyte protein, | Other |
| | oligodendrocyte marker | |
| FA2H | fatty acid 2-hydroxylase, | Other |
| | oligodendrocyte marker | |
| Slc6a3 | Dopamine Transporter; dopamine | Channel/Transporter |
| | marker | |
| TH | Tyrosine hydroxylase; dopamine marker | Other |
| GAD2 | glutamic acid decarboxylase 2 | Other |
| GAD1 | GAD67, glutamic acid decarboxylase 1 | Other |
| NOS1 | nitric oxide synthase 1, neuronal | Other |
| Fxyd6 | FXYD domain-containing ion transport | Other |
| | regulator 6 | |
| hap1 | huntingtin-associated protein 1 | Other |
| Slc17a7 | Vglut1; solute carrier family 17 member 6 | Channel/Transporter |
| Slc17a6 | Vglut2; solute carrier family 17 member 6 | Channel/Transporter |
| Slc1a1 | EAAT3, neuronal/epithelial high affinity | Channel/Transporter |
| | glutamate transporter | |
| Sgsm1 | small G protein signaling modulator 1 | Other |
| Susd2 | sushi domain containing 2 | Other |
| Pcsk1n | Neuropeptide, proSAAS | Neuropeptide |
| Ghsr | Growth hormone secretagogue receptor | Receptor |
| Npr3 | natriuretic peptide receptor 3 (NPR-C) | Receptor |
| Crabp1 | cellular retinoic acid binding protein | Transcription factor |
| Scn9a | sodium channel, voltage-gated, type IX, | Channel/Transporter |
| | alpha | |
| Scn7a | sodium channel, voltage-gated, type VII, | Channel/Transporter |
| | alpha | |
| kcnk2 | potassium channel subfamily K member 2 | Channel/Transporter |
| Adra2a | alpha 2A adrenergic receptor | Receptor |
| Per1 | Period homolog 1 | Transcription factor |
| Per2 | Period homolog 1 | Transcription factor |
| Drd2 | Dopamine receptor 2 | Receptor |
| GPR50 | G protein coupled receptor 50 | Receptor |
| Drd1a | Dopamine receptor 1a | Receptor |
| Aplnr | Apelin Receptor | Receptor |
| Fzd5 | frizzled homolog 5 | Transcription factor |
| Pou2f2 | POU domain, class 2, transcription | Transcription factor |
| | factor 2 | |
| Sox3 | SRY (sex determining region Y)-box 3 | Transcription factor |
| Six3 | sine oculis-related homeobox 3 homolog | Transcription factor |
| Qrfpr | pyroglutamylated RFamide peptide | Receptor |
| | receptor | |
| Oprl1 | opiod receptor like 1 | Receptor |
| Gck | glucokinase | Other |
| Esr2 | estrogen receptor beta | Transcription factor |
| MC3R | melanocortin 3-receptor | Receptor |
| Fos | FBJ osteosarcoma oncogene | Transcription factor |
| FosB | FBJ murine osteosarcoma viral | Transcription factor |
| | oncogene homolog B | |
| Egr1 | early growth response 1; NGFI-A | Transcription factor |
| Egr4 | early growth response 4 | Transcription factor |
| Nr4a1 | Nur77; NGFI-B, immediate early gene | Transcription factor |
| Arc | activity-regulated cytoskeleton- | Transcription factor |
| | associated protein; Arg3.1 | |

TABLE 5
Summary of Taqman array data. Data are presented as the mean
differential fold-enrichment (IP/input)stimulus/(IP/Input)control. The
number of independent experiments for stimulus and control for each
condition are listed in the first row.

Δ fold-enrichment (stimulus/control)

| | | | Sched. | |
|---|---|---|---|---|
| | Osmotic | Ghrelin | Feeding | Fasting |
| N (Stimulus, | (5, 6) | (4, 6) | (4, 6) | (3, 2) |
| Control) | | | | |
| Ach3 | 1.471 | 0.955 | 0.561 | 1.686 |
| actb | 1.119 | 0.696 | 0.661 | 1.077 |
| Adcyap1 | 1.766 | 0.951 | 0.946 | 1.398 |
| Adcyap1r1 | 1.542 | 0.923 | 1.39 | 0.739 |
| Adra2a | 1.081 | 1.215 | 1.494 | 1.962 |
| AgRP | 1.041 | 27.498 | 9.249 | 9.68 |
| Agt | 0.53 | 2.137 | 2.053 | 0.38 |
| Agtr1a | 3.681 | 4.599 | ND | ND |
| Amigo2 | 1.932 | 1.235 | 1.951 | 1.357 |
| Apln | 0.459 | 1.292 | 1.764 | 0.247 |
| Ar | 1.581 | 1.862 | 1.074 | 1.022 |
| Arc | 2.928 | ND | ND | ND |
| Arl10 | 1.486 | 0.74 | 0.56 | 1.289 |
| Asb4 | 0.84 | 1.089 | 1.264 | 2.044 |
| Avp | 48.772 | 1.281 | 1.214 | 0.92 |
| Avpr1a | 1.018 | 1.116 | 0.69 | 1.049 |
| Brs3 | 0.96 | 0.652 | 2.323 | 1.33 |
| Cacna2d1 | 2.526 | 1.657 | 1.13 | 0.96 |
| Calb1 | 1.554 | 0.988 | 1 | 1.154 |
| Calb2 | 1.025 | 1.041 | 0.862 | 1.218 |
| Calca | 1.043 | 1.34 | 1.704 | 1.283 |
| Calcr | 3.898 | 4.296 | 2.585 | 1.251 |
| Camk1g | 1.135 | 1.657 | 0.724 | 1.103 |
| Caprin2 | 0.926 | 0.703 | 1.63 | 2.822 |
| Cartpt | 1.173 | 0.93 | 0.967 | 1.615 |
| Cbln1 | 1.43 | 0.786 | 0.506 | 1.055 |
| Cbln2 | 1.212 | 0.754 | 0.719 | 0.812 |
| Cbln4 | 1.189 | 1.208 | 0.837 | 1.135 |
| Celf6 | 2.792 | 1.44 | 1.046 | 1.349 |
| c-fos | 1.522 | ND | NE | ND |
| CHGA | 1.12 | 0.879 | 1.042 | 1.874 |
| Chgb | 0.824 | 0.744 | 0.899 | 1.757 |
| Chrm5 | 0.84 | 0.793 | 1.04 | 0.867 |
| Chrna3 | 0.87 | 2.23 | 1.183 | 1.802 |
| Chrna4 | 0.73 | 1.406 | 1.321 | 1.24 |
| Chrna7 | 1.713 | 1.301 | 1.027 | 0.89 |
| Cited1 | 1.29 | 2.722 | 1.932 | 1.889 |
| Clcn5 | 1.294 | 0.483 | 1.634 | 1.214 |
| Cnr1 | 1.72 | 1.529 | 0.696 | 1.293 |
| Crabp1 | 0.532 | 0.849 | 0.93 | 1.098 |
| Crh | 10.119 | 3.721 | 3.144 | ND |
| Crhr2 | 2.275 | 1.457 | 2.635 | 3.302 |
| Cxcl1 | 12.835 | ND | ND | ND |
| DBI | 0.72 | 1.447 | 1.44 | 0.382 |
| Ddc | 1.09 | 1.258 | 1.101 | 1.155 |
| Dlk1 | 1.873 | 1.594 | 1.397 | 1.917 |
| Doc2b | 2.317 | 1.41 | 1.248 | 0.711 |
| Drd1a | 0.635 | 1.831 | 1.165 | 1.052 |
| Drd2 | 0.946 | 1.333 | 1.058 | 0.976 |
| Ecel1 | 1.742 | 2.444 | 2.492 | 1.724 |
| Egr1 | 2.511 | ND | ND | ND |
| Egr4 | 7.557 | ND | ND | ND |
| Esr1 | 1.582 | 1.294 | 0.965 | 0.963 |
| Esr2 | ND | ND | ND | 1.129 |
| Esyt3 | 2.639 | 2.392 | 3.247 | 1.939 |
| Fezf1 | 2.526 | 3.125 | 1.037 | 0.762 |
| Fezf2 | 5.539 | 1.879 | 2.986 | 2.15 |
| Flrt3 | 1.44 | 1.347 | 1.653 | 1.813 |
| FosB | 38.767 | ND | ND | ND |
| Fxyd6 | 1.006 | 1.606 | 0.899 | 1.309 |
| Fzd5 | 0.316 | 0.97 | 0.4 | 1.176 |
| Gabre | 1.243 | 1.238 | 2.015 | 1.958 |
| Gabrq | 2.207 | 1.412 | 0.945 | 1.811 |
| GAD1 | 0.69 | 1.209 | 0.87 | 1.6 |
| GAD2 | 0.554 | 0.788 | 0.744 | 0.749 |
| Gal | 4.614 | 3.632 | 4 | 8.293 |
| Gast | 2.554 | 2.527 | 2.794 | 1.811 |
| Gbx2 | 0.64 | 0.765 | 0.878 | 2.112 |
| Gck | 1.68 | 3.046 | 2.513 | 1.284 |
| Gda | 3.082 | 1.777 | 1.435 | 1.404 |
| Gfap | 1.469 | 0.951 | 2.267 | 0.727 |
| Ghrh | 1.115 | 2.554 | 2.95 | 1.341 |
| Ghsr | 1.829 | 7.098 | 6.818 | 4.589 |
| Glra1 | 1.104 | 1.304 | 1 | 2.217 |
| Gnrh1 | 0.98 | 1.167 | 1.361 | 0.436 |
| Gpr101 | 1.285 | 1.699 | 0.87 | 2.308 |
| Gpr165 | 2.096 | 1.091 | 0.882 | 2.098 |
| GPR50 | 3.225 | 2.696 | 7.117 | 1.282 |
| Gpr83 | 1.579 | 1.415 | 1.188 | 0.492 |
| Gpx3 | 2.29 | 1.003 | 1.235 | 1.121 |
| Grik3 | 1.942 | 1.019 | 1.36 | ND |
| Grp | 0.912 | 1.433 | 1.668 | 1.122 |
| Gsbs | 2.776 | 1.723 | 4.76 | 0.857 |
| hap1 | 0.954 | 1.25 | 0.931 | 1.072 |
| hba-a1 | 0.379 | 0.962 | 1.612 | 0.869 |
| hbb-b1 | 0.32 | 0.801 | 1.724 | 0.854 |
| Hcrt | 1.066 | 2.132 | 1.524 | 1.691 |
| Hdc | 0.925 | 0.847 | 2.245 | 0.895 |
| Htr1a | 0.767 | 1.154 | 1.038 | 0.775 |
| Htr1b | 0.923 | 1.737 | 0.589 | 1.411 |
| Htr2a | 1.27 | 1.002 | 2.38 | 1.413 |
| Htr2c | 0.929 | 0.82 | 0.966 | 1.636 |
| Htr4 | 0.625 | ND | ND | 0.892 |
| Htr5a | 0.787 | 1.78 | 1.366 | 2.507 |
| Htr6 | 0.82 | 0.725 | 1.299 | 0.794 |
| Icam5 | 1.086 | 1.305 | 0.752 | 0.995 |
| Irs4 | 1.864 | 1.972 | 1.694 | 1.396 |
| Isl1 | 2.529 | 2.099 | 2.687 | 1.226 |
| Itpr1 | 2.239 | 1.474 | 2.065 | 1.108 |
| Kcnab1 | 2 | 2.379 | 1.292 | 0.995 |
| kcnk2 | 0.955 | 1.116 | 1.073 | 0.954 |
| Kiss1 | 1.667 | 1.223 | ND | 1.636 |
| Lhx1 | 1 | 1.211 | 0.766 | 1.515 |
| Lhx9 | 1.428 | 1.401 | 1.57 | 0.996 |
| Lmo3 | 1.624 | 1.157 | 0.856 | 0.885 |
| Ly6H | 1.564 | 1.655 | 1.263 | 1.444 |
| Mal | 1.166 | 0.843 | 1.429 | 1.65 |
| MC3R | 0.367 | 0.637 | 0.575 | 0.883 |
| Mc4R | 1.004 | 0.691 | 0.998 | 1.353 |
| Mesdc2 | 1.805 | 0.941 | 0.715 | 0.988 |
| Mpzl2 | 3.502 | ND | ND | ND |
| Nefl | 1.233 | 1.134 | 1.284 | 1.288 |
| Ngb | 1.316 | 1.714 | 1.946 | 1.34 |
| Nhlh2 | 7.281 | 1.739 | 3.378 | 0.906 |
| Nkx2-1 | 1.481 | 0.372 | 0.782 | 0.61 |
| Nmb | 0.605 | 0.17 | 0.83 | 0.405 |
| Nms | 0.631 | 0.481 | 0.962 | 2.464 |
| Nnat | 1.748 | 0.818 | 0.823 | 1.01 |
| Nos1 | 2.488 | 2.555 | 3.425 | 0.556 |
| Npb | 0.871 | 1.592 | 1.189 | 3.122 |
| Npff | 7.071 | 3.411 | ND | 1.719 |
| Nppa | 4.972 | 6.244 | ND | ND |
| Nppc | 0.788 | 0.877 | 1.019 | 2.379 |
| Npr3 | 0.702 | 0.896 | 1.327 | 1.312 |
| Npsr1 | 1.168 | 0.779 | ND | 1.589 |
| Npvf | 0.614 | 1.449 | 7.273 | 0.603 |
| NPY | 1.044 | 24.48 | 17.72 | 15.207 |
| Npy1r | 1.223 | 1.454 | 1.886 | 0.907 |
| Npy2r | 2.095 | 1.732 | 2.383 | 1.873 |
| Npy5R | 1.548 | ND | ND | 1.124 |
| NR1D1 | 0.746 | 1.219 | 0.709 | 0.702 |
| Nr4a1 | 6.355 | ND | ND | ND |
| Nts | 1.628 | 2.386 | 1.467 | 2.428 |
| Ntsr1 | 1.709 | 1.356 | 1.532 | 1.365 |
| Nucb2 | 1.54 | 0.89 | 0.69 | 1.453 |
| Nupr1 | 2.131 | ND | ND | ND |
| Nxph1 | 1.37 | 0.749 | 0.922 | 1.585 |
| Nxph3 | 0.689 | 0.506 | 0.722 | 1.475 |
| Nxph4 | 1.604 | 1.554 | 0.352 | 1.399 |
| Oprl1 | 0.896 | 1.791 | 1.254 | 1.409 |
| Oxt | 13.988 | 1.628 | 1.615 | 1.332 |
| OxtR | 0.684 | 1.095 | 1.115 | 0.502 |
| P2RY1 | 0.879 | 0.855 | 3.091 | 1.938 |
| Pcsk1 | 3.291 | 1.884 | 1.653 | 1.003 |
| Pcsk1n | 0.645 | 0.934 | 0.484 | 0.871 |
| Pdyn | 3.44 | 1.092 | 2.942 | 1.839 |
| Peg10 | 2.2 | 0.79 | 0.919 | 1.875 |
| Penk | ND | 1.309 | 0.304 | 1.374 |
| Per1 | 1.148 | 0.908 | 1.324 | 1.127 |
| Per2 | 1.248 | 1.631 | 0.736 | 1.037 |
| Pgr | 0.898 | 1.409 | 0.883 | 1.364 |
| Pitx2 | 2.319 | 1.569 | 1.488 | 1.009 |
| Plagl1 | 1.858 | 1.572 | 1.324 | 1.323 |
| PMCH | 0.287 | 0.485 | 0.25 | 2.113 |
| Pnoc | 1.965 | 1.838 | 2.468 | 1.145 |
| POMC | 0.709 | 0.917 | 0.663 | 0.11 |
| Pou2f2 | 0.571 | 1.306 | 0.77 | 1.083 |
| Pou6f2 | 0.318 | 0.469 | 0.322 | 0.74 |
| Prlr | 1.278 | 1.486 | 1.304 | 0.49 |
| Prokr2 | 2.23 | 1.59 | 1.588 | 2.406 |
| Pthlh | 0.957 | 0.783 | 0.656 | 1.74 |
| Pvrl3 | 1.434 | 0.78 | 1.358 | 1.997 |
| Qrfpr | 1.033 | 0.734 | 0.458 | 1.56 |
| Rgs16 | 0.787 | 1.039 | 0.939 | 1.055 |
| Rgs9 | 1.102 | 1.171 | 2.045 | 1.159 |
| Rln1 | 6.07 | 0.33 | 1.082 | 1.44 |
| Rora | 0.849 | 1.479 | 1.399 | 1.175 |
| Rorb | 1.382 | 0.948 | 1.506 | 1.53 |
| rpl23 | 1.246 | 1.022 | 0.77 | 1.095 |
| rpL27 | 1.054 | 1.005 | 1.507 | 1.285 |
| rpl27 | 1.035 | 0.938 | 1.198 | 1.232 |
| rpL27 | 1.041 | 1.132 | 1.238 | 1.078 |
| rpL27 | 0.918 | 0.902 | 0.908 | 1.052 |
| rpL27 | 0.762 | 0.97 | 0.945 | 0.976 |
| rpL27 | 1.187 | 1.081 | 0.923 | 0.947 |
| rpL27 | 1.038 | 0.79 | 0.662 | 0.9 |
| rpl27 | 1.018 | 1.221 | 0.823 | 0.884 |
| Rreb1 | 1.128 | 1.107 | 2.031 | 0.663 |
| Rxfp3 | 3.254 | 1.667 | 1.393 | 2.302 |
| Scg2 | 1.864 | 1.205 | 0.879 | 2.329 |
| Scg3 | 1.997 | 1.625 | 1.589 | 1.213 |
| SCG5 | 1.991 | 1.853 | 0.963 | 1.602 |
| Scn7a | ND | ND | ND | 1.565 |
| Scn9a | 1.769 | 1.018 | 2.506 | 1.79 |
| SF1 | 0.397 | 0.5 | 0.952 | 0.964 |
| Sgsm1 | 0.883 | 1.227 | 0.925 | 1.193 |
| Sim1 | 4.041 | 2.114 | 1.424 | 0.464 |
| Six3 | 0.521 | 1.004 | 0.831 | 0.534 |
| slc12a5 | 1.336 | 0.659 | 0.662 | 1.323 |
| Slc17a6 | 0.745 | 0.975 | 0.846 | 1.06 |
| Slc17a7 | 0.826 | 1.444 | 1.596 | 2.604 |
| Slc18a2 | 1.016 | 0.888 | 1.28 | 2.109 |
| Slc1a1 | 0.523 | 1.158 | 1.014 | 0.832 |
| Slc1a2 | 1.021 | 1.114 | 2.207 | 0.455 |
| Slc2a1 | 2.784 | 2.771 | 3.415 | 1.182 |
| Slc2a13 | 2.007 | 0.941 | 0.713 | 0.852 |
| Slc6a3 | 0.451 | 0.461 | 0.832 | 1.088 |
| Slit3 | 1.368 | 0.903 | 1.49 | 1.986 |
| snap25 | 1.272 | 0.829 | 0.831 | 1.139 |
| Sox3 | 0.712 | 0.505 | 0.491 | 0.663 |
| Spint2 | 2.947 | 0.921 | 0.912 | 1.043 |
| SST | 3.129 | 1.229 | 0.517 | 1.367 |
| Susd2 | 0.666 | 1.003 | 0.908 | 0.764 |
| Syt1 | 1.178 | 1.024 | 1.177 | 1.142 |
| Syt10 | 1.017 | 1.664 | 1.055 | 0.705 |
| Tac1 | 1.895 | 1.347 | 2.273 | 1.662 |
| Tac2 | 0.775 | 0.69 | 0.942 | 1.59 |
| Tacr3 | 1.031 | 0.605 | 1.462 | 1.795 |
| Tbx3 | 2.39 | 4.749 | 4.641 | 0.71 |
| TH | 1.226 | 1.595 | 1.111 | 1.354 |
| Tmed3 | 2.282 | 1.059 | 1.002 | 1.328 |
| Tmem176A | 1.971 | 1.328 | 1.374 | 1.457 |
| Trh | 1.264 | 1.188 | 0.741 | 0.958 |
| Trhr | 1.174 | 1.179 | 1.376 | 1.633 |
| Ubl5 | 1.075 | 1.051 | 1.014 | 1.298 |
| UCN3 | 5.324 | 2.778 | 2.44 | 1.207 |
| Vat1 | 1.577 | 1.08 | 1.189 | 1.541 |
| Vgf | 2.563 | 1.606 | 1.159 | 3.085 |
| Vip | 0.995 | 1.367 | 0.977 | 1.295 |
| Vipr2 | 0.793 | 0.834 | 0.464 | 4.016 |
| Zfhx4 | 2.33 | 2.619 | 2.809 | 0.789 |
| Zic1 | 1.641 | 1.153 | 1.201 | 1.231 |
| Zim1 | 1.167 | 0.776 | 1.808 | 1.039 |

### The Hypothalamic Response to Fasting

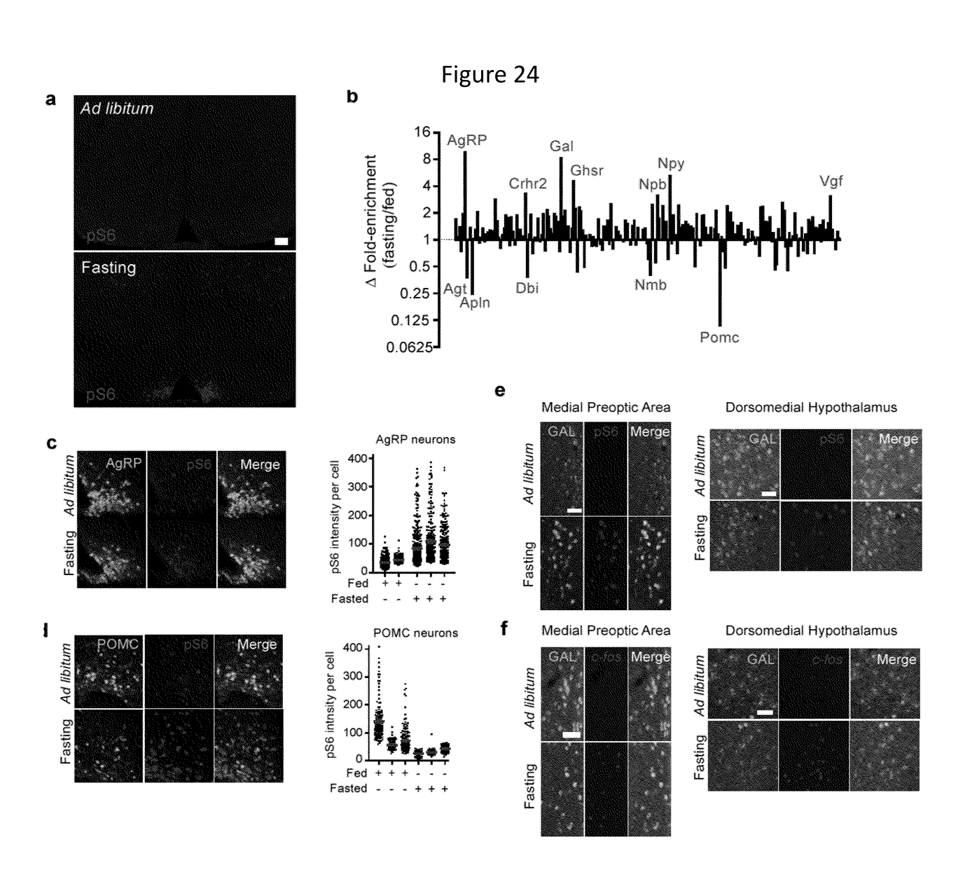

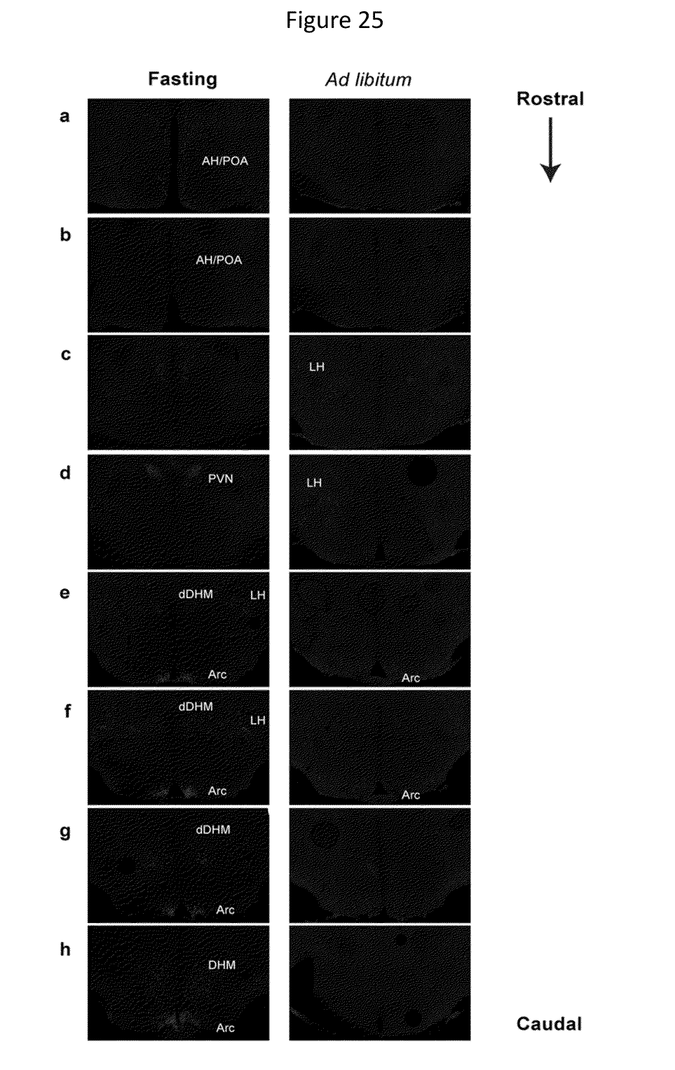

A different set of neurons in the hypothalamus regulate food intake and coordinate the response to food restriction. To identify components of this system, mice were exposed to a series of nutritional perturbations, beginning with fasting. Mice were fasted overnight, sacrificed at the beginning of the light phase, and the extent of S6 ribosome phosphorylation was assayed by immunostaining. It was found that fasting induced strong pS6 in the arcuate nucleus of the hypothalamus as well as in the dorsomedial hypothalamus (DMH) and scattered cells of the medial preoptic area (MPA; FIG. 24a and FIG. 25). To identify fasting-regulated neurons in each of these regions, phosphorylated ribosomes were immunoprecipitated from hypothalamic homogenates of fasted and fed animals and analyzed the enrichment of cell-type specific RNAs.

Markers for many cell-types that are known to regulate feeding were enriched. Thus two of the most enriched transcripts in response to fasting were AgRP and NPY (FIG. 24b). These two neuropeptides can be co-expressed in critical neurons of the arcuate nucleus that promote food intake (Elmquist, J. K., et al. The Journal of comparative neurology 493, 63-71, doi:10.1002/cne.20786 (2005); hereby incorporated by reference in its entirety), and immunostaining confirmed that fasting induces a selective increase in pS6 in these cells (FIG. 24c; Villanueva, E. C. et al. Endocrinology 150, 4541-4551, doi:10.1210/en.2009-0642 (2009), hereby incoporated by reference in its entirety). It was also observed enrichment for genes such as the ghrelin receptor (Ghsr), which can be expressed in most AgRP/NPY neurons (Willesen, M. G., et al. Neuroendocrinology 70, 306-316 (1999), hereby incorporated by reference in its entirety) and the neuropeptide VGF, which can be induced in AgRP neurons following fasting (Hahm, S. et al. The Journal of neuroscience 22, 6929-6938, doi:20026687 (2002), hereby incorporated by reference in its entirety). Other enriched genes, such as the neuropeptides NPB and MCH, can delineate additional distinct populations of neurons that have been reported to promote feeding (FIG. 24b; Dun, S. L. et al. Brain research 1045, 157-163, doi:10.1016/j.brainres.2005.03.024 (2005), hereby incoporated by reference in its entirety).

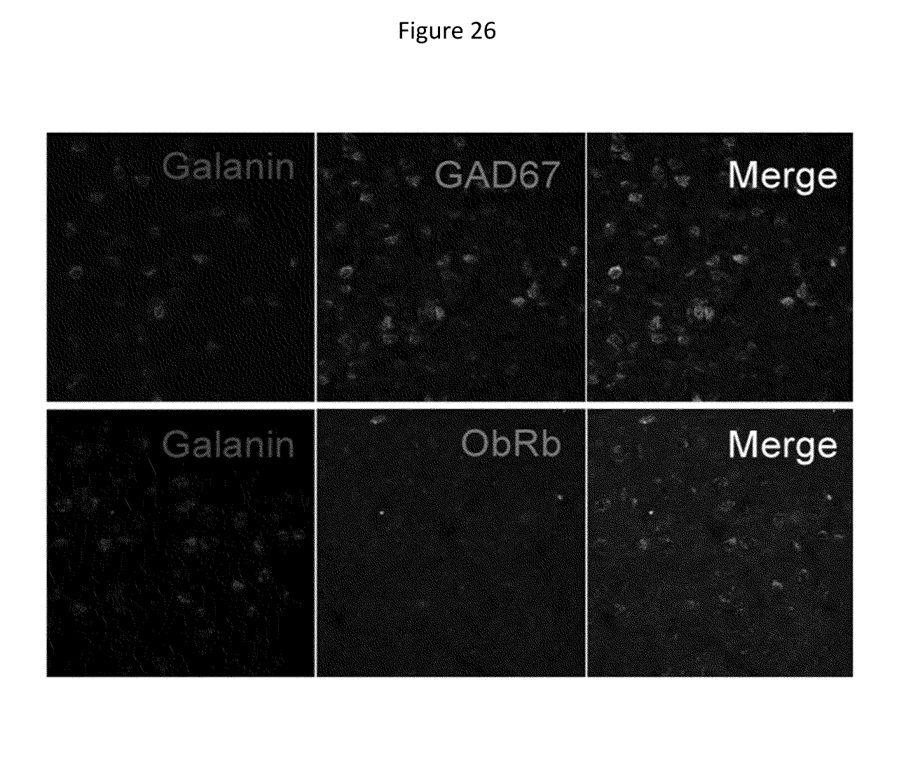

Galanin was one of the most strongly enriched genes in pS6 immunoprecipitates from fasted animals (8.3-fold, FIG. 24b). Galanin can stimulate feeding when injected directly into the hypothalamus (Parker, J. A. & Bloom, S. R. Neuropharmacology, doi:10.1016/j.neuropharm.2012.02.004 (2012); hereby incoporated by reference in its entirety), but the regulation of galanin neurons by changes in nutritional state has not been described and the role of galanin expressing neurons in the response to food restriction has been nebulous. It was found that fasting induced a marked increase in ribosome phosphorylation in a specific subset of galanin neurons located in the DMH and MPA (FIG. 24e), but not the Arc or SON. Galanin neurons in these two regions also expressed c-fos after an overnight fast (FIG. 24f), confirming that they can be activated by food restriction. Unlike AgRP neurons, which can be concentrated in the Arc, galanin neurons can be dispersed throughout multiple hypothalamic regions. As a result these neurons can be difficult to identify visually but nonetheless were revealed directly by capturing RNA from activated cells. The neurochemical identity of these cells were further characterized, showing that the majority of galanin neurons in the DMH were positive for GAD67, indicating that they produce the inhibitory neurotransmitter GABA, but were negative for the leptin receptor, indicating that they do not directly sense changes in plasma leptin (FIG. 26). Thus galanin neurons in the DMH and MPA represent a new population of fasting activated cells in the hypothalamus (as shown by increased c-fos expression) with a localization and regulation distinct from AgRP neurons.

As all neurons can have a basal level of ribosome phosphorylation, it was expected that neural inhibition might result in a decrease in pS6, which would be detected as the depletion of transcripts from pS6 immunoprecipitates. Consistent with this, it was found that the neuropeptide POMC was the most depleted transcript in response to fasting (9.2-fold; FIG. 24b). POMC is expressed in a population of neurons in the Arc that can inhibit food intake and are downregulated by fasting (Elmquist, J. K., et al. The Journal of comparative neurology 493, 63-71, doi:10.1002/cne.20786 (2005); hereby incorporated by reference in its entirety). Although fasting can increase the level of pS6 in the Arc overall (largely as a result of AgRP neuron activation, FIG. 24a), it was observed by quantitative imaging that fasting decreases the density of pS6 specifically within POMC cells (FIG. 24d). This demonstrates that pS6 ribosome profiling can also reveal markers for neurons that are inhibited. In addition to POMC, depletion of several additional neuropeptides that have been reported to inhibit feeding, including apelin (which is co-expressed with POMC), angiotensin, diazopam-binding inhibitor, and neuromedin B was observed (Reaux-Le Goazigo, A. et al. Am J Physiol Endocrinol Metab 301, E955-966, doi:10.1152/ajpendo.00090.2011 (2011); Porter, J. P. & Potratz, K. R. Am J Physiol Regul Integr Comp Physiol 287, R422-428, doi:10.1152/ajpregu.00537.2003 (2004); de Mateos-Verchere, J. G., et al. European journal of pharmacology 414, 225-231 (2001); and Merali, Z., et al. Neuropeptides 33, 376-386, doi:10.1054/npep.1999.0054 (1999); each of which is incoporated by reference in its entirety), suggesting that each of these peptides resides in a population of fasting-inhibited cells (FIG. 24b). Note that the depletion of these peptides is not a result of changes in their expression level, as only the ratio of RNA was analyze in the immunoprecipitate versus the tissue as a whole (IP/input). Rather experiments were performed to enrich or deplete for RNA from neurons based on whether the state of activation of that neuron has changed. The ability to detect neural inhibition by ribosome profiling contrasts with c-fos staining, which can have a limited ability to detect downregulation due to the low level of c-fos expression in most cells at baseline.

### Scheduled Feeding Induces pS6 in the Arc and DMH

While fasting can reveal the response to chronic energy deficit, most human feeding takes place during meals that occur at regular times in the day.

Rodents allowed daily access to food only during a scheduled window can synchronize their metabolism and activity to the time of food availability (Mistlberger, R. E. Physiology & behavior 104, 535-545, doi:10.1016/j.physbeh.2011.04.015 (2011); hereby incorporated by reference in its entirety). This behavioral adaptation can be characterized by a burst of locomoter activity just prior to food presentation known as food-anticipatory activity (FAA), and this process can be associated with the activation of neurons in multiple hypothalamic regions, including the DMH and Arc. Despite extensive investigation into the mechanism of FAA, the identity of the activated cell-types and their specific roles, in particular those in the DMH, are largely unknown. Thus experiments were designed to identify neurons with a specialized function associated with scheduled feeding. Unlike fasting, scheduled feeding can allow for more precise synchronization of behavior, enabling for detailed analysis of temporal changes in cell activation.

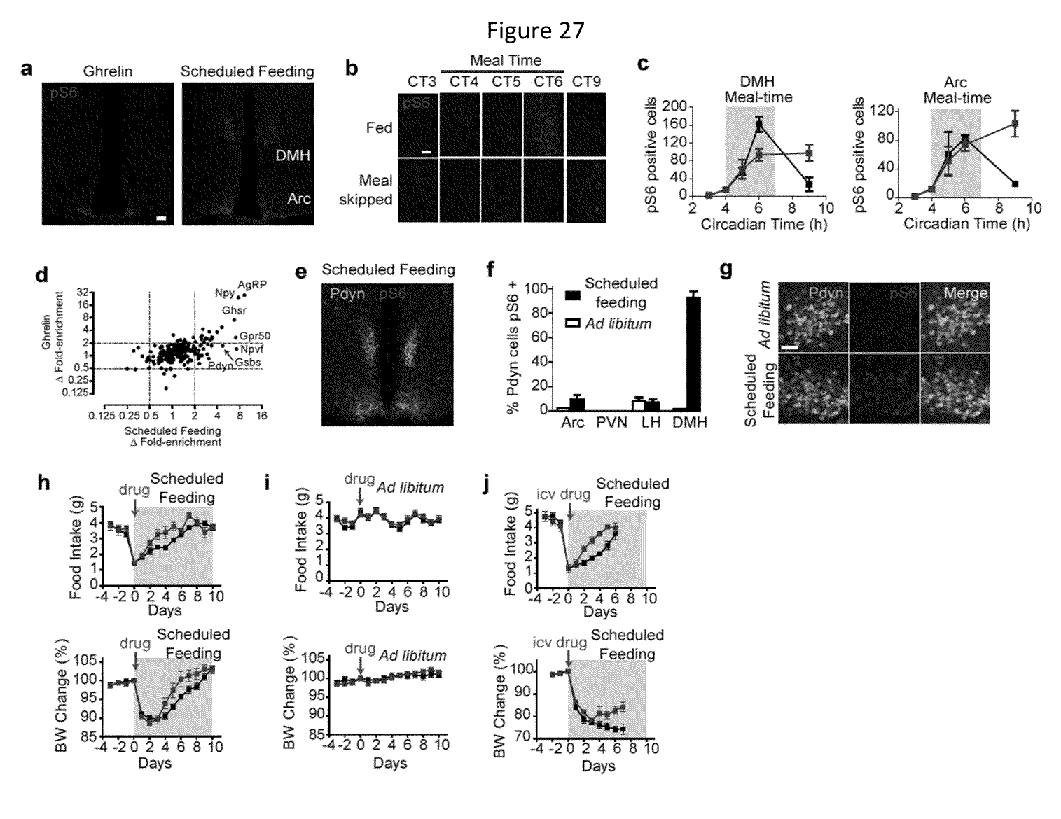

The access of mice to food was restricted to a three-hour window in the middle of the light phase (circadian time 4-7), which resulted in the emergence of robust FAA within 7-10 days. pS6 staining of brain slices from these mice were performed at several time points to establish the dynamics of ribosome phosphorylation in the hypothalamus. It was found that scheduled feeding induced intense pS6 staining in the DMH and Arc (FIG. 27a) that peaked within the meal window and declined to baseline thereafter (FIG. 27b,c). This DMH staining was localized to the compact part of the DMH, a region which does not show a change in ribosome phosphorylation after a single overnight fast (FIG. 24a). Once the mice were entrained, this pattern of S6 phosphorylation no longer depended on the presence of food, since brain sections from mice that were acclimated to scheduled feeding but not fed on the day of the experiment showed a similar pattern of pS6 (although with lower intensity in the DMH; FIG. 27b). This suggests the existence of unidentified neural populations that are regulated in part by a circadian signal entrained by food availability.

To identify neurons activated during scheduled feeding, phosphorylated ribosomes were immunoprecipitated from the hypothalamus of animals sacrificed at the midpoint of the feeding window and analyzed the enriched mRNAs. To provide a comparison data set, ribosome profiling was also performed from mice that received an injection of the hormone ghrelin. Levels of plasma ghrelin can increase prior to meals and this increase has been hypothesized to promote scheduled feeding (Mistlberger, R. E. Physiology & behavior 104, 535-545, doi:10.1016/j.physbeh.2011.04.015 (2011); Verhagen, L. A. et al. European neuropsychopharmacology 21, 384-392, doi:10.1016/j.euroneuro.2010.06.005 (2011); LeSauter, J., et al. Proc Natl Acad Sci USA 106, 13582-13587, doi:10.1073/pnas.0906426106 (2009); each of which is hereby incoporated by reference in its entirety). It was found that ghrelin induced strong pS6 in the Arc but did not increase pS6 levels in the compact part of the DMH (FIG. 27a). Thus these two profiles were compared in order to segregate enriched cell-type markers according to their potential anatomic location (e.g., DMH versus Arc) and function.

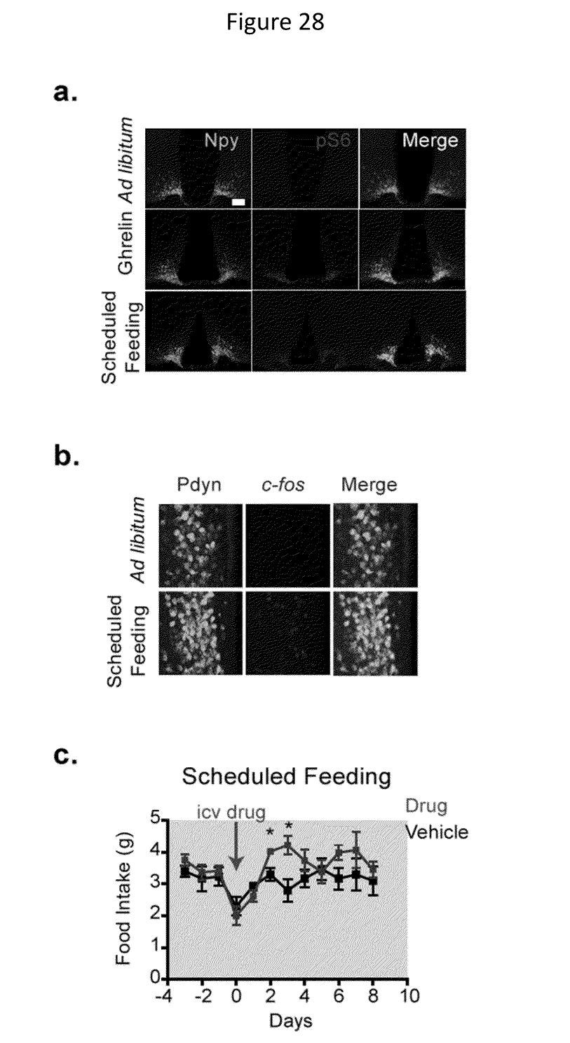

Ghrelin and scheduled feeding both induced strong enrichment of AgRP (27 and 9.2-fold), NPY (21 and 7.8-fold), and the ghrelin receptor (7.1 and 6.8-fold) and extensive co-localization between pS6 and AgRP/NPY neurons of the Arc was confirmed in both settings (FIG. 28a). The activation of AgRP/NPY neurons is consistent with the voracious eating displayed by animals acclimated to scheduled feeding following food presentation, and suggests that ghrelin and scheduled feeding activate a common set of neural targets in the Arc.

### Molecular Anatomy and Function of the DMH

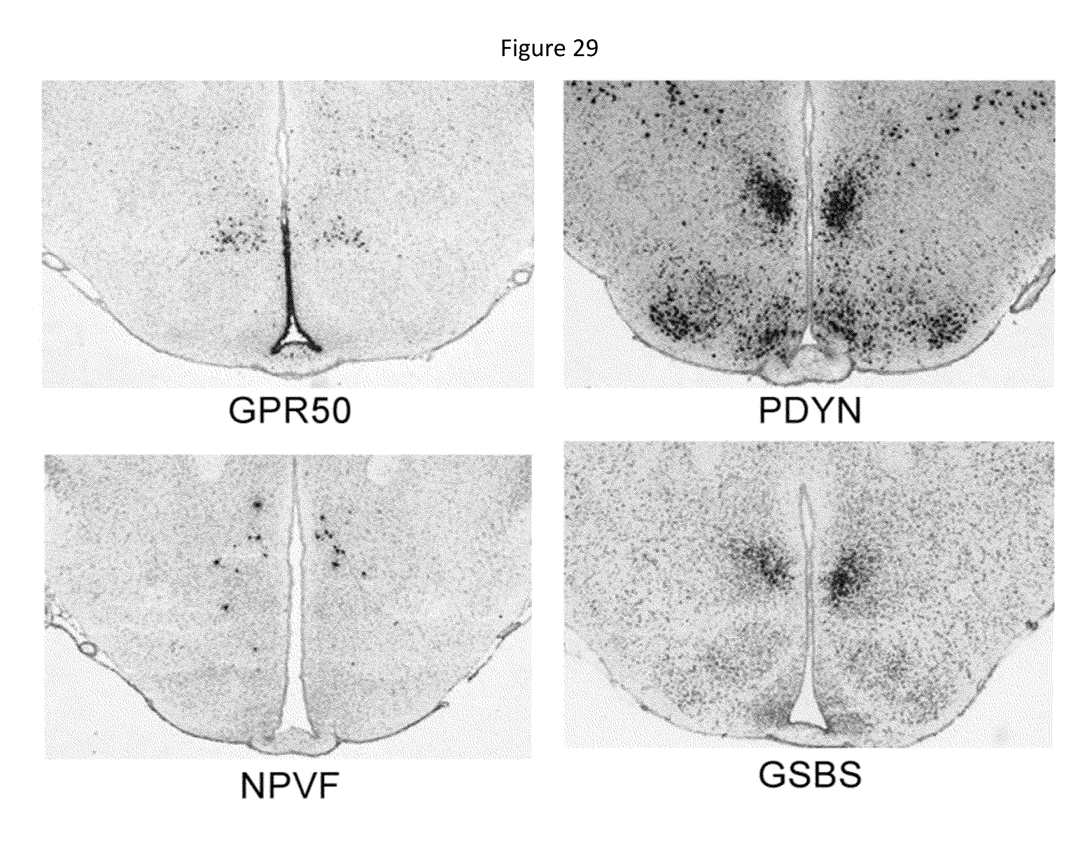

The next experiments focused on identifying the activated neurons in the DMH, since the understanding of the function and identity of the cell-types in this region that regulate feeding is limited. Four transcripts—Npvf, Pdyn, Gpr50, and Gsbs—were identified that were enriched in pS6 immunoprecipitates from mice subjected to scheduled feeding relative to ghrelin (FIG. 27d) and it was confirmed that these transcripts showed localized expression in the DMH based on analysis of in situ hybridization data from the Allen Brain Atlas (FIG. 29). Among these, neuropeptide Npvf (also known as RF amide) has previously been shown to co-localize with c-fos in a sparse population of DMH cells activated during FAA (Acosta-Galvan, G. et al. Proc Natl Acad Sci USA 108, 5813-5818, doi:10.1073/pnas.1015551108 (2011); hereby incorporated by reference in its entirety) and the G-protein coupled receptor Gpr50 can be regulated by leptin and nutritional state (Ivanova, E. A. et al. Am J Physiol Endocrinol Metab 294, E176-182, doi:10.1152/ajpendo.00199.2007 (2008); hereby incorporated by reference in its entirety) but has not previously been linked to scheduled feeding.

The next experiments characterized in greater detail the neurons in the DMH that express Pdyn, a neuropeptide that can have complex effects on mood, nociception, and reward but that has not previously been linked to scheduled feeding. Immunostaining revealed extensive co-localization between Pdyn and pS6 across the entire rostrocaudal axis of the DMH in brain sections from mice sacrificed two hours after food presentation (FIG. 27e): 92% of Pdyn neurons in the DMH showed pS6 staining in mice subjected to scheduled feeding compared to just 2% in ad libitum controls (FIG. 27f,g). Little co-localization was observed between pS6 and Pdyn in other hypothalamic regions such as the PVN, Arc, or lateral hypothalamus (FIG. 27f), suggesting that the Pdyn neurons in the DMH represent a functionally distinct population with a specialized role in feeding. c-fos staining revealed extensive co-localization between c-fos and Pdyn in the DMH during scheduled but not ad libitum feeding (FIG. 28b), confirming that Pdyn neurons are selectively activated when mice are exposed to this protocol. Thus Pdyn neurons in the DMH represent a novel population of neurons with a potential functional link to scheduled feeding.

It was hypothesized that Pdyn might play a role in meal termination following bouts of intense feeding. This hypothesis was based on the observation that pS6 induction in Pdyn neurons is evident only late in the meal window (FIG. 27b,c), requires food presentation for full expression (FIG. 27b,c), and is not observed in response to orexigenic signals such as fasting or ghrelin (FIG. 24a and FIG. 27a). Pdyn can signal by activating the K-opioid receptor (KOR), and potent, highly selective KOR antagonists have been described (Gai, W. P., et al. The Journal of comparative neurology 298, 265-280, doi:10.1002/cne.902980302 (1990) and Sherman, T. G., et al. Neuroendocrinology 44, 222-228 (1986); each of which is incorporated by reference in its entirety). The function of Pdyn during scheduled feeding was therefore assessed using pharmacological inhibitors of KOR. An intraperitoneal injection of either a selective KOR antagonist (JDTic) or vehicle was administered to mice, and then assigned animals to two groups: one exposed to the scheduled feeding paradigm and the other fed ad libitum. Because KOR antagonists can have a characteristic long duration of action in vivo (up three weeks), only a single dose was used (Bruchas, M. R. et al. J Biol Chem 282, 29803-29811, doi:10.1074/jbc.M705540200 (2007) and Koch, C., Crick, F. The Neuronal Basis of Consciousness (1999); each of which is incoporated by reference in its entirety).

Vehicle treated animals initially consumed less food per day and lost weight after shifting to scheduled feeding, after which their weight gradually recovered over the course of seven days (FIG. 27h; black). Mice that were treated with JDTic showed a similar decrease in food intake and body weight at first, but relative to the control group, their food intake increased more rapidly, and they showed a more rapid regain of body weight (FIG. 27h; gray). Remarkably, JDTic had no impact on food intake or body weight in ad libitum fed animals (FIG. 27i), indicating that the increased feeding induced by the drug is only evident under conditions where the Pdyn neurons are activated. JDTic was next delivered by intracerebroventricular (icy) injection and observed a similar increase in food intake for mice on a scheduled feeding paradigm, indicating that these effects are mediated by central KOR signaling (FIG. 28c). This was confirmed by testing a second, structurally unrelated KOR antagonist (norbinaltorphimine), which induced a dramatic (more than 50%) increase in food intake when delivered icy to animals on a scheduled feeding protocol (FIG. 27j). Taken together, these data indicate that Pdyn neurons in the DMH are selectively activated during scheduled feeding, and that the resultant Pdyn signaling acts to limit food intake following intense feeding. This illustrates how ribosome profiling can enable the identification and functional analysis of molecularly defined populations of neurons.

Phosphorylation of Ribosomal Protein S6 can be Used as a Tag to Enable the Capture of mRNA from Activated Cells

A vast array of experiments have sought to establish the functional importance of discrete neurons in controlling behavior (Lichtman, J. W. & Denk, W. Science 334, 618-623, doi:10.1126/science.1209168 (2011); hereby incorporated by reference in its entirety). However, these efforts can be limited by a lack of molecular information about the relevant cell-types. In 1999 Francis Crick and Christof Koch predicted that the development of techniques “based on the molecular identification and manipulation of discrete and identifiable subpopulations” of neurons (Koch, C., Crick, F. The Neuronal Basis of Consciousness (1999); hereby incorporated by reference in its entirety) would enable the elucidation of the functional anatomy of the CNS. With the development of optogenetics and related methods, the means for manipulating cells are now available. By contrast there has been less progress toward the development of approaches for the molecular identification of functional populations of neurons, and for many neural functions the molecular identity of the relevant cell-types remains unknown (Zhang, F., et al. Nature reviews. Neuroscience 8, 577-581, doi:10.1038/nrn2192 (2007); Lin, D. et al. Nature 470, 221-226, doi:10.1038/nature09736 (2011); and Wu, Q., et al. Nature 483, 594-597, doi:10.1038/nature10899 (2012); each of which is incorporated by reference in its entirety). This problem of linking cell-types to function has persisted despite increasingly sophisticated measurements of the molecular heterogeneity of the brain as a whole (Gong, S. et al. Nature 425, 917-925, doi:10.1038/nature02033 (2003) and Lein, E. S. et al. Nature 445, 168-176, doi:10.1038/nature05453 (2007); hereby incorporated by reference in its entirety).

The results presented herein demonstrate a conceptually new way to map the functional organization of gene expression in the brain. This approach takes advantage of the fact that marker genes can be used to identify specific cell-types within an anatomic region such as the hypothalamus (Siegert, S. et al. Nature neuroscience 12, 1197-1204, doi:10.1038/nn.2370 (2009); hereby incorporated by reference in its entirety). The result demonstrate that it is possible to capture RNA from cells in proportion to their activity, quantify the enrichment of these cell-type specific marker genes, and then use this information to assay in parallel changes in the functional state of a large number of intermingled cell-types. An advantage of this approach is that is enables the use of powerful tools of molecular biology, such as qPCR or RNA sequencing, to make a measurement of cellular activity that would otherwise require analysis of large numbers of samples by histology. As a result it is possible to identify in an unbiased way the specific genes that are most uniquely expressed in a co-regulated population of neurons in the brain. Once identified, such genes can serve as markers that enable the functional interrogation of those cells using optogenetics or other approaches.

In this example, it was demonstrated that phosphorylation of ribosomal protein S6 can be used as a tag to enable the capture of mRNA from activated cells. This is possible because the signaling pathways that trigger S6 phosphorylation are often themselves correlated with neural activity (Valjent, E. et al. Neuropsychopharmacology 36, 2561-2570, doi:10.1038/npp.2011.144 (2011); Meyuhas, O. International review of cell and molecular biology 268, 1-37, doi:10.1016/51937-6448(08)00801-0 (2008); and Flavell, S. W. & Greenberg, M. E. Annual review of neuroscience 31, 563-590, doi:10.1146/annurev.neuro.31.060407.125631 (2008); each of which is incorporated by reference in its entirety). As the phosphorylation sites on S6 are evolutionarily conserved (Meyuhas, O. International review of cell and molecular biology 268, 1-37, doi:10.1016/S1937-6448(08)00801-0 (2008); hereby incorporated by reference in its entirety), this approach can in principle can be used to study a wide array of vertebrate and invertebrate species, including those that are not amenable to genetic modification. Moreover, as S6 phosphorylation can be controlled by extracellular stimuli in all cells, this strategy could also reveal the regulation of non-neuronal cell-types that reside in other complex tissues besides the brain, such as the immune system, lung, intestine, kidney and others.

The fidelity of this approach was been validated by identifying many neurons known to be activated or inhibited in response to well-characterized stimuli such as salt challenge and fasting. In addition to recapitulating the known components of these systems, markers for activated neurons that have been overlooked have also been identified, such as Gal neurons during fasting and Pdyn neurons during scheduled feeding, or are expressed at low levels and therefore are challenging to detect by histology, such as the neuropeptide Rln1. As many functional populations of neurons have been visualized by c-fos staining but not molecularly characterized (Lin, D. et al. Nature 470, 221-226, doi:10.1038/nature09736 (2011); Dielenberg, R. A., et al. Neuroscience 104, 1085-1097 (2001); and Wu, Q., et al. Nature 483, 594-597, doi:10.1038/nature10899 (2012); each of which is hereby incorporated by reference in its entirety), phosphorylated ribosome profiling can provide a general way to identify these cells.

The finding that Pdyn neurons in the DMH are selectively activated during scheduled feeding reveals a new role for opioid peptides in the control of food intake. Whereas most research using this paradigm has focused on the signals that drive meal anticipation (Mistlberger, R. E. Physiology & behavior 104, 535-545, doi:10.1016/j.physbeh.2011.04.015 (2011); hereby incorporated by reference in its entirety), it was unexpectedly fount that Pdyn neurons act to limit food intake following bouts of intense feeding. Moreover this function appears to be specific to the scheduled feeding protocol in which Pdyn neuron activation was observed, as an effect of KOR antagonists was not detected on food intake in ad libitum fed animals (FIG. 27i) and indeed, in other contexts KOR antagonists have been reported to inhibit feeding (Jewett, D. C. et al. Brain research 909, 75-80 (2001); hereby incorporated by reference in its entirety). Understanding how dynorphin signaling is able to selectively regulate episodic feeding will require further characterization of the Pdyn cells in the DMH and their relation to other elements of the circuitry that controls food intake.

The disclosures of each and every patent, patent application, and publication cited herein are hereby incorporated herein by reference in their entirety.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of isolating actively translated mRNA from a first subpopulation of cells, said method comprising:
contacting a lysate or fraction of a heterogeneous population of cells with a reagent, said heterogeneous population of cells comprising said first subpopulation of cells and a second subpopulation of cells;
allowing said reagent to selectively bind to a protein comprising one or more posttranslational modifications, said protein being in a ribosome bound to said actively translated mRNA,
(i) wherein said first and said second subpopulation of cells comprise more than one of said protein,
(ii) wherein a greater percentage of said protein comprises at least one of said one or more posttranslational modifications in said first subpopulation of cells than in said second subpopulation of cells; and
isolating said actively translated mRNA from said lysate or fraction of the heterogeneous population of cells, thereby isolating actively translated mRNA from said first subpopulation of cells.

2. (canceled)

3. (canceled)

4. (canceled)

5. (canceled)

6. The method of claim 1, wherein said reagent binds to said protein at one or more sites of said one or more posttranslational modifications.

7. (canceled)

8. (canceled)

9. (canceled)

10. The method of claim 1, wherein said reagent is selected from the group consisting of a phospho-S6 240/244 antibody, a phospho-S6 235/236 antibody, a phospho-S6 244 antibody, and a fragment thereof.

11. The method of claim 1, wherein said reagent specifically binds to said protein at two or more sites.

12. The method of claim 11, wherein said two or more sites comprises at least one of said one or more posttranslational modifications.

13. The method of claim 11 further comprising a peptide that is at least about 60%, 70%, 80%, 90%, 95%, 99%, or 100% identity to SEQ ID NO:25.

14. (canceled)

15. (canceled)

16. (canceled)

17. (canceled)

18. The method of claim 1, wherein said protein is ribosomal protein S6 and said one or more posttranslational modifications comprise phosphorylation at serine 235, serine 236, serine 240, serine 244, serine 247, or a combination thereof.

19. (canceled)

20. (canceled)

21. The method of claim 1, wherein at least one of said one or more posttranslational modifications occurs in response to a stimulus.

22. (canceled)

23. (canceled)

24. (canceled)

25. (canceled)

26. (canceled)

27. A method for identifying mRNA whose translation is modulated in response to a stimulus, said method comprising:
contacting a lysate or fraction of a heterogeneous population of cells with a reagent,
(i) wherein said stimulus has been applied to a source of said heterogeneous population of cells,
(ii) wherein said heterogeneous population of cells comprises a protein comprising one or more posttranslational modifications, and
(iii) wherein at least one of said one or more posttranslational modifications occurs in response to said stimulus;
allowing said reagent to selectively bind to said protein comprising said one or more posttranslational modifications, said protein being in a ribosome bound to said mRNA;
isolating said ribosome bound to said reagent and said mRNA;
determining an identity and an amount of said mRNA in said isolated ribosome;
determining an identity and an amount of said mRNA in a control sample; and
comparing, for mRNA of common identity, said amount of said mRNA in the isolated ribosome to said amount of said mRNA in said control sample, thereby identifying mRNA whose translation is modulated in response to said stimulus.

28. A method for identifying cell types that are activated in response to a stimulus, said method comprising:
contacting a lysate or fraction of a heterogeneous population of cells with a reagent,
(i) wherein said stimulus has been applied to a source of said heterogeneous population of cells,
(ii) wherein said heterogeneous population of cells comprises a protein comprising one or more posttranslational modifications, and
(iii) wherein at least one of said one or more posttranslational modifications occurs in response to said stimulus;
allowing said reagent to selectively bind to said protein comprising said one or more posttranslational modifications, said protein being in a ribosome bound to said mRNA;
isolating said ribosome bound to said reagent and said mRNA;
determining an identity and an amount of said mRNA in said isolated ribosome;
determining an identity and an amount of said mRNA in a control sample;
comparing, for mRNA of common identity, said amount of said mRNA in said isolated ribosome to said amount of said mRNA in said control sample to identify a profile of two or more mRNA whose translation is modulated in response to said stimulus; and
correlating said profile of two or more mRNA to genetic markers associated with one or more cell types in said heterogeneous population of cells, thereby identifying cell types that are activated in response to said stimulus.

29. (canceled)

30. (canceled)

31. (canceled)

32. (canceled)

33. (canceled)

34. (canceled)

35. (canceled)

36. (canceled)

37. (canceled)

38. A method of isolating actively translated mRNA from activated cells, said method comprising:
contacting a lysate or fraction of a heterogeneous population of cells with a reagent, said heterogeneous population of cells comprising activated cells and unactivated cells;
allowing said reagent to selectively bind to phosphorylated ribosomal protein S6, said phosphorylated ribosomal protein S6 being in a ribosome bound to said actively translated mRNA,
(i) wherein said activated cells and unactivated cells comprise more than one of said ribosomal protein S6, and wherein a greater percentage of said ribosomal protein S6 is phosphorylated in said activated cells than in said unactivated cells; and
isolating said actively translated mRNA from said lysate or fraction of the heterogeneous population of cells, thereby isolating actively translated mRNA from activated cells.

39. The method of claim 38, wherein said isolating step comprises isolating said ribosome bound to said reagent and said actively translated mRNA.

40. The method of claim 38, further comprising identifying said actively translated mRNA.

41. The method of claim 38, further comprising determining an amount of said actively translated mRNA.

42. The method of claim 38, wherein said amount of said actively translated mRNA is normalized based on the amount of said mRNA in said lysate or fraction prior to contacting said lysate or fraction with said reagent.

43. A system for isolating actively translated mRNA from a first subpopulation of cells, said system comprising:
a lysate or fraction of a heterogeneous population of cells wherein said heterogeneous population of cells comprises a first subpopulation of cells and a second subpopulation of cells;
a reagent that selectively binds to a protein comprising one or more posttranslational modifications, said protein being in a ribosome bound to said actively translated mRNA,
(i) wherein said first and said second subpopulation of cells comprise more than one of said protein, and
(ii) wherein a greater percentage of said protein comprises at least one of said one or more posttranslational modifications in said first subpopulation of cells than in said second subpopulation of cells; and
a container configured to house said lysate or fraction and said reagent.

44. A kit for isolating actively translated mRNA from activated cells in a heterogeneous population of cells, said kit comprising:
an antibody or fragment thereof that binds to a single epitope of a phosphorylated S6 protein,
instructions for use.

45. A kit for isolating actively translated mRNA from activated cells in a heterogeneous population of cells, said kit comprising:
an antibody or fragment thereof that binds to a phosphorylated S6 protein at two or more epitopes;
a peptide that decreases the binding affinity of said antibody or fragment thereof to one or more epitopes on said phosphorylated S6 protein;
instructions for use.

46. An antibody or fragment thereof that binds to a single epitope of a phosphorylated S6 protein.

47. (canceled)

48. (canceled)

49. A hybridoma that expresses a monoclonal antibody or fragment thereof that binds to a single epitope of a phosphorylated S6 protein, wherein the single epitope comprises phosphorylated serine 244.

50. (canceled)

51. A peptide that decreases the binding affinity between one or more epitopes of a phosphorylated S6 protein and an antibody or fragment thereof that binds to two or more epitopes of the phosphorylated S6 protein.

52. The peptide of claim 51, wherein said peptide is phosphorylated.

53. The peptide of claim 51, wherein said peptide has at least about 60%, 70%, 80%, 90%, 95%, 99%, or 100% identity to SEQ ID NO:25.
